SEQUENCE LISTING (12 sequences)

SEQ ID NO: 1 - protein, 183 aa, Hepatitis B virus
MDIDPYKEFGATVELLSFLPSDFFPSVRDLLDTASALYREALESPEHCSPHHTALRQAILCWGELMTLATWVGGNLEDPISRDLVVSYVNTNMGLKFRQLLWFHISCLTFGRETVIEYLVSFGVWIRTPPAYRPPNAPILSTLPETTVVRRRGRSPRRRTPSPRRRRSQSPRRRRSQSRESQC

SEQ ID NO: 2 - protein, 148 aa, artificial sequence
  C-truncated Core (1-148aa)
MDIDPYKEFGATVELLSFLPSDFFPSVRDLLDTASALYREALESPEHCSPHHTALRQAILCWGELMTLATWVGGNLEDPISRDLVVSYVNTNMGLKFRQLLWFHISCLTFGRETVIEYLVSFGVWIRTPPAYRPPNAPILSTLPETTV

SEQ ID NO: 3 - protein, 832 aa, Hepatitis B virus
MPLSYQHFRRLLLLDDEAGPLEEELPRLADEGLNRRVAEDLNLGNLNVSIPWTHKVGNFTGLYSSTVPVFNPHWKTPSFPNIHLHQDIIKKCEQFVGPLTVNEKRRLQLIMPARFYPNVTKYLPLDKGIKPYYPEHLVNHYFQTRHYLHTLWKAGILYKRETTHSASFCGSPYSWEQKLQHGAESFHQQSSGILSRPPVGSSLQSKHRKSRLGLQSQQGHLARRQQGRSWSIRAGIHPTARRSFGVEPSGSGHSTNLASKSASCLYQSPVRKAAYPAVSTFEKHSSSGHAVELHNLPPNSARSQSERPVFPCWWLQFRNSKPCSDYCLSHIVNLLEDWGPCAEHGEHHIRIPRTPARVTGGVFLVDKNPHNTAESRLVVDFSQFSRGNYRVSWPKFAVPNLQSLTNLLSSNLSWLSLDVSAAFYHLPLHPAAMPHLLVGSSGLSRYVARLSSNSRIFNYQHGTMQNLHDSCSRNLYVSLLLLYQTFGRKLHLYSHPIILGFRKIPMGVGLSPFLLAQFTSAICSVVRRAFPHCLAFSYMDDVVLGAKSVQHLESLFTAVTNFLLSLGIHLNPNKTKRWGYSLHFMGYVIGCYGSLPQDHIIQKIKECFRKLPVNRPIDWKVCQRIVGLLGFAAPFTQCGYPALMPLYACIQSKQAFTFSPTYKAFLCKQYLNLYPVARQRPGLCQVFADATPTGWGLVMGHQRMRGTFLAPLPIHTAELLAACFARSRSGANILGTDNSVVLSRKYTSFPWLLGCAANWILRGTSFVYVPSALNPTDDPSRGRLGLSRPLLRLPFRPTTGRTSLYADSPSVPSHLPDRVHFASPLHVAWRPP

SEQ ID NO: 4 - protein, 832 aa, artificial sequence
  mutated polymerase (YMHD + RnaseH mutation)
MPLSYQHFRRLLLLDDEAGPLEEELPRLADEGLNRRVAEDLNLGNLNVSIPWTHKVGNFTGLYSSTVPVFNPHWKTPSFPNIHLHQDIIKKCEQFVGPLTVNEKRRLQLIMPARFYPNVTKYLPLDKGIKPYYPEHLVNHYFQTRHYLHTLWKAGILYKRETTHSASFCGSPYSWEQKLQHGAESFHQQSSGILSRPPVGSSLQSKHRKSRLGLQSQQGHLARRQQGRSWSIRAGIHPTARRSFGVEPSGSGHSTNLASKSASCLYQSPVRKAAYPAVSTFEKHSSSGHAVELHNLPPNSARSQSERPVFPCWWLQFRNSKPCSDYCLSHIVNLLEDWGPCAEHGEHHIRIPRTPARVTGGVFLVDKNPHNTAESRLVVDFSQFSRGNYRVSWPKFAVPNLQSLTNLLSSNLSWLSLDVSAAFYHLPLHPAAMPHLLVGSSGLSRYVARLSSNSRIFNYQHGTMQNLHDSCSRNLYVSLLLLYQTFGRKLHLYSHPIILGFRKIPMGVGLSPFLLAQFTSAICSVVRRAFPHCLAFSYMHDVVLGAKSVQHLESLFTAVTNFLLSLGIHLNPNKTKRWGYSLHFMGYVIGCYGSLPQDHIIQKIKECFRKLPVNRPIDWKVCQRIVGLLGFAAPFTQCGYPALMPLYACIQSKQAFTFSPTYKAFLCKQYLNLYPVARQRPGLCQVFADATPTGWGLVMGHQRMRGTFLAPLPIHTAHLLAACFARSRSGANILGTDNSVVLSRKYTSFPWLLGCAANWILRGTSFVYVPSALNPTDDPSRGRLGLSRPLLRLPFRPTTGRTSLYADSPSVPSHLPDRVHFASPLHVAWRPP

SEQ ID NO: 5 - protein, 792 aa, artificial sequence
  designed pol for TG1050 (del YMDDVVL + 4 RNase H mutations)
MPLSYQHFRRLLLLDDEAGPLEEELPRLADEGLNRRVAEDLNLGNLNVSIPWTHKVGNFTGLYSSTVPVFNPHWKTPSFPNIHLHQDIIKKCEQFVGPLTVNEKRRLQLIMPARFYPNVTKYLPLDKGIKPYYPEHLVNHYFQTRHYLHTLWKAGILYKRETTHSASFCGSPYSWEQKLQHGAESFHQQSSGILSRPPVGSSLQSKHRKSRLGLQSQQGHLARRQQGRSWSIRAGIHPTARRSFGVEPSGSGHSTNLASKSASCLYQSPVRKAAYPAVSTFEKHSSSGHAVELHNLPPNSARSQSERPVFPCWWLQFRNSKPCSDYCLSHIVNLLEDWGPCAEHGEHHIRIPRTPARVTGGVFLVDKNPHNTAESRLVVDFSQFSRGNYRVSWPKFAVPNLQSLTNLLSSNLSWLSLDVSAAFYHLPLHPAAMPHLLVGSSGLSRYVARLSSNSRIFNYQHGTMQNLHDSCSRNLYVSLLLLYQTFGRKLHLYSHPIILGFRKIPMGVGLSPFLLAQFTSAICSVVRRAFPHCLAFSGAKSVQHLESLFTAVTNFLLSLGIHLNPNKTKRWGYSLHFMGYVIGCYGSLPQDHIIQKIKECFRKLPVNRPIDWKVCQRIVGLLGFAAPFTQCGYPALMPLYACIQSKQAFTFSPTYKAFLCKQYLNLYPVARQRPGLCQVFAHATPTGWGLVMGHQRMRGTFLSRKYTSFPWLLGCAANWILRGTSFVYYPSALNPYHDPSRGRLGLSRPLLRLPFRPTTGRTSLYADSPSVPSHLPDRVHFASPLHVAWRPP

SEQ ID NO: 6 - protein, 39 aa, artificial sequence
  env1 domain
MVLQAGFFLLTRILTIPQSLDSWWTSLNFLGGTTVCLGQ

SEQ ID NO: 7 - protein, 30 aa, artificial sequence
  env2 domain
WASARFSWLSLLVPFVQWFVGLSPTVWLSV

SEQ ID NO: 8 - protein, 1007 aa, artificial sequence
  Fusion TG1050 core(1-148)-pol del YMDDVVL + del RNAse H domain) + env1 + env2
MDIDPYKEFGATVELLSFLPSDFFPSVRDLLDTASALYREALESPEHCSPHHTALRQAILCWGELMTLATWVGGNLEDPISRDLVVSYVNTNMGLKFRQLLWFHISCLTFGRETVIEYLVSFGVWIRTPPAYRPPNAPILSTLPETTVPLSYQHFRRLLLLDDEAGPLEEELPRLADEGLNRRVAEDLNLGNLNVSIPWTHKVGNFTGLYSSTVPVFNPHWKTPSFPNIHLHQDIIKKCEQFVGPLTVNEKRRLQLIMPARFYPNVTKYLPLDKGIKPYYPEHLVNHYFQTRHYLHTLWKAGILYKRETTHSASFCGSPYSWEQKLQHGAESFHQQSSGILSRPPVGSSLQSKHRKSRLGLQSQQGHLARRQQGRSWSIRAGIHPTARRSFGVEPSGSGHSTNLASKSASCLYQSPVRKAAYPAVSTFEKHSSSGHAVELHNLPPNSARSQSERPVFPCWWLQFRNSKPCSDYCLSHIVNLLEDWGPCAEHGEHHIRIPRTPARVTGGVFLVDKNPHNTAESRLVVDFSQFSRGNYRVSWPKFAVPNLQSLTNLLSSNLSWLSLDVSAAFYHLPLHPAAMPHLLVGSSGLSRYVARLSSNSRIFNYQHGTMQNLHDSCSRNLYVSLLLLYQTFGRKLHLYSHPIILGFRKIPMGVGLSPFLLAQFTSAICSVVRRAFPHCLAFSVLQAGFFLLTRILTIPQSLDSWWTSLNFLGGTTVCLGQGAKSVQHLESLFTAVTNFLLSLGIHLNPNKTKRWGYSLHFMGYVIGCYGSLPQDHIIQKIKECFRKLPVNRPIDWKVCQRIVGLLGFAAPFTQCGYPALMPLYACIQSKQAFTFSPTYKAFLCKQYLNLYPVARQRPGLCQVFAHATPTGWGLVMGHQRMRGTFLWASARFSWLSLLVPFVQWFVGLSPTVWLSVSRKYTSFPWLLGCAANWILRGTSFVYYPSALNPYHDPSRGRLGLSRPLLRLPFRPTTGRTSLYADSPSVPSHLPDRVHFASPLHVAWRPP

SEQ ID NO: 9 - DNA, 3024 bp, artificial sequence
  nt sequence eencoding fusion TG1050 (core 1-148 pol delete pol et RNAse H domains-env1-env2)
atggatatcgatccctacaaggagttcggtgccaccgtcgaactgctgagcttcctgcccagcgatttcttcccaagcgtacgtgaccttctagatacagcctcagctctgtatcgggaagccttagagtctcctgagcattgttcacctcaccatactgctctcaggcaagcaattctgtgctggggagaactaatgactctagctacctgggtgggtggtaatttggaagatccaatatccagggacctagtagtcagttatgtcaacactaatatgggactaaagttccgacaactattgtggtttcacatttcttgtctcacttttggaagagaaacagttatagaatatttggtgtctttcggagtgtggattcgcactcctccagcttatagaccaccaaacgcaccgatactgagcaccctgccagaaaccaccgtgccactgagctaccagcactttcgcaggctgttactgctggatgatgaagctggaccgctggaggaggagctgccacgtctggctgatgagggactgaaccgtcgtgtggctgaggacctgaacctgggcaacctgaacgtgagcattccttggactcataaggtgggaaactttacgggactttattcttctactgtacctgtctttaaccctcattggaaaacaccctcttttcctaatatacatttacaccaagacattatcaagaaatgtgaacaatttgtaggaccactcacagtcaatgagaaaagaagactgcaattgattatgcctgctaggttttatccaaatgttaccaaatatttgccattggataagggtattaaaccttattatccagaacatctagttaatcattacttccaaaccagacattatttacacactctatggaaggcaggtatattatataagagagaaacaacacatagtgcctcattttgtgggtcaccatattcttgggaacaaaagctacagcatggagcagaatctttccaccagcaatcctctgggattctttcccgaccaccagttggatccagccttcagagcaaacaccgcaaatccagattgggacttcaatcccaacaaggacacctggccagacgccaacaaggtaggagctggagcattcgagctgggattcaccccaccgcacggaggtcttttggggtggagccctcaggctcagggcattctacaaaccttgccagcaaatcagcctcctgcctctaccaatcgccagtcaggaaggcagcctaccctgctgtctccacctttgagaaacactcatcctcaggccatgcagtggaactccacaaccttccaccaaactctgcaagatcccagagtgagaggcctgtatttccctgctggtggctccagttcaggaacagtaaaccctgttccgactactgtctctcccatatcgtcaatcttctcgaggattggggaccctgcgctgaacacggtgagcaccatattcgcatcccgagaacgccagcacgcgtgaccggtggcgtgttcctggtggataagaacccacataacacggctgaaagccgtctggttgttgactttagccagttcagccgtggcaattatcgcgttagctggcctaagtttgcggtgccgaatctgcagagcctgacgaatctgttaagcagcaatttaagctggctgagcttagacgttagcgcagccttctaccacctgccactgcacccagcagcgatgccacacctgctggtgggcagcagcggtctgagccgttacgtggcacgcctgagcagcaacagccgtatatttaattatcaacatggcacgatgcaaaatttacatgatagctgtagccgtaatctgtacgtgagcctgttactgttatatcagacgtttggtcgcaagctgcatttatacagccacccgattattttagggttccgcaagatcccgatgggtgttggtctgtctccattcctgttagcgcaattcaccagcgcaatctgcagcgttgtgcgcagagcgtttccgcattgcctggcgtttagcgtcctgcaagcaggcttcttcctgctgacccgtattctgaccattccacaaagcctggatagctggtggaccagcctgaacttcctgggtggcaccacggtttgcctgggtcagggtgcgaaaagcgtgcaacacctggaaagcctgttcacggcagtgacgaacttcctgctgagcctgggcattcacctgaaccctaacaaaacaaagagatggggttactctttacatttcatgggctatgtcattggatgttatgggtcattgccacaagatcacatcatacaaaagatcaaagaatgttttcgaaaacttcctgttaacagacctattgattggaaagtctgtcaacgtattgtgggtcttttgggttttgctgctccttttacacaatgtggttatcctgctttaatgcctttgtatgcatgtattcagtcgaagcaggcttttactttctcgccaacttacaaggcctttctgtgtaaacaatacctgaacctttaccctgttgctcggcaaagaccaggtctgtgccaagtgtttgctcacgcaacccctactggctggggattggtcatgggacatcagcgcatgcgtggaacctttctgtgggcaagcgcacgctttagctggctgagcctgctggttccgttcgtgcaatggtttgtgggtctgagcccaaccgtgtggctgagcgtgtccagaaaatatacatcgtttccatggctgctaggctgtgctgccaactggatactgagaggcaccagcttcgtgtattatcccagcgctctcaacccttaccatgatccttctcgaggtagactgggcctgagcagacctctgctgagactccccttccgacccacaaccggaagaacaagcctgtatgccgatagccctagcgtccccagccacctccctgatagagtccattttgccagcccactccatgtggcctggaggcctccctaa

SEQ ID NO: 10 - protein, 10 aa, artificial sequence
  VSA Pol-specific peptide
VSAAFYHLPL

SEQ ID NO: 11 - protein, 15 aa, artificial sequence
  Pol-specific N13F pepide
NLNVSIPWTHKVGNF

SEQ ID NO: 12 - protein, 9 aa, artificial sequence
  Ad-specific FAL peptide
FALSNAEDL

(19) United States
(12) Patent Application Publication
(10) Pub. No.: US 2019/0328869 A1
(43) Pub. Date: Oct. 31, 2019

(54) IMMUNOTHERAPEUTIC PRODUCT AND MDSC MODULATOR COMBINATION THERAPY

(71) Applicant: Transgene SA, Illkirch Graffenstaden (FR)

(72) Inventors: Karine Lelu-Santolaria, Pont-Eveque (FR); Roland Kratzer, Lyon (FR); Perrine Martin, L'isle D'abeau (FR)

(73) Assignee: Transgene SA, Illkirch Graffenstaden (FR)

(21) Appl. No.: 16/339,417

(22) PCT Filed: Oct. 10, 2017
(86) PCT No.: PCT/EP2017/075808

§ 371 Date: Apr. 4, 2019

(30) Foreign Application Priority Data
Oct. 10, 2016 (EP) 16306333.2
Jan. 17, 2017 (EP) 17305048.5

(51) Int. Cl. A61K 39/29; A61K 31/519; A61P 31/20; A61K 39/235

(52) U.S. Cl. CPC A61K 39/292; A61K 31/519; A61K 2039/5256; A61K 39/235; A61P 31/20

(57) ABSTRACT

The present invention provides an immunotherapeutic composition for use in combination with one or more MDSC (Myeloid-derived suppressor cells) modulator(s) and a kit of parts comprising such components as well as methods using such components in combination. The invention also provides the use of Phosphodiesterase-5 (PDE5) inhibitors for reversing immunosuppression in chronic infectious diseases. The invention is of very special interest in treating or preventing diseases, especially chronic infectious diseases such as hepatitis B.

### FIELD OF THE INVENTION

The present invention generally relates to the field of immunotherapy. Embodiments include an immunotherapeutic composition for use in combination with one or more MDSC (Myeloid-derived suppressor cells) modulator(s) and a kit of parts comprising such components as well as methods using such components in combination. The invention also provides the use of Phosphodiesterase-5 (PDE5) inhibitors for reversing immunosuppression in chronic infectious diseases. The invention is of very special interest for treating or preventing diseases, especially chronic infectious diseases such as hepatitis B.

### BACKGROUND OF THE INVENTION

Immunotherapy seeks to boost the host's immune system to help the body to eradicate pathogens and abnormal cells. Widely used in traditional vaccination, immunotherapy is also being actively investigated as a potential modality for treating severe, chronic or life-threatening diseases in an attempt to stimulate specific and innate immune responses. In particular, several viral and non-viral vectors have now emerged, all of them having relative advantages and limits making them more appropriate to certain indications (see for example Harrop and Carroll, 2006, Front Biosci., 11, 804-817; Inchauspé et al., 2009, Int Rev Immunol 28(1): 7-19; Torresi et al., 2011, J. Hepatol. 54(6): 1273-85). For example, viral vectors such as adenovirus (Ad) (Drummer et al., 2008, Mol Ther 16(5): 985-94; Dreno et al., 2014, PLoS One 9(2): e83670; Hemminki et al., 2015, Oncotarget 6(6): 4467-81) and vaccinia virus (Fournillier et al., 2007, Vaccine 25(42): 7339-53; Quoix et al., 2011, The Lancet Oncology 12(12): 1125-33; Boukhebza et al., 2012, Vaccines & Immunotherapeutics 8(12): 1746-57) among many others have now entered clinical development both in cancer and infectious diseases fields. Several encouraging strategies have focused on immunotherapeutic approaches that simultaneously target multiple HBV (Human hepatitis B Virus) antigens (e.g. Depla et al., 2008, J. Virol. 82: 435; WO2005/056051; WO2008/020656). For example, immunization of mice with an adenovirus vaccine encoding HBV polymerase, HBcAg and HBsAg domains was shown to elicit T cell responses against all expressed HBV antigens in preclinical mouse models (Martin et al., 2015, Gut. 64(12):1961-71).

However, there are limits on the immune system's ability to fight chronic diseases and cancers for several reasons. Importantly, diseased cells have evolved potent immunosuppressive mechanisms for eluding the immune system, posing a major obstacle to effective immunotherapy. Regulatory T (Treg) cell-mediated immune suppression at tumor site is now well documented (Lindau et al., 2013, Immunol. 138(2): 105-15; Facciabene et al., 2012, Cancer Res; 72(9): 2162-71). Hence, overcoming such immune blocking mechanisms may be key to successful development of more effective immunotherapeutics in cancer and infectious disease fields.

MDSCs (myeloid-derived suppressor cells) are typically defined as a heterogeneous population of cells of myeloid origin (development from a common myeloid progenitor), immature state and ability to potently suppress T cell responses. Under normal physiological conditions, they are involved in preventing uncontrolled immune reactions but strongly expand under pathological conditions such as cancer and chronic infections (e.g. in inflammatory conditions) where they play a critical role in T cell immunosuppression and induction of oxidative stress or amino acid starvation. MDSCs suppress T cell responses by various mechanisms including but not limited to production of reactive oxygen species, peroxynitrites, increased arginase metabolism. MDSCs also accelerate angiogenesis, tumor progression and metastasis through the expression of cytokines and factors such as TGF-beta.

Two main subsets were described in mice and humans as monocytic MDSCs (mMDSC) and granulocytic MDSCs (gMDSC) which show different and not exclusive phenotypic biomarker expression. In mice, MDSCs are characterized by expression of high levels of CD11b (a classical myeloid lineage marker) and GR1 (a granulocytic marker). GR1 is made up of two cell membrane molecules, Ly6C and Ly6G, and MDSCs are classified into monocytic and granulocytic, according to their relative expression levels. Monocytic MDSCs express high levels of the Ly6C surface marker with low or no expression of the Ly6G marker (CD11b⁺/Ly6C^{high}/LY6G⁻), while gMDSCs express Ly6C and high levels of Ly6G (CD11b⁺/Ly6C^{int}/LY6G⁺). Human MDSCs are less characterized. They are generally defined as myeloid cells expressing CD33 and CD11b with CD11b⁺/CD14⁺/CD15⁻/CD33⁺/HLA-DR^{-/low} signature for mMDSC and CD11b⁺/CD14⁻/CD15⁺/CD33⁺ for gMDSC (for a review, see e.g. Damuzzo et al., 2015, Cytometry Part B (Clinical Cytometry) 886:77-91).

Preclinical evidence in various mouse models has shown that Gr1(+) CD11b(+) MDSCs were enriched in melanoma lesions and lymphatic organs during tumor progression. MDSC infiltration was associated with a strong TCR ζ-chain down-regulation in all T cells (Meyer et al., 2011, Proc Natl Acad Sci 108(41): 17111-6). Importantly, the MDSC activation in a pathological context resulted in the upregulated expression of immune suppressive factors such as arginase and inducible nitric oxide (NO) synthase and increased production of NO and reactive oxygen species (Gabrilovich and Nagarej, 2009, Nat Rev Immunol 9(3): 162-74). In clinical settings, the number of circulating MDSCs is associated with clinical stages and metastatic tumor burden in several cancers. Moreover, MDSCs also play a role in gaining chemoresistant phenotype (Katoh and Watanabe, 2015, Mediators of Inflammation Article ID 159269).

MDSCs could also contribute to immune suppression in chronic viral infections. HBV-infected patients who resolved infection mounted multi-specific and sustained responses mediated by T helper (T_{H}) and cytotoxic T (CTL) lymphocytes and the appearance of neutralizing anti-HBe and anti-HBsAg-specific antibodies indicates a favorable outcome of infection. In contrast, the immune system is ineffective to clear viral infection in chronic hepatitis B patients. The reason for this alteration of the effector functions of the cellular immune response is currently not well-understood and considerable insight has been developed to understand the involvement of different inhibitory pathways responsible for HBV persistent infection and progression to HBV-related hepato-carcinoma. Kondo and Shimosegawa (2015, Int J Mol Sci 16: 3307-22) recently reported the contribution of immune suppressive MDSCs to the difficulty in inducing an effective immune response. A contrario, a decline in the frequency of circulating MDSCs was associated with an enhanced antibody response to HBV vaccine (Anthony et al., 2011, Vaccine 29: 3558-63). A higher percentage of MDSCs, defined as CD14⁺HLA-DR^{-/low}, has been detected in the peripheral blood of chronic hepatitis B patients compared with healthy control subjects. These cells have been shown to suppress HBV-specific CD8⁺ T cell responses (Huang et al., 2014, J Immunol 193: 5461-9). Pallett et al (2015, Nature Medicine 21(6): 591-600) provide evidence for the implication of granulocytic MDSCs in the immune regulation in HBV infected patients. These cells are expanded in patients in the immunotolerant (non-inflammatory) and inactive phases and might suppress T cells through arginase I-dependent mechanisms. Fang et al. (2015, J Immunol 195: 4873-83) recently reported that HBsAg could impair T cell activation by polarizing monocytes toward mMDSCs in an ERK/IL-6/STAT3 signaling-dependent manner. In addition, preclinical studies have provided evidence that MDSCs accumulate in liver of HBV-transgenic mouse models which significantly increased the capacity of suppressing proliferation of HBsAg-specific lymphocytes compared to normal mice-derived MDSCs (Chen et al., 2011, Clin Exp Immunol 166: 134-42).

Therefore, there is an increasing interest in the possible benefits of blocking MDSC immunosuppressive cells as a means of rescuing effective T cell immunity. Various approaches were disclosed during the last years to decrease MDSC amounts and inhibit MDSC-mediated immunosuppressive function under different pathological conditions (Gabrilovich and Nagarej, 2009, Nat Rev Immunol 9(3): 162-74; Ugel et al., 2009, Curr Opin Pharmacol 9(4): 470-81).

For example, antagonists may be used to block the tumor-induced factors that participate to MDSC's proliferation, expansion and mobilization into the inflammatory microenvironment (Pan et al., 2008, Blood 111(1): 219-28). Other strategies aim at decreasing circulating MDSCs with specific chemotherapy drugs combi (e.g. gemcitabine and 5-fluorouracile) (Annels et al., 2014, Cancer Immunol Immunother 63(2): 175-83) or impair the recruitment of MDSCs with monoclonal antibodies against molecules expressed on MDSC's surface such as GR1 and CXCR2 (Katoh et al., 2013, Cancer Cell 24(5): 631-44).

Another strategy would be promoting MDSC's differentiation into mature non-suppressive myeloid cells (dendritic cells, macrophage or granulocytes) using All-trans retinoic acid (ATRA), a vitamin A derivative (Nefedova et al., 2007 Cancer Res 67(22): 11021-8). In patients with metastatic renal cell carcinoma, ATRA administration decreased circulating CD33⁺HLA-DR⁻ MDSCs, which leads to improved myeloid/lymphoid DC ratio and antigen-specific T cell response (Mirza et al., 2006, Cancer Res 66(18): 9299-9307). In addition, 25-hydroxyvitamin D3 reduced the circulating CD34⁺ MDSCs in head and neck cancer patients although it failed to improve clinical outcome (Lathers et al., 2004, Cancer Immunol Immunother 53(5): 422-30). ATRA treatment was also shown to restore the proliferation and IFN-γ production by HBV-specific CD4⁺ and CD8⁺ T cells in PBMCs from chronically HBV infected patients (Fang et al., 2015, J Immunol 195: 4873-83). Treatments that reduce MDSC levels such as antibody depletion of Gr1 cells or treatments that down-regulate MDSC such as chemotherapy drugs or retinoic agents improve the efficacy of cancer vaccines or other immunotherapy in vivo (Chen et al., 2011, Clin Exp Immunol 166: 134-142).

Direct abrogation of MDSC's suppressive activities was also studied with compounds interacting with the pathways involved in MDSC-mediated immune suppression. Arginase-inhibitors like nor-NOHA have been used to abrogate MDSC function in vitro (Pallett et al., 2015, Nature Medicine 21(6): 591-600). Recently, phosphodiesterase (PDE)-5 inhibitors which are conventionally used for the treatment of erectile dysfunction, pulmonary hypertension and cardiac hypertrophy have been shown to reverse the suppressive machinery of tumor-recruited MDSCs in several mouse tumor models. Sildenafil (a PDE5 inhibitor) treatment was able to enhance intratumoral T cell infiltration and activation, thereby enabling a measurable antitumor immune response to be generated. Importantly, PDE-5 inhibitors downregulate expression of arginase (Arg), NO synthase and IL-4α in MDSCs, which resulted in restoration of cytotoxic activities of T cells (Serafini et al., 2006, J Exp med 203(12): 2691-702). Sildenafil therapy was reported by Meyer et al. (2011, Proc Natl Acad Sci 108(41): 17111-6) to delay tumor progression in melanoma bearing mice in association with decreased MDSC amounts and impaired immunosuppressive function. Moreover, the concentration of numerous inflammatory mediators (e.g., IL-1β, IL-6, VEGF, GM-CSF, MCP-1) was significantly diminished in melanoma lesions, indicating an anti-inflammatory effect of sildenafil.

There is clearly an important need to develop effective approaches for improving treatment of diseases such as cancer and infectious diseases for which therapeutic treatments are quite limited especially in advanced and chronic stages. It is especially the case for HBV-associated diseases due to the persistent nature of HBV, its high prevalence, the continuing transmission of HBV and the high incidence of cirrhosis and hepatocellular carcinomas in chronically HBV-infected patients. Combination therapies that combine two therapeutic agents that work by different mechanisms of action are described herein. The combination of the present invention combining immunotherapy and MDSC antagonist(s) aims at potentiating the patient's responses while inhibiting MDSCs generally involved in inhibition of T cell-mediated immunity. More specifically, the MDSC modulator will act to abrogate MDSC-mediated immunosuppressive activity, enabling the immunotherapeutic agent to enhance effective and specific immune responses. Moreover, combination therapy as disclosed herein may allow for lower doses than used in monotherapy, thereby reducing toxic side effects and/or increasing the therapeutic index of the individual entities. Combination therapy may also decrease the likelihood that resistance will develop.

### SUMMARY OF THE INVENTION

One aspect of the present invention relates to a combination comprising at least (a) a composition comprising a therapeutically effective amount of an immunotherapeutic product and at least (b) one or more MDSC modulator(s).

In one embodiment, the immunotherapeutic product composition comprises a plasmid or a viral vector. Preferably, said viral vector is obtained from a poxvirus or an adenovirus. Said poxvirus is preferably MVA. Said adenovirus is a human adenovirus selected from the group consisting of Ad2, Ad3, Ad4, Ad5, Ad7, Ad11, Ad19a, Ad24, Ad26, Ad34, Ad35, Ad48, Ad49 and Ad50 or a simian adenovirus selected from the group consisting of chimpanzee, gorilla, bonobo, cynomolgus macaque and rhesus macaque adenoviruses. A preferred immunotherapeutic product comprises a replication-defective adenovirus obtained from a human adenovirus of serotype 5 (Ad5) which is defective for E1 and/or E3 function(s).

In another embodiment, the immunotherapeutic product composition includes or encodes one or more antigen(s) such as cancer antigen(s) or antigen(s) originating from an infectious organism or associated with a disease or condition caused by an infectious organism. Said one or more antigens are selected from the group consisting of mucin antigens, HPV antigens, HCV antigens, HBV antigens, and Mtb antigens; and any combination thereof.

In a further embodiment, the immunotherapeutic product encodes at least one antigen originating from a hepatitis B virus (HBV) and preferably selected from the group consisting of HBV polymerase, HBc and HBs antigens. A preferred immunotherapeutic product encodes a fusion protein of HBc, pol and HBsAg, such as a fusion protein comprising an amino acid sequence that is at least 80% identical to SEQ ID NO: 8. Said HBV antigen fusion is preferably inserted in a replication-defective adenovirus in place of the E1 region and placed under the control of a promoter such as the CMV promoter.

In still a further embodiment, the one or more MDSC modulator(s) is/are capable of antagonizing the activity of phosphodiesterase subtype 5 (PDE-5). Said PDE5 inhibitor is preferably selected from the group consisting of avanafil, lodenafil, mirodenafil, sildenafil, actetildenafil, hydroxyacetildenafil, dimethylsildenafil, thiomethisosildenafil, tadalafil, vardenafil, udenafil, zaprinast, icariin, sulfoaildenafil and benzamidenafil.

In yet a further embodiment, the combination comprises a composition comprising from about 10′ vp to about 10¹² vp of an adenovirus-based immunotherapeutic product, and from about 10 mg to about 100 mg of a PDE5 inhibitor, each given in one or more dose(s) over an adequate period of time.

In an additional embodiment, the immunotherapeutic product composition and the MDSC modulator(s) may be administered concurrently, sequentially, in an interspersed manner or in any combination of these types of administration.

In another aspect, the invention provides a composition comprising an immunotherapeutic product for use in combination with one or more MDSC modulator(s) in an amount sufficient to treat or prevent a disease or a pathologic condition in a subject in need thereof. The immunotherapeutic product composition is preferably formulated for intramuscular, intradermal, transcutaneous, subcutaneous or intratumoral administration and the one or more MDSC modulator(s) is/are formulated for oral, sublingual or intravenous administration

In another embodiment, the combination is used for treating or preventing a disease characterized by MDSC-mediated immunosuppression or for treating a subject having a condition that would benefit from upregulation of the immune response. Said disease or pathologic condition is preferably selected from the group consisting of proliferative diseases, infectious diseases and acute or chronic inflammatory diseases. Said proliferative disease is preferably cancer and particularly a cancer selected from the group consisting of renal cancer, bladder cancer, prostate cancer, breast cancer, colorectal cancer, lung cancer, liver cancer, gastric cancer, pancreatic cancer, melanoma, ovarian cancer and glioblastoma, and especially metastatic ones. Said infectious diseases is preferably a viral infection associated with HPV (Human Papilloma Virus), HCV (Human hepatitis C Virus) or HBV virus or a bacterial infection associated with Mycobacterium.

In a further embodiment, the MDSC modulator therapy is administered more frequently than the immunotherapeutic product composition. In another embodiment, the immunotherapeutic product therapy and the MDSC modulator therapy overlap at least partially. In one aspect of this embodiment, administrations of the immunotherapeutic product and MDSC modulator(s) start at approximately the same time period. In another aspect, the MDSC modulator(s) is/are given to the subject before initiating administrations of the immunotherapeutic product, e.g. at least one week before initiating administration(s) of the immunotherapeutic product. In still another aspect, the administration(s) of the immunotherapeutic product composition is initiated before starting the MDSC modulator(s) therapy with a specific preference for administration of the MDSC modulator(s) which being initiated at the very end of the immunotherapeutic product administration(s) or very shortly after. A preferred regimen comprises a) 3 weekly subcutaneous or intramuscular administrations of about 10⁹ vp, about 10¹⁰ vp or about 10¹¹ vp of an adenovirus-based immunotherapeutic product composition and b) oral administrations of 10 to 100 mg of said MDSC modulator(s) given daily or every 2 days for at least one-month period therapy.

In another aspect is provided a PDE-5 inhibitor for use for treating or preventing an infectious disease, especially a chronic infection disease such as a chronic hepatitis B as well as a PDE5 inhibitor for use for reversing immunosuppression in a subject having a chronic infectious disease.

### BRIEF DESCRIPTION OF THE FIGURES OF THE INVENTION

FIG. 1: Detection of Adenovirus-specific T cells producing IFNγ.

The capacity of splenocytes to produce IFNγ after different in vitro stimulations was measured by an IFNγ-ELISpot assay at day 76 post AAV2/8-HBV injection. Vector-specific T cell responses were assessed using the Ad-specific peptide FAL. Results are shown as the number of spots per 10⁶ splenocytes. Each dot represents an individual mouse and thick line represents the mean value for each group (+/−SEM). The horizontal gray line represents the technical cutoff value (50 spots/10⁶ cells) above which values are considered as positive T cell responses and which was defined as described in the Material and Methods. For groups with a positive T cell response, incidence of mice per group is indicated on the graph.

FIG. 2: Detection of HBV-Core-specific and HBV-Polymerase-specific T cells producing IFNγ.

The capacity of splenocytes to produce IFNγ after different in vitro stimulations was measured by an IFNγ-ELISpot assay at day 76 post AAV2/8-HBV injection. HBV-specific responses were assessed using the HBV-Core peptides (A, Full pool Core), -polymerase specific peptide VSA (B) or N13F (C). Results are shown as the number of spots per 10⁶ splenocytes. Each dot represents an individual mouse and thick line represents mean values for each group (+/−SEM). The horizontal gray line represents the technical cutoff value (50 spots/10⁶ cells) above which values are considered as positive T cell responses. For groups with a positive T cell response, incidence of mice per group is indicated on the graph.

FIG. 3: Mean number of spots detected in responder mice (>50 spots/10⁶ splenocytes) for HBV-Core-specific and HBV-Polymerase-specific IFNγ responses.

The capacity of splenocytes to produce IFNγ after different in vitro stimulations was measured by an IFNγ-ELISpot assay at day 76 post AAV2/8-HBV injection. HBV-specific responses were assessed using the HBV-Core peptides (Full pool Core), -polymerase specific peptide VSA or N13F. Shown are mean values of responder mice (>50 spots/10⁶ cells) (mean, SEM). The horizontal gray line represents the technical cutoff value (50 spots/10⁶ cells) above which values are considered as positive T cell responses.

FIG. 4 A-F: Longitudinal evaluation of the anti-HBcAg level by ELISA.

Graphs A-F illustrate anti-HBc antibody levels determined respectively in individual mice of the six study groups 1-6. Individual mice are shown as thin line and the group mean titers as bold dotted black line. In all graphs the Y-axis indicates the anti-HBc levels (OD₄₅₀) and the X-axis the time (number of days post-AAV2/8-HBV injection). Time points of immunization (D36, D43, D50) are shown by a dashed line and the period of sildenafil treatment is shown by a horizontal bar on the top of the graph.

FIG. 5 A-B: Evaluation of serum HBsAg level by ELISA and HBV viral load by PCR.

Graph A illustrates the median level of HBsAg determined in the serum of groups of 10 mice (groups a to h) treated with sildenafil at 5 mg/kg/day (group b), at 20 mg/kg/day (group c), or at 80 mg/kg/day (group d) alone, or with AdTG18201 (group e) alone or with a combination of AdTG18201 and sildenafil (at 5, 20, or 80 mg/kg/day, groups f, g and h, respectively). The control group a is untreated. The Y-axis indicates the median HBsAg level (ng/mL) for each group and the X-axis the time (number of days post-AAV2/8-HBV injection). Graph B illustrates the median level of HBV DNA determined in the serum of the same groups of mice by PCR. The Y-axis indicates the median HBV DNA level (copies/mL) for each group of animals and the X-axis the time (number of days post-AAV2/8-HBV injection). For both graphs, time points of AdTG18201 immunization (D36, D43, D50) are shown by a dashed line and the period of sildenafil treatment (from D31 to 59) is shown by a horizontal bar on the X axis.

FIG. 6 A-B: Evaluation of the percentage of responding mice in terms for HBsAg level and HBV viral load.

Graph A illustrates the percentage of HBsAg responding mice in each group (a to h) of 10 mice as described in FIG. 5 legend. Graph B illustrates the percentage of HBV DNA responding mice in each group (a to h) of 10 mice as described in FIG. 5 legend.

FIG. 7: Evaluation of serum HBV-RNA level by RT-qPCR.

The graph illustrates the median level of HBV RNA determined in the serum of 6 mice per group (groups a, b, e and f) treated with sildenafil at 5 mg/kg/day (group b) or with AdTG18201 alone (group e) or with a combination of AdTG18201 and sildenafil (at 5 mg/kg/day, group f). The control group a is untreated. The Y-axis indicates the median HBV RNA level (log 10 copies/mL) and the X-axis the time (number of days post-AAV2/8-HBV injection). Time points of AdTG18201 immunization (D36, D43, D50) are shown by a dashed line and the period of sildenafil treatment (from D31 to 59) is shown by a horizontal bar on the X axis.

FIG. 8: illustrates the therapeutic effects of MVA vaccine (MVATG9931) in CT26 colon cancer tumor models.

BALB/c mice were intravenously (IV) injected (in caudal vein) with 2×10⁵ CT26-MUC1 cells. On day 2 and 9 after tumor challenge, mice were treated with 5×10⁷ pfu of MVATG9931 or Buffer. Sildenafil (Sildenafil citrate, Euromedex) was administered in drinking water from day 0 to 28 at a concentration of 0.52 mg/mL (corresponding to 80 mg/kg/day for a mouse of 20 g). The drinking bottle were replaced with freshly prepared sildenafil solution twice a week. Mice were weighed twice per week and sacrificed when reaching 10% weight loss. Overall survival rates represented as Kaplan-Meier curves. Animal experiments were conducted in compliance with EU directive 2010/63/EU.

### GENERAL DEFINITIONS

As used throughout the entire application, the terms “a” and “an” are used in the sense that they mean “at least one”, “at least a first”, “one or more” or “a plurality” of the referenced components or steps, unless the context clearly dictates otherwise. For example, the term “a cell” includes a plurality of cells, including mixtures thereof.

The term “one or more” refers to either one or a number above one (e.g. 2, 3, 4, 5, etc).

The term “and/or” wherever used herein includes the meaning of “and”, “or” and “all or any other combination of the elements connected by said term”.

The term “about” or “approximately” as used herein means within 20%, preferably within 10%, and more preferably within 5% of a given value or range.

When used to define products, compositions and methods, the term “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are open-ended and do not exclude additional, unrecited elements or method steps. Thus, a composition “comprises” the recited components when such components might be part of the final composition. “Consisting essentially of” means excluding other components or steps of any essential significance. Thus, a composition consisting essentially of the recited components would not exclude trace contaminants and pharmaceutically acceptable carriers. “Consisting of” means excluding more than trace elements of other components or steps.

The terms “polypeptide”, “peptide” and “protein” are used interchangeably to refer to polymers of amino acid residues comprising at least five amino acids covalently linked by peptide bonds. The polymer can be linear, branched or cyclic and may comprise naturally occurring and/or amino acid analogs and it may be interrupted by non-amino acids. No limitation is placed on the maximum number of amino acids comprised in a polypeptide. As a general indication, the term refers to both short polymers (typically designated in the art as peptide) and to longer polymers (typically designated in the art as polypeptide or protein). This term encompasses native polypeptides, modified polypeptides (also designated analogs), polypeptide fragments, polypeptide multimers (e.g. dimers), recombinant polypeptides, fusion polypeptides among others.

Within the context of the present invention, the terms “nucleic acid”, “nucleic acid molecule”, “polynucleotide”, “nucleic acid sequence” and “nucleotide sequence” are used interchangeably and define a polymer of at least 15 nucleotide residues (also called “nucleotides”) in either deoxyribonucleic acid (DNA) or ribonucleic acid (RNA) or mixed polyribo-polydeoxyribonucleotides. These terms encompass single or double-stranded, linear or circular, natural or synthetic, unmodified or modified versions thereof (e.g. genetically modified polynucleotides; optimized polynucleotides), sense or antisense polynucleotides, chimeric mixture (e.g. RNA-DNA hybrids). Exemplary DNA nucleic acids include without limitations, complementary DNA (cDNA), genomic DNA, plasmid DNA, DNA vector, viral DNA (e.g. viral genomes, viral vectors), oligonucleotides, probes, primers, satellite DNA, microsatellite DNA, coding DNA, non-coding DNA, antisense DNA, and any mixture thereof. Exemplary RNA nucleic acids include, without limitations, messenger RNA (mRNA), precursor messenger RNA (pre-mRNA), small interfering RNA (siRNA), short hairpin RNA (shRNA), microRNA (miRNA), RNA vector, viral RNA, guide RNA (gRNA), antisense RNA, coding RNA, non-coding RNA, antisense RNA, satellite RNA, small cytoplasmic RNA, small nuclear RNA. Polynucleotides described herein may be synthesized by standard methods known in the art, e.g., by use of an automated DNA synthesizer (such as those that are commercially available from Biosearch, Applied Biosystems, etc.) or obtained from a naturally occurring source (e.g. a genome, cDNA, etc.) or a artificial source (such as a commercially available library, a plasmid, etc.) using molecular biology techniques well known in the art (e.g. cloning, PCR, etc).

The term “analog” as used herein to qualify a polypeptide or a nucleic acid refers to one or more modification(s) with respect to the native counterpart. Any modification(s) can be envisaged, including substitution, insertion and/or deletion of one or more nucleotide/amino acid residue(s). When several mutations are contemplated, they can concern consecutive residues and/or non-consecutive residues. Mutation(s) can be generated by a number of ways known to those skilled in the art, such as site-directed mutagenesis (e.g. using the Sculptor™ in vitro mutagenesis system of Amersham, Les Ullis, France), PCR mutagenesis, DNA shuffling and by synthetic techniques. Preferred are analogs that retain a degree of sequence identity of at least 80%, preferably at least 85%, more preferably at least 90%, and even more preferably at least 98% identity with the sequence of the native polypeptide or nucleic acid counterpart or a portion thereof of at least 30 residues. For illustrative purposes, “at least 80% identity” means 80%, 81%, 82%, 83%, 84%, 85%, 86%, 87%, 88%, 89%, 90%, 91%, 92%, 93%, 94%, 95%, 96%, 97%, 98%, 99% or 100%. The percentage of identity between two sequences is a function of the number of identical positions shared by the sequences, taking into account the number of gaps which need to be introduced for optimal global alignment and the length of each gap. Various computer programs and mathematical algorithms are available in the art to determine the percentage of identity between amino acid sequences after optimal global alignment, such as for example the algorithm of Needleman et Wunsch. J. Mol. Biol. 48, 443-453, 1970, the Blast program available at NCBI or ALIGN in Atlas of Protein Sequence and Structure (Dayhoffed, 1981, Suppl., 3: 482-9). Programs for determining identity between nucleotide sequences after optimal global alignment are also available in specialized data base (e.g. Genbank, the Wisconsin Sequence Analysis Package, BESTFIT, FASTA and GAP programs, and the needle software available from ebi.ac.uk worldwide under the name «Align»).

The term “native” as used herein refers to the original source of a component (e.g. a polypeptide, nucleic acid, vector, virus, etc.) meaning that the component can be obtained, found or isolated from such a source.

The term “obtained from”, “originating from” or “derived from” is used to identify the original source of a component but is not meant to limit the method by which the component is made which can be, for example, by chemical synthesis or recombinant means.

As used herein, the term “isolated” refers to a component (e.g. a polypeptide, polynucleotide, vector, small molecule, etc.), that is removed from its natural environment (i.e. separated from at least one other component(s) with which it is naturally associated or found in nature). An isolated component refers to a component that is maintained in a heterologous context or purified (partially or substantially). For example, a nucleic acid molecule is isolated when it is separated of sequences normally associated with it in nature (e.g. dissociated from a chromosome or a genome) but it can be associated with heterologous sequences (e.g. within a recombinant vector). A synthetic component is isolated by nature.

As used herein, the term “host cell” should be understood broadly without any limitation concerning particular organization in tissue, organ, or isolated cells. Such cells may be of a unique type of cells or a group of different types of cells such as cultured cell lines, primary cells and dividing cells. In the context of the invention, “host cells” include prokaryotic cells, lower eukaryotic cells such as yeast, and higher eukaryotic cells with a specific preference for mammalian (e.g. human or non-human) cells. This term also encompasses producer cells capable of producing the immunotherapeutic product for use in the combination described herein as well as cells which are or has been the recipient of such a combination and progeny thereof.

The term “subject” generally refers to a living organism for whom any product and method of the invention is needed or may be beneficial. In the context of the invention, the subject is a mammal, particularly a mammal selected from the group consisting of domestic animals, farm animals, sport animals, and primates. Preferably, the subject is a human who has been diagnosed as being or at risk of having a pathological condition such as an infectious disease (e.g. a chronic infectious disease such as hepatitis B) or a proliferative disease (e.g. cancer). The terms “subject” and “patient” may be used interchangeably when referring to a human organism and encompasses male and female as well as newborn, infant, young adult, adult and eldery. The subject may also be naïve of treatment or under conventional treatment with respect to the targeted pathological condition (e.g. NUC (nucleos(t)ide analog) treatment for a hepatitis B patient).

The term “treatment” (and any form of treatment such as “treating”, “treat”) as used herein encompasses prophylaxis (e.g. preventive measure in a subject at risk of having the pathological condition) and/or therapy (e.g. in a subject diagnosed as having the pathological condition), optionally in association with conventional therapeutic modalities. The result of the treatment is to slow down, cure, ameliorate or control the progression of the targeted pathological condition. For example, a subject is successfully treated for an HBV infection or associated diseases if after administration of the combination as described herein, the subject shows an observable improvement of its clinical status.

The term “administering” (or any form of administration such as “administered”) as used herein refers to the delivery to a subject of at least one of the component of the combination of the invention.

### DETAILED DESCRIPTION OF THE INVENTION

In a first aspect the present invention provides a combination comprising at least (a) a composition comprising a therapeutically effective amount of an immunotherapeutic product and (b) one or more MDSC modulator(s).

The term “combination” as used herein refers to any arrangement possible of the at least two entities that are subject of the present invention and described herein (i.e. a composition comprising an immunotherapeutic product and one or more MDSC modulator(s)). Preferably, the combination is synergistic providing higher efficacy than each entity alone. For example, the immune response observed with the combination of the invention is greater or intensified in any way (duration, magnitude, intensity, etc) when compared to the same immune response measured with each entity alone under the same conditions. The combination of the invention is particularly useful for improving a CD8+ and/or a CD4+ T cell-mediated immune response in a subject exposed to the combination especially against one or more antigen(s) carried by said immunotherapeutic product.

A “therapeutically effective amount” corresponds to the amount of immunotherapeutic product which is sufficiently effective to produce or contribute to a desired therapeutic effect in combination with one or more MDSC modulator(s). Examples of a desired therapeutic effect is enhancing an immune response, slowing, delaying or stabilizing the development of the targeted pathological condition; or amelioration of one or more symptoms. An effective amount may be given as a single dose or as a series of doses. Such a therapeutically effective amount may vary as a function of various parameters such as the agent itself (kind of immunotherapeutic product and MDSC modulator), the pathological condition to be treated (e.g. nature and severity of symptoms, kind of concurrent treatment, the need for prevention or therapy, etc), the subject (age, weight, its ability to respond to the treatment), and/or the mode of administration; etc.

The term “immunotherapeutic product” as used herein refers to a product comprising one or more antigen(s) which is expected to induce or activate an immune response—whether specific or non-specific; humoral or cellular—when delivered appropriately to a subject, according to the modalities described herein.

The term “MDSC modulator” as used herein, refers to a component or a group of components capable of directly or indirectly modulating MDSC's activity in a positive or negative way. In accordance with this invention, the one or more MDSC modulator(s) exert(s) an antagonist function (i.e. being capable of antagonizing, at least partially, the MDSC's inhibitory signal, in particular in an inflammatory environment). For illustrative purposes, the action of such one or more MDSC modulator(s) may independently be at different levels of the MDSC's signalling pathway, e.g. by down-regulating the MDSC's function, activation, proliferation, recruitment to inflammatory sites and/or depleting MDSCs, and/or favouring their differentiation into non-immunosuppressive cells, etc. The mechanism of action of such one or more MDSC modulator(s) may be by direct interaction with MDSCs (e.g. through interaction with a receptor present at the MDSC surface or a ligand thereof) or by indirect interaction (e.g. through interaction with biological substance(s) involved in MDSC signalling pathway). For example, such one or more MDSC modulator(s) may downregulate the production of reactive oxygen species, peroxynitrites, arginase and/or nitrous oxide; and/or inhibit the enzymatic metabolism involved in the production of at least one of these metabolites (e.g. nitrous oxide synthase 2 (NOS2), arginase, etc); and/or inhibit one or more of the cytokines such as IFN-γ IL-4 and IL-13 that are involved in MDSC's activation or recruitment). A reduction of MDSC's immunosuppressive activity is preferably at least 20% (25%, 30%, 35%, 40%, 45%, 50%, 55%, 60%, 65%, 70%, 75%, 80%, 85%, 90% or more). The immunosuppressive activity of MDSC can be determined for example by histochemistry methods (detection of markers such as ARG-1, iNOS, IDO, STAT-3, etc., by FACS) or other conventional assays (e.g. inhibition of T cell proliferation in vitro, etc.). A reduction of immunosuppressive activity can also be measured by an improvement in immune responses, and particularly T cell and B cell responses (e.g. a measurement of IFNg producing T cells as described in the Example section and/or measurement of antibody induction), or as measured by improvements in survival, reductions in tumor or infectious disease burden.

Immunotherapeutic Product

Any type of immunotherapeutic product can be used in the context of the invention including, but not limited to, cell-based products, peptide or polypeptide-based products, microorganism-based products and vector-based products.

Cell based products typically rely on cells (e.g. cancer cells, immune cells such as dendritic cells and stem cells) collected from a subject, treated and/or reprogramed in vitro to be more amenable to the subject's immune system before being reinfused into a subject's bloodstream.

Polypeptide-based products can be generated by recombinant or synthetic means. Numerous polypeptide-based products are currently developed. One may cite, for example, the liposomal vaccine Stimuvax® which incorporates lipopeptides generated from the mucin 1 (MUC1) glycoprotein and showed beneficial effects in some subgroups of patients with advanced non-small cell lung cancer (NSCLC); Newax E75 developed by Galena and Genentech for breast cancer; SL-701, a synthetic multipeptide vaccine developed by Stemline Therapeutics for treating glioma brain tumors; and monoclonal antibodies that are now conventionally used in clinics to attack specific types of diseased cells (e.g. the anti-CD20 rituximab approved for treatment of non-Hodgkins lymphomas, trastuzumab for the treatment of breast cancer with HER2/neu overexpression and bevacizumab that target VEGF and can be used as antiangiogenic cancer therapy).

Microorganism-based immunotherapeutic products typically employ avirulent or attenuated microorganisms which optionally have been engineered for expressing polypeptides of interest. Well-known examples of suitable microorganisms include without limitation bacterium (e.g. Mycobacterium; Lactobacillus (e.g. Lactococcus lactis); Listeria (e.g. Listeria monocytogenes) Salmonella and Pseudomona) and yeast (e.g. Saccharomyces cerevisiae, Schizosaccharomyces pombe, Pichia pastoris). A suitable bacterium-based immunotherapeutic product is Mycobacterium bovis (BCG) widely used for treating bladder cancer.

Vector Based Immunotherapeutic Product

In one embodiment, the immunotherapeutic product for use in this invention is a vector-based immunotherapeutic product (or vectorized immunotherapeutic product).

The term “vector” as used herein refers to a vehicle that contains the elements necessary to allow delivery, propagation and/or expression of biological substance(s) within a host cell or subject. This term encompasses extrachromosomal vectors (e.g. that remain in the cell cytosol or nucleus) and integration vectors (e.g. designed to integrate into the cell genome) as well as cloning vectors, shuttle vectors (e.g. functioning in both prokaryotic and/or eukaryotic hosts), transfer vectors (e.g. for transferring nucleic acid molecule(s) in a viral genome) and expression vectors for expression in various host cells or organisms. For the purpose of the invention, the vectors may be of naturally occurring genetic sources, synthetic or artificial, or some combination of natural and artificial genetic elements.

In the context of the invention, the term “vector” has to be understood broadly as including DNA and RNA vectors as well as plasmid and viral vectors. Typically, such vectors are commercially available (e.g. in Invitrogen, Stratagene, Amersham Biosciences, Promega, etc.) or available from depositary institutions such as the American Type Culture Collection (ATCC, Rockville, Md.) or have been the subject of numerous publications describing their sequence, organization and methods of producing, allowing the artisan to apply them.

A “plasmid” as used herein refers to a replicable DNA construct. Usually plasmid vectors contain selectable marker genes that allow host cells carrying the plasmid vector to be selected for or against in the presence of a corresponding selective drug. A variety of positive and negative selectable marker genes are known in the art. By way of illustration, an antibiotic resistance gene can be used as a positive selectable marker gene that allows selection of the plasmid-containing cells in the presence of the corresponding antibiotic. Representative examples of suitable plasmid vectors include, without limitation, pREP4, pCEP4 (Invitrogene), pCI (Promega), pCDM8 (Seed, 1987, Nature 329: 840), pMT2PC (Kaufman et al., 1987, EMBO J. 6: 187-95), pVAX (Invitrogen) and pgWiz (Gene Therapy System Inc).

The term “viral vector” as used herein refers to a vector that includes at least one element of a virus genome allowing packaging into a viral particle. This term has to be understood broadly as including nucleic acid vector (RNA or DNA) as well as viral particles generated thereof. In accordance with the present invention, the viral vector can be live, inactivated, attenuated, killed, oncolytic, etc., and it can also be replication-competent, replication-selective (e.g. engineered to replicate better or selectively in specific host cells), or replication-defective (which means that it cannot replicate to any significant extent in non-permissive cells due to partial or total deletion or inactivation of regions critical to viral replication). Replication-defective viral vectors typically require for propagation, permissive host cells which bring up or complement the missing/impaired functions. The terms “virus”, “virions” and viral particles” are used interchangeably to refer to viral particles that are formed when the viral vector genome is delivered into an appropriate cell or cell line according to suitable conditions allowing the generation of infectious viral particles. The term “infectious” refers to the ability of a viral vector to infect and enter into a host cell or subject.

Viral vectors can be engineered from a variety of viruses and in particular from the group of viruses consisting of adenovirus, poxvirus, adenovirus-associated virus (AAV), herpes virus (HSV), measles virus, foamy virus, alphavirus, vesicular stomatis virus, Newcastle disease virus, picorna virus, Sindbis virus, lentivirus, etc. One may use either wild-type strains as well as modified versions thereof. Modification(s) can be within endogenous viral genes (e.g. coding and/or regulatory sequences) and/or within intergenic regions. Moreover, modification(s) can be silent or not (e.g. resulting in a modified viral gene product). Modification(s) can be made in a number of ways known to those skilled in the art using conventional molecular biology techniques. Desirably, the modifications encompassed by the present invention affect, for example, virulence, toxicity, pathogenicity or replication of the virus compared to a virus without such modification, but do not completely impair infection and production at least in permissive cells. Preferably, the immunotherapeutic product comprised in the combination of the invention comprises a replication-defective viral vector.

In one embodiment, the immunotherapeutic product for use in combination with the MDSC modulator(s) according to the present invention is obtained from a poxvirus. As used herein the term “poxvirus” refers to a virus belonging to the Poxviridae family with a preference for the Chordopoxvirinae subfamily which includes several genus such as Orthopoxvirus, Capripoxvirus, Avipoxvirus, Parapoxvirus, Leporipoxvirus and Suipoxvirus. Orthopoxviruses are preferred in the context of the present invention and even more vaccinia virus (VV) species. Suitable poxvirus for use herein include, without limitations, Western Reserve (WR), Copenhagen (Goebel et al., 1990, Virol. 179: 247; Johnson et al., 1993, Virol. 196: 381), Wyeth and MVA vaccinia virus as well as those described for example in U.S. Pat. No. 5,494,807 (describing NYVAC), WO2009/065547 (describing TK- and F2L-defective VV) and WO2009/065546 (describing TK- and 14L-defective VV). The genomic sequences and encoded viral polypeptides are available in specialized databanks such as Genebank (e.g. accession numbers NC_006998, M35027, NC_005309, U94848 providing WR, Copenhagen, Canarypoxvirus and MVA genomic sequences). A particularly appropriate viral vector for use in the context of the present invention is MVA due to its highly attenuated phenotype (Mayr et al., 1975, Infection 3: 6-14; Sutter and Moss, 1992, Proc. Natl. Acad. Sci. USA 89: 10847-51), a more pronounced IFN-type 1 response generated upon infection compared to non-attenuated vectors and availability of the sequence of its genome in the literature (Antoine et al., 1998, Virol. 244: 365-96 and Genbank accession number U94848).

In another embodiment, the immunotherapeutic product for use in the present invention is obtained from a paramyxoviridae and especially from a morbillivirus such as measles. Various attenuated strains are available in the art, such as the Edmonston A and B strains (Griffin et al., 2001, Field's in Virology, 1401-1441), the Schwarz strain (Schwarz A, 1962, Am J Dis Child, 103: 216), the 5-191 or C-47 strains (Zhang et al., 2009, J Med Virol. 81 (8): 1477). One may also use recombinant Newcastle Disease Virus (NDV) (Bukreyev and Collins, 2008, Curr Opin Mol Ther 10: 46-55) with a specific preference for attenuated strains such as MTH-68 that was already used in cancer patients (Csatary et al., 1999, Anti Cancer Res 19: 635-8) and NDV-HUJ, which showed promising results in glioblastoma patients (isracast.com Mar. 1, 2006).

In still another and particularly preferred embodiment, the immunotherapeutic product for use in the combination according to the present invention is obtained from an adenovirus. The term “adenovirus” (or Ad) refers to a group of viruses belonging to the Adenoviridae family. Generally speaking, adenoviruses are non-enveloped and their genome consists of a single molecule of linear, double stranded DNA that codes for more than 30 proteins including the regulatory early proteins participating in the replication and transcription of the viral DNA which are distributed in 4 regions designated E1 to E4 (E denoting “early”) dispersed in the adenoviral genome and the late (L) structural proteins (see e.g. Evans and Hearing, 2002, in “Adenoviral Vectors for Gene Therapy” pp 39-70, eds. Elsevier Science). E1, E2 and E4 are essential to the viral replication whereas E3 is dispensable and appears to be responsible for inhibition of the host's immune response in the course of adenovirus infection.

Adenoviral vectors for use herein can be obtained from a variety of human or animal adenoviruses and any serotype can be employed including those of rare serotypes. It can also be a chimeric adenovirus (WO2005/001103). One of skill will recognize that elements derived from multiple serotypes can be combined in a single adenovirus. Representative examples of suitable human adenoviruses include subgenus C (e.g. Ad2 Ad5 and Ad6), subgenus B (e.g. Ad3, Ad7, Ad11, Ad14, Ad34, Ad35 and Ad50), subgenus D (e.g. Ad19a, Ad24, Ad26, Ad48 and Ad49) and subgenus E (Ad4). Simian Ad are also appropriate in the context of the invention, especially to overcome human Ad pre-immunity. Such simian Ad can originate from a variety of monkeys (e.g. chimpanzee, gorilla, bonobo, cynomolgus macaque and rhesus macaque). One may cite more particularly chimp Ad such as AdCh3 (Peruzzi et al., 2009, Vaccine 27: 1293-300) and AdCh63 (Dudareva et al, 2009, Vaccine 27: 3501-4) and gorilla Ad (see e.g. WO2013/052799; WO2013/052811 and WO2013/052832) as well as any of those described in the art (see for example, WO2010/086189; WO2009/105084; WO2009/073104; WO2009/073103; WO2005/071093; and WO03/046124).

Preferably, the adenovirus employed in this invention is replication-defective, e.g. by total or partial deletion of E1 region. An appropriate E1 deletion extends from approximately positions 459 to 3510 by reference to the Ad5 sequence disclosed in the Gen Bank under the accession number M 73260 and in Chroboczek et al. (1992, Virol. 186: 280-5). Additional modification(s) may be carried out in the Ad genome (e.g. deletion of all or part of other essential E2 and/or E4 regions as described in WO94/28152; Lusky et al, 1998, J. Virol 72: 2022). In addition, the non-essential E3 region can also be mutated or deleted (at least partially). In a preferred embodiment, the immunotherapeutic product comprises a replication-defective adenovirus obtained from a human adenovirus of serotype (Ad5) which is defective for E1 and/or E3 function(s).

The present invention also encompasses immunotherapeutic products complexed to lipids or polymers (e.g. polyethylene glycol) to form particulate structures such as liposomes, lipoplexes or nanoparticles as well as targeted ones modified to allow preferential targeting to a specific host cell. Targeting can be carried out through genetic means (e.g. by genetically inserting a ligand capable of recognizing and binding to a cellular and surface-exposed component into a polypeptide present on the surface of the virus) or by chemical means (e.g. by modifying a viral surface envelope). Examples of suitable ligands include antibodies or fragments thereof directed to cell-specific, tissue-specific and pathogen-associated markers.

Recombinant Vectorized Immunotherapeutic Product

In most embodiments of the present invention, the immunotherapeutic product for use herein has been engineered to deliver in situ one or more polypeptide(s) of interest (i.e. a recombinant plasmid or viral vector). Such one or more polypeptide(s) of therapeutic interest are selected to compensate for pathological symptoms, e.g. by acting to limit or remove harmful cells from the body (e.g. a suicide gene product) and/or by acting as target polypeptide against which it is desired to elicit an immune response (e.g. an antigen) or by improving the host's immune system (e.g. a cytokine). Such polypeptides can be obtained from a natural source—of mammal origin (e.g. human) or not (e.g. from a pathogen)—or be altered in lab (so as to include suitable sequence modification(s)) and can be produced by synthetic means or by a biological process (e.g. recombinantly produced).

In a preferred embodiment of the invention, the immunotherapeutic product composition includes or encodes one or more antigen(s).

The term “antigen” generally refers to a substance that is recognized and selectively bound by an antibody or by a T cell antigen receptor, in order to trigger an immune response. It is contemplated that the term antigen encompasses native antigen as well as fragment (e.g. epitopes, immunogenic domains, etc) and analog thereof, provided that such fragment or analog is capable of being the target of an immune response. An antigen can be as small as a single epitope or a single immunogenic domain or can be larger to include multiple epitopes or immunogenic domains. As such, the size of an antigen can be as small as about 8-11 amino acids and as large as a full-length protein, a multimer, a fusion protein, a chimeric protein, a whole cell, a whole microorganism, or any portions thereof. For example, an antigen can contain multiple different immunogenic domains and immunogenic domains can contain one or multiple epitope(s).

An “epitope” is defined herein as the minimal part of an antigen that is recognized by components of the immune system and that is sufficient to elicit an immune response when provided to the immune system in the context of appropriate costimulatory signals and/or activated cells of the immune system. Those of skill in the art will recognize that T cell epitopes are different in size and composition from B cell or antibody epitopes, and that epitopes presented through the Class I MHC pathway differ in size and structural attributes from epitopes presented through the Class II MHC pathway. For example, T cell epitopes presented by Class I MHC molecules are typically between 8 and 11 amino acids in length, whereas epitopes presented by Class II MHC molecules are less restricted in length and may be up to 25 amino acids or longer. Epitopes need not be linear sequences (constituted of consecutive amino acid residues within an antigen) and conformational epitopes involving nonconsecutive amino acid residues are also encompassed by the present invention.

An “immunogenic domain” of a given antigen can be any portion, fragment of an antigen that contains at least one epitope that can act as an immunogen when administered to a subject. Immunogenic domains may include one or more B cell epitope(s) or one or more T cell epitope(s) or both B and T cell epitope(s) and capable of raising an immune response, preferably, a humoral or cell response that can be antigen-specific or innate. Immunogenic domains are usually between 15 to 100 amino acid residues long (e.g. from 20 to 80, from 25 to 65 amino acid residues).

Typically, the one or more antigen(s) for expression by the immunotherapeutic product is selected in connection with the pathological condition to treat. In one embodiment, the antigen elicits a cell-mediated immune response, including a CD4 T cell response (e.g., Th1, Th2 and/or Th17) and/or a CD8+ T cell response (e.g., a CTL response). A vast variety of direct or indirect biological assays are available in the art to evaluate the immunogenic nature of an antigen either in vivo (animal or human being), or in vitro (e.g. in a biological sample) as described herein.

In one embodiment, the one or more antigen(s) to be expressed by the immunotherapeutic product comprise(s) a cancer antigen. As used herein, the term “cancer antigen” refers to a polypeptide that is associated with and/or serve as markers for cancers. Cancer antigens encompass various categories of polypeptides, e.g. those which are normally silent (i.e. not expressed) in normal cells, those that are expressed only at low levels or at certain stages of differentiation and those that are temporally expressed such as embryonic and foetal antigens as well as those resulting from mutation of cellular genes, such as oncogenes (e.g. activated ras oncogene), proto-oncogenes (e.g. ErbB family), or proteins resulting from chromosomal translocations. The cancer antigens also encompass antigens encoded by pathogenic organisms (bacteria, viruses, parasites, fungi, viroids or prions) that are capable of inducing a malignant condition in a subject such as RNA and DNA tumor viruses (e.g. HPV, HCV, HBV, etc) and bacteria (e.g. Helicobacter pilori).

Advantageously, the immunotherapeutic product composition encodes one or more cancer antigen(s) associated with brain (e.g. glioblastoma), hepatocarcinoma, breast, pancreas or colorectal cancer.

Some non-limiting examples of suitable cancer antigens for use herein include, without limitation, MART-1/Melan-A (Kawakami et al., 1994, J. Exp. Med. 180: 347-52), gp100 (Kawakami et al., 1992, Proc. Natl. Acad. Sci. USA 91: 6458-62), Dipeptidyl peptidase IV (DPPIV), adenosine deaminase-binding protein (ADAbp), cyclophilin b, Colorectal associated antigen (CRC)-0017-1A/GA733, Carcinoembryonic Antigen (CEA) and its immunogenic epitopes CAP-1 and CAP-2 (GenBank Accession No. M29540), etv6, aml1, Prostate Specific Antigen (PSA) and its immunogenic epitopes (Xue et al., 1997, The Prostate 30: 73-8), prostate-specific membrane antigen (PSMA) (Israeli et al., 1993, Cancer Res. 53: 227-30), T-cell receptor/CD3-zeta chain, BRCA-family of tumor antigens (U.S. Pat. No. 5,747,282), MAGE-family of tumor antigens (see e.g., U.S. Pat. No. 5,750,395), GAGE-family of tumor antigens (U.S. Pat. No. 5,648,226), BAGE, RAGE, LAGE-1, NAG, GnT-V, MUM-1, CDK4, tyrosinase (Kwon et al., 1987, Proc. Natl. Acad. Sci. USA 84: 7473-7), MUC family (such as MUC1, MUC2, MUC16, etc.; see e.g. Jerome et al., 1993, J. Immunol. 151: 1654-62; U.S. Pat. No. 6,054,438; WO98/04727; and WO98/37095), mutated Ras oncoprotein (U.S. Pat. Nos. 7,465,454 and 7,563,447), normal and mutated p53 oncoproteins (Hollstein et al., 1994, Nucleic Acids Res. 22: 3551-5), HER2/neu, RCAS1, alpha-fetoprotein, E-cadherin, alpha-catenin, beta-catenin and gamma-catenin, NY-ESO-1 (Chen et al., 1997, Proc. Natl. Acad. Sci. USA 94: 1914-8), cdc27, adenomatous polyposis coli protein (APC), Smad family of cancer antigens, SSX family of cancer antigens and c-erbB-2 as well as viral antigens originating from oncogenic pathogenic organism as described hereinafter. A preferred embodiment is directed to an immunotherapeutic product expressing MUC1.

Alternatively or in combination with the cancer antigens embodiment, the immunotherapeutic product includes or encodes one or more antigen(s) originating from an infectious organism or associated with a disease or condition caused by an infectious organism. Such antigens include, but are not limited to, viral antigens, fungal antigens, bacterial antigens, parasitic antigens and protozoan antigens.

In one aspect of this embodiment, the immunotherapeutic product composition contains or expresses viral antigen(s). Such viral antigen(s) may originate from a vast variety of viruses including, among many others, coronaviruses, coxsackie viruses, flaviviruses, hepadnaviruses, hepatitis viruses, influenza viruses, lentiviruses, measles viruses, mumps viruses, myxoviruses, orthomyxoviruses, papilloma viruses, parainfluenza viruses, paramyxoviruses, parvoviruses, picornaviruses, rabies viruses, respiratory syncytial viruses (RSV), rhabdoviruses, rubella viruses, togaviruses, and varicella viruses. Preferably, the viral antigens to be expressed originate from hepatitis C virus (HCV), hepatitis B virus (HBV) and human papillomavirus (HPV) such as HPV-16 and HPV-18.

In another aspect, the immunotherapeutic product contains or expresses bacterial antigen(s) or antigen(s) from another infectious organism. Representative examples of such infectious organisms include, without limitation, Enterobacteriaceae (Escherichia), Leishmania, Mycobacterium (Mycobacterium tuberculosis (Mtb); Mycobacterium bovis, Mycobacterium leprae), Mycoplasma, Pneumococcus, Pneumocystis, Salmonella, Pseudomonas (e.g. Pseudomonas aeruginosa), Staphylococcus, Streptococcus, Toxoplasma, Vibriocholerae, Helicobacter (Helicobacter pylori) and Plasmodium (e.g. Plasmodium falciparum).

Other antigens suitable for use in this invention are marker antigens (beta-galactosidase, luciferase, green fluorescent proteins, etc.).

The present invention also encompasses immunotherapeutic products comprising/expressing several polypeptides of interest, e.g. at least two antigens, at least one antigen and one cytokine, at least two antigens and one cytokine, etc.

A preferred immunotherapeutic product composition comprised in the combination of the invention comprises or encodes one or more antigens of interest selected from the group consisting of mucin antigens (e.g. MUC-1); HPV antigens (e.g. the non-oncogenic E6 and E7 antigens described in WO99/03885); HCV antigens (e.g. the non-structural antigens NS3, NS4 and/or NS5 described in WO2004/111082); HBV antigens (in particular any of the core, polymerase and HBs antigens described in WO2011/015656 and WO2013/007772); and Mtb antigens (any of those described in WO2014/009438); and any combination thereof.

Some embodiments also contemplate the expression from the same vector of antigen(s) (e.g. human MUC1 or viral HPV E6 and E7) and cytokine(s) (e.g. human IL-2).

In still another embodiment, the polypeptide(s) of interest comprised or encoded by the immunotherapeutic product may be in the form of a fusion protein. The term “fusion” or “fusion protein” as used herein refers to the combination of two or more polypeptides/peptides in a single polypeptide chain. Preferably, the fusion is performed by genetic means, i.e. by fusing in frame the nucleotide sequences encoding each of said polypeptides/peptides. By “fused in frame”, it is meant that the expression of the fused coding sequences results in a single protein without any translational terminator between each of the fused polypeptides/peptides. The fusion can be direct (i.e. without any additional amino acid residues in between) or through a linker (e.g. 3 to 30 amino acids long peptide composed of amino acid residues such as glycine, serine, threonine, asparagine, alanine and/or proline). It is within the reach of the skilled person to define accordingly the need and location of the translation-mediating regulatory elements (e.g. the initiator Met and codon STOP).

Exemplary fusions for use herein are fusions of two or more antigens (or fragments or analogs thereof) or fusions of antigen(s) with polypeptides capable of enhancing immunogenicity. Such polypeptides have been described in the literature and include, without limitation, calreticulin (Cheng et al., 2001, J. Clin. Invest. 108: 669), Mycobacterium tuberculosis heat shock protein 70 (HSP70) (Chen et al., 2000, Cancer Res. 60: 1035), ubiquitin (Rodriguez et al., 1997, J. Virol. 71: 8497), bacterial toxin such as the translocation domain of Pseudomonas aeruginosa exotoxin A (ETA(dIII)) (Hung et al., 2001 Cancer Res. 61: 3698) as well as such as T_{H} Pan-Dr epitope (Sidney et al., 1994, Immunity 1: 751), pstS1 GCG epitope (Vordermeier et al., 1992, Eur. J. Immunol. 22: 2631), tetanus toxoid P2TT (Panina-Bordignon et al., 1989, Eur. J. Immunol. 19: 2237) and P30TT (Demotz et al., 1993, Eur. J. Immunol. 23: 425) peptides, and influenza epitope (Lamb et al., 1982, Nature 300: 66; Rothbard et al., 1989, Int. Immunol. 1: 479).

In the context of this invention, the polypeptide(s) of interest to be expressed by the immunotherapeutic product may include specific structural features that are useful to improve its/their cloning, synthesis, processing, stability, solubility and/or efficacy. For example, membrane anchorage may be useful to improve MHC class I and/or MHC class II presentation. Membrane presentation can be achieved by incorporating in the polypeptide of interest a membrane-anchoring sequence and a secretory sequence (i.e. a signal peptide) if the native polypeptide lacks it. Briefly, signal peptides usually comprise 15 to 35 essentially hydrophobic amino acids which are then removed by a specific ER (endoplasmic reticulum)-located endopeptidase to give the mature polypeptide. Trans-membrane peptides are also highly hydrophobic in nature and serve to anchor the polypeptides within cell membrane. Appropriate trans-membrane and/or signal peptides are known in the art. They may be obtained from cellular or viral polypeptides such as those of immunoglobulins, tissue plasminogen activator, insulin, rabies glycoprotein, the HIV virus envelope glycoprotein or the measles virus F protein or may be synthetic. Preferably, the secretory sequence is inserted at the N-terminus of the polypeptide downstream of the codon for initiation of translation and the membrane-anchoring sequence at the C-terminus, preferably immediately upstream of the stop codon.

HBV-Targeted Immunotherapeutic Product

In a preferred embodiment, the immunotherapeutic product encodes one or more antigen(s) originating from a hepatitis B virus, and more preferably from a human hepatitis B virus (HBV). As used herein, “hepatitis B virus” refers to any member of the Hepadnaviridae (see e.g. Ganem and Schneider in Hepadnaviridae (2001) “The viruses and their replication”, pp 2923-2969, Knipe D M et al, eds. Fields Virology, 4th ed. Philadelphia, Lippincott Williams & Wilkins or subsequent edition). Typically, Hepadnaviruses are small enveloped hepatotropic DNA viruses having a partially double-stranded, circular DNA of approximately 3,200 nucleotides with a compact gene organization. More specifically, the HBV genome contains 4 overlapping open reading frames (ORFs), C, S, P and X. The C ORF encodes the core protein (or HBc) constitutive of the nucleocapsid, the S ORF the envelop proteins, the P ORF the viral polymerase and the X ORF a protein known as the X protein which is thought to be a transcriptional activator.

In accordance with the present invention, each of the one or more HBV antigens encoded by the immunotherapeutic product described herein may independently originate from any HBV that can be found, isolated, obtained from a source in nature, whatever its genotype and serotype. HBV are classified into eight genotypes (A to H) divided into nine serotypes (ayw1, ayw2, ayw3, ayw4, ayr, adw2, adw4, adrq+ and adqr−) according to HBsAg-associated serology (see review by Mamum-Al Mahtab et al., 2008, Hepatobiliary Pancrease Dis Int 5: 457-64; Schaeffer, 2007, World Gastroenterol. 7: 14). The genotypes show distinct geographic distribution and clinical outcome and differ each other by at least 8% of their sequence. Each genotype and serotype encompasses different HBV strains and isolates. An isolate corresponds to a specific virus isolated from a particular source of HBV (e.g. a patient sample or other biological HBV reservoir) whereas a strain encompasses various isolates which are very close each other in terms of genomic sequences. A vast number of HBV are described in the literature and their genomic sequence and encoded amino acid sequences can be found in specialized data banks (e.g. Genbank).

In one embodiment, the one or more HBV antigen(s) encoded by the immunotherapeutic product composition comprised in the combination of the present invention is/are selected from the group consisting of HBV polymerase (pol), HBc (core) and HBsAg polypeptides (encompassing fragments and analogs thereof as mentioned above) and any combination thereof. In accordance with the purpose of the present invention, such encoded HBV antigen(s) can be independently native (i.e. naturally-occurring) or modified (e.g. analogs or fragments of native HBV antigens).

In addition, they may originate from distinct HBV, especially from distict genotypes. Such a configuration permits to provide protection against a broader range of HBV genotypes or adaptation to a specific geographic region by using HBV genotype(s) that is/are endemic in this region or to a specific population of patients. Preferably, the one or more HBV antigens for use herein all originate from a genotype D HBV virus, with a specific preference for HBV isolate Y07587 (Genbank accession number Y07587 and Stoll-Becker et al, 1997, J. Virol. 71: 5399).

Core Antigen

In the native context, the HBV genome encodes a 183 amino acid-long core protein (or HBc), constitutive of the nucleocapsid. The C-terminus of the core protein is very basic and contains 4 Arg-rich domains which are predicted to bind nucleic acids as well as numerous phosphorylation sites.

As used herein, the term “core polypeptide”, “core antigen”, “HbcAg” or “HBc” refers to a polypeptide that retains at least 100 amino acid residues (preferably consecutive) of a native HBV core. Preferably, the encoded core antigen does not include any portion of a precore N-terminal extension. The present invention encompasses modified HBc or fragment thereof provided that the resulting core fragment or analog retains a significant immunogenic activity (preferably in the same range as or higher than the native counterpart). Exemplary HBc antigens for use herein are described in WO2013/007772. Among those described in this document, C-term truncation of at least 10 amino acid residues and at most 41 amino acid residues are preferred, and more particularly C-term truncation extending from the last core residue (position 183) to residue 143, 144, 145, 146, 147, 148 or 149 of the native core.

According to a preferred embodiment, the core antigen encoded by the combination of the present invention comprises (alternatively essentially consists of) an amino acid sequence that is at least 80% identical to SEQ ID NO: 1 or SEQ ID NO: 2.

Polymerase Antigen

In the native context, the HBV polymerase is about 832-845 amino acid residues long according to the HBV genotype and it is encoded in a long open reading frame (“P”) that overlaps the 3′end of the core gene and all the surface protein genes. The viral polymerase is a multifunctional protein composed of four domains, including three functional domains, respectively the terminal protein, polymerase and RNase H domains that catalyse the major steps in HBV replication (priming, DNA synthesis and removal of RNA templates). A non-essential spacer domain is present between the terminal protein and polymerase domains (see for example Radziwill et al., 1990, J. Virol. 64: 613-20; Bartenschlager et al., 1990, J. Virol. 64: 5324-32).

The catalytic sites responsible for enzymatic activities have been characterized. In particular, four residues forming the conserved YMDD motif (residues 538 to 541 numbered with respect to the native 832 residue long polymerase) have been shown essential to the DNA- and RNA-dependent DNA polymerase activity. RNase H activity has been mapped within the C-terminal portion (e.g. from position 680 to the C-terminus) and is based on a DEDD motif involving four non-consecutive amino acid residues, respectively Asp (D) in position 689, Glu (E) in position 718, Asp (D) in position 737 and Asp (D) in position 777 as well as few other amino acid residues including Val (V) in position 769 and Thr (T) in position 776. For purpose of illustration, the amino acid residues for HBV polymerase described herein are numbered by reference to a 832 amino acids long polymerase. It is within the reach of the skilled person to adapt the numeration of the amino acid residues to other polymerases (e.g. 843 or 845 amino acid long).

As used herein, the term “polymerase” refers to a polypeptide that retains at least 500 amino acid residues comprised in a native HBV pol antigen. Desirably, such at least 500 amino acid residues are spread over the three functional domains and preferably over the four domains normally present in a native HBV polymerase. Exemplary pol antigens for use herein are described in WO2013/007772.

A preferred embodiment relates to a polymerase antigen that is defective for the polymerase enzymatic activity exhibited by the native pol and comprising the deletion of at least 4 amino acid residues and at most 30 amino acid residues including the YMDD motif (positions 538-541 of a native polymerase of 832 amino acids). A more preferred pol antigen comprises the deletion of 7-30 amino acid residues including the YMDD motif as well as the neighboring VVL (positions 538-544) that, if present, may contribute to the formation of “junctional” epitopes (e.g. colinearly synthesized new epitopes) which are at risk of reducing or silencing the host's anti-polymerase immune response. The disruption of the polymerase activity exhibited by the resulting polymerase polypeptide can be evaluated using assays well known in the art (e.g. the endogenous polymerase assays described in Radziwill et al., 1990, J Virol. 64: 613-20).

Another preferred embodiment relates to modification(s) that functionally disrupt the RNaseH activity normally exhibited by a native HBV polymerase and the present invention encompasses the mutation(s) of any residues involved in native RNase H activity as described in WO2013/007772. Disruption of the RNase H activity can be evaluated using assays well known in the art (e.g. in vitro RNaseH activity assays or DNA-RNA tandem molecule analysis described in Radziwill et al., 1990, J Virol. 64: 613-20 or in Lee et al., 1997, Biochem. Bioph. Res. Commun. 233(2):401). Particularly preferred modification(s) are selected from the group consisting of a deletion extending from approximately position 710 to approximately 742, the substitution of the Asp residue in position 689 with a His (H) residue (D689H), the substitution of the Val residue in position 769 with a Tyr (Y) residue (V769Y), the substitution of the Thr residue in position 776 with a Tyr (Y) residue (T776Y), and the substitution of the Asp residue in position 777 with a His (H) residue (D777H), and any combination thereof. A particularly preferred polymerase antigen comprises (alternatively essentially consists of) an amino acid sequence which exhibits at least 80% of identity with the amino acid sequence shown in any of SEQ ID NO: 3-5.

HBs Antigen

In the native context, the HBV S ORF encodes three surface proteins all of which have the same C terminus but differ at their N-termini due to the presence of three in-frame ATG start codons that divide the S ORF into three regions, S (226 amino acids), pre-S2 (55 amino acids) and pre-S1 (108 amino acids), respectively. The large-surface antigen protein (L) is produced following translation initiation at the first ATG start codon and comprises 389 amino acid residues (preS1-preS2-S). The middle surface antigen protein (M) results from translation of the S region and the pre-S2 region starting at the second start ATG whereas the small surface antigen protein of 226 amino acids (S, also designated HBsAg) results from translation of the S region initiated at the third start ATG codon. The HBV surface proteins are glycoproteins with carbohydrate side chains (glycans) attached by N-glycosidic linkages.

In a preferred embodiment, the immunotherapeutic product comprised in the combination of the present invention encodes one or more HBs immunogenic domain(s) having from approximately 15 to approximately 100 amino acid residues, and preferably at least 20 and at most 60 consecutive amino acids of a native HBsAg protein. Preferably, the one or more HBsAg immunogenic domains used in the invention do not include any portions of HBV preS1 and preS2 polypeptides.

A vast choice of HBs immunogenic domains are described in the art (e.g. WO93/03764; WO94/19011; WO2011/015656; Desombere et al., 2000, Clin. Exp. Immunol 122: 390; Loirat et al., 2000, J. Immunol. 165: 4748; Schirmbeck et al., 2002, J. Immunol 168: 6253; and Depla et al., 2008, J. Virol. 82: 435). Particularly preferred are the env1 and env2 domains described in WO2013/007772. As a general guidance, “env1” and “env2” correspond to the portions of a native HBsAg (e.g. HBsAg of Y07587 isolate) from approximately position 14 to approximately position 51 and from approximately position 165 to approximately position 194, respectively and comprise an amino acid sequence which exhibits at least 80% of identity, with the amino acid sequence shown in SEQ ID NO: 6 or SEQ ID NO: 7.

Fusion Arrangement of HBV Antigens

In one embodiment, the immunotherapeutic product composition comprised in the combination of the invention encodes fusion in frame of HBV antigens. Fusions of particular interest comprise (i) a core antigen; (ii) a polymerase antigen and (iii) one or more HBsAg immunogenic domain(s) with a specific preference for a fusion of core and pol antigens comprising the HBsAg immunogenic domains inserted within the pol antigen in place of deleted portion(s) encompassing all or part of polymerase and/or RNaseH catalytic sites (see WO2013/007772). More preferred is a fusion protein comprising at its N-terminus, a C-term truncated core (e.g. positions 1 to 148 of a native HBc with the initiator Met) fused to the pol antigen (without initiator Met) having env1 domain inserted within the internal deletion aimed at disrupting polymerase activity and env2 within the internal deletion aimed at disrupting RNaseH activity (and if needed a STOP codon).

In a preferred aspect of this embodiment, the immunotherapeutic product encodes a HBV antigen fusion protein comprising (alternatively essentially consisting of) an amino acid sequence which exhibits at least 80% of identity with any of the amino acid sequence shown in SEQ ID NO: 8.

Polypeptide-Encoding Nucleic Acid Molecule and Generation of Vectorised Immunotherapeutic Product

The nucleic acid molecule encoding the one or more polypeptide(s) of interest (e.g. HBV antigens and fusion thereof) can be generated by a number of ways known to those skilled in the art (e.g. cloning, PCR amplification, DNA shuffling). For example, the polypeptide-encoding nucleic acid molecule can be isolated from any available source (e.g. biologic materials described in the art such as cDNA, genomic libraries, viral genomes or any prior art vector known to include it) using sequence data available to the skilled person and the sequence information provided herein, and then suitably inserted in the vectorised immunotherapeutic product by conventional molecular biology techniques. Alternatively, the polypeptide-encoding nucleic acid molecule can also be generated by chemical synthesis in automatized process (e.g. assembled from overlapping synthetic oligonucleotides or synthetic gene). Modification(s) can be generated by a number of ways known to those skilled in the art, such as chemical synthesis, site-directed mutagenesis, PCR mutagenesis, etc. For example, the nucleic acid molecules encoding the HBV antigen(s) may be isolated independently from an appropriate source of HBV or biologic materials described in the art (e.g. from HBV-containing cells, cDNA and genomic libraries, viral genomes or any prior art vector known to include it).

In particular, it might be advantageous to optimize the nucleic acid sequence for providing high level expression in a particular host cell or subject. It has been indeed observed that, the codon usage patterns of organisms are highly non-random and the use of codons may be markedly different between different hosts. As the polypeptide of interest may be from prokaryote (e.g. bacterial or viral antigen) origin, its coding sequence may have an inappropriate codon usage pattern for efficient expression in higher eukaryotic cells (e.g. human). Typically, codon optimization is performed by replacing one or more “native” codon corresponding to a codon infrequently used by one or more codon encoding the same amino acid which is more frequently used in the subject to treat. It is not necessary to replace all native codons corresponding to infrequently used codons since increased expression can be achieved even with partial replacement.

Further to optimization of the codon usage, expression can also be improved through additional modifications of the nucleotide sequence. For example, the nucleic acid sequence can be modified so as to prevent clustering of rare, non-optimal codons being present in concentrated areas and/or to suppress or modify “negative” sequence elements which are expected to negatively influence expression levels. Such negative sequence elements include without limitation AT-rich or GC-rich sequence stretches; unstable direct or inverted repeat sequences; and/or internal cryptic regulatory elements such as internal TATA-boxes, chi-sites, ribosome entry sites, and/or splicing donor/acceptor sites.

Moreover, it is advisable to degenerate the portions of nucleic acid sequences that show a high degree of sequence identity (e.g. the same antigen obtained from various serotypes of a given pathogen) so as to avoid homologous recombination problems during production process. The skilled person is capable of identifying such portions by sequence alignment. For example, the nucleotide sequences encoding HBs, HBc and pol antigens may be degenerated or truncated especially in the overlapping sequences to increase stability of the HBV targeted immunotherapeutic product or one of the common portion can be deleted (e.g. C-terminal portion of HBc overlapping N-terminal portion of pol). A preferred aspect of this embodiment comprises a nucleic acid molecule comprising (alternatively essentially consisting of) a nucleotide sequence which exhibits at least 80% of identity with any of the nucleotide sequence shown in SEQ ID NO: 9.

For the purposes of the present invention, the nucleic acid molecule(s) encoding the polypeptide(s) of interest can be inserted or included in the immunotherapeutic product according to the conventional practice in the art. Typically, with regard to viral vectors, the nucleic acid molecule(s) of interest is/are preferably inserted within a viral gene, an intergenic region, in a non-essential gene or in place of viral sequences.

The nucleic acid molecule(s) of interest is/are preferably inserted within the poxviral genome in a non-essential locus. Thymidine kinase gene is particularly appropriate for insertion in Copenhagen vaccinia vectors (see for example WO2010/130753; WO03/008533; U.S. Pat. Nos. 6,998,252; 5,972,597 and 6,440,422) and deletion II or III for insertion in MVA vector (WO97/02355; Meyer et al., 1991, J. Gen. Virol. 72: 1031-8). The general conditions for constructing and producing recombinant measles viruses are well known in the art. Insertion of the nucleic acid molecule(s) of interest between P and M genes or between H and L genes is particularly appropriate. For adenovirus-based immunotherapeutic product, E1 region is the preferred site of insertion and the nucleic acid molecule(s) to be expressed can be positioned in sense or antisense orientation relative to the natural transcriptional direction (see e.g. Chartier et al., 1996, J. Virol. 70: 4805-10 and WO96/17070). A preferred immunotherapeutic product comprises the nucleic acid molecule(s) encoding HBV antigen(s) (e.g. the fusion of SEQ ID NO: 8) cloned in the Ad5 genome in place of the deleted E1 region and in sense orientation.

In a specific embodiment, the one or more polypeptide(s) of interest are encoded in one or more vector(s) in the same or independent site of insertion, resulting in a single or multi vector composition.

Particularly preferred immunotherapeutic products for use herein are selected from the group consisting of:
A MVA virus encoding the MUC-1 antigen and human IL-2 as represented by TG4010 described in WO92/07000, U.S. Pat. No. 5,861,381 and Limacher and Quoix (2012, Oncolmmunology 1(5): 791-2);
A MVA virus encoding membrane anchored HPV-16 non-oncogenic E6 and E7 antigens and human IL-2 as represented by TG4001 described in WO99/03885;
A MVA virus encoding the FCU1 gene as represented by TG4023 (WO99/54481);
A MVA virus encoding one or more Mtb antigens (see e.g. WO2014/009438 and WO2015/104380); and
An Ad (Ad5) virus encoding a fusion of HBc, pol, env1 and env2 as represented by TG1050 (also named AdTG18201 as described in WO2013/007772).

Expression of the Nucleic Acid Molecule Encoding the Polypeptide(s) of Interest

Expression of an antigen or other protein in the immunotherapeutic product for use in the present invention is accomplished using techniques known to those skilled in the art. Briefly, the encoding nucleic acid molecule(s) is/are inserted into the plasmid or viral vector in such a manner to be operably linked to suitable regulatory elements for expression in the desired host cell or subject. It will be appreciated by those skilled in the art that the choice of the regulatory sequences can depend on such factors as the gene itself, the virus into which it is inserted, the host cell or subject, the level of expression desired, etc.

As used herein, the term “regulatory elements” or “regulatory sequence” refers to any element that allows, contributes or modulates the expression of the nucleic acid molecule(s) in a given host cell or subject, including replication, duplication, transcription, splicing, translation, stability and/or transport of the nucleic acid(s) or its derivative (i.e. m RNA). As used herein, “operably linked” means that the elements being linked are arranged so that they function in concert for their intended purposes. For example, a promoter is operably linked to a nucleic acid molecule if the promoter effects transcription from the transcription initiation to the terminator of said nucleic acid molecule in a permissive host cell.

Suitable promoters for use herein can be constitutive directing expression of the nucleic acid molecule(s) in many types of cells or specific to certain types of cells or tissues or regulated in response to specific events or exogenous factors (e.g. by temperature, nutrient additive, hormone, etc.) or according to the phase of a viral cycle (e.g. late or early). One may also use promoters that are repressed during the production step in response to specific events or exogenous factors, in order to optimize production of the immunotherapeutic product and circumvent potential toxicity of the expressed polypeptide(s).

Exemplary constitutive promoters for expression in recombinant viral and plasmid vectors include, but are not limited to, the cytomegalovirus (CMV) immediate early promoter (U.S. Pat. No. 5,168,062), the RSV promoter, the adenovirus major late promoter, the phosphoglycero kinase (PGK) promoter (Adra et al., 1987, Gene 60: 65-74), the thymidine kinase (TK) promoter of herpes simplex virus (HSV)-1 and the T7 polymerase promoter (WO98/10088). Vaccinia virus promoters are particularly adapted for expression in recombinant poxviruses. Representative examples include without limitation the vaccinia 7.5K, HSR, 11K7.5 (Erbs et al., 2008, Cancer Gene Ther. 15(1): 18-28), TK, pB2R, p28, p11 and K1L promoter, synthetic promoters such as those described in Chakrabarti et al. (1997, Biotechniques 23: 1094-7; Hammond et al, 1997, J. Virol Methods 66: 135-8; and Kumar and Boyle, 1990, Virology 179: 151-8) as well as early/late chimeric promoters. Promoters suitable for measles viruses include without limitation any promoter directing expression of measles transcription units (Brandler and Tangy, 2008, CIMID 31: 271).

Those skilled in the art will appreciate that the regulatory elements controlling the expression of the nucleic acid molecule(s) of interest may further comprise additional elements for proper initiation, regulation and/or termination of transcription (e.g. polyA transcription termination sequences), mRNA transport (e.g. nuclear localization signal sequences), processing (e.g. splicing signals), stability (e.g. introns and non-coding 5′ and 3′ sequences), translation (e.g. an initiator Met, STOP codon, tripartite leader sequences, IRES ribosome binding sites, signal peptides, etc.) and purification steps (e.g. a tag).

Production of Vector-Based Immunotherapeutic Product

Effective conditions for the production of the immunotherapeutic product for use herein include a) culturing a producer (e.g. permissive) host cell, b) transfecting or infecting the cultured producer host cell, c) culturing the transfected or infected host cell under suitable conditions so as to allow the production of the product (e.g. infectious viral particles), d) recovering the produced immunotherapeutic product from the culture of said cell and optionally e) purifying said recovered immunotherapeutic product.

In step a), producer cells are chosen depending on the type of vector to be prepared. Replication-defective recombinant adenoviruses are typically propagated and produced in a cell that supplies in trans the adenoviral protein(s) encoded by those genes that have been deleted or inactivated, thus allowing the virus to replicate in the cell. Suitable cell lines for complementing E1-deleted adenoviruses include the HEK-293 (Graham et al., 1997, J. Gen. Virol. 36: 59-72), HER-96, PER-C6 (e.g. Fallaux et al., 1998, Human Gene Ther. 9: 1909-1917; WO97/00326), 293-ORF6 cells (described in, e.g., WO 95/34671 and Brough et al., 1997, J. Virol., 71: 9206-13) and any derivative of these cell lines. But any other cell line described in the art can also be used in the context of the present invention, especially cell lines approved for producing products for human use.

MVA is strictly host-restricted and is typically amplified on avian cells, either primary avian cells (such as chicken embryo fibroblasts (CEF) prepared from chicken embryos obtained from fertilized eggs) or immortalized avian cell lines. Representative examples of suitable avian cell lines for MVA production include without limitation the Cairina moschata cell lines immortalized with a duck TERT gene (see e.g. WO2007/077256, WO2009/004016, WO2010/130756 and WO2012/001075); avian cell line immortalized with a combination of viral and/or cellular genes (see e.g. WO2005/042728); a spontaneously immortalized cell (e.g. the chicken DF1 cell line disclosed in U.S. Pat. No. 5,879,924); or immortalized cells which derive from embryonic cells by progressive severance from growth factors and feeder layer (e.g. Ebx chicken cell lines disclosed in WO2005/007840 and WO2008/129058).

For other vaccinia virus or other poxvirus strains, in addition to avian primary cells (such as CEF) and avian cell lines, many other non-avian cell lines are available for production, including human cell lines such as HeLa (ATCC-CRM-CCL-2™ or ATCC-CCL-2.2™), MRC-5, HEK-293; hamster cell lines such as BHK-21 (ATCC CCL-10), and Vero cells. In a preferred embodiment, non-MVA vaccinia virus are amplified in HeLa cells (see e.g. WO2010/130753).

Producer cells are preferably cultivated in a medium free of animal- or human-derived products, using a chemically defined medium with no product of animal or human origin. Culturing is carried out at a temperature, pH and oxygen content appropriate for the producer cell. Such culturing conditions are within the expertise of one of ordinary skill in the art. In particular, while growth factors may be present, they are preferably recombinantly produced and not purified from animal material. Suitable animal-free medium media are commercially available, for example VP-SFM medium (Invitrogen) for culturing CEF producer cells. Producer cells are preferably cultivated at a temperature comprised between +30° C. and +38° C. (more preferably at about +37° C.) for between 1 and 8 days (preferably for 1 to 5 days for CEF and 2 to 7 days for immortalized cells) before infection. If needed, several passages of 1 to 8 days may be made in order to increase the total number of cells.

In step b), producer cells are infected/transfected by the immunotherapeutic product. Infection of producer cells by the virus-based immunotherapeutic product is conducted using an appropriate multiplicity of infection (MOI) to permit productive infection which can be as low as 0.001 (more preferably between 0.05 and 5) for VV vector (e.g. MVA) and between 0.05 and 200 (typically between 0.1 and 50) for Ad-based immunotherapeutic product.

In step c), infected producer cells are then cultured under appropriate conditions well known to those skilled in the art until progeny viral vector (e.g. infectious virus particles) is produced. Culture of infected producer cells is also preferably performed in a chemically defined medium (which may be the same as or different from the medium used for culture of producer cells and/or for infection step) free of animal- or human-derived products at a temperature between +30° C. and +37° C., for 1 to 5 days.

In step d), the viral vector produced in step c) is collected from the culture supernatant and/or the producer cells. Recovery from producer cells (and optionally also from culture supernatant), may require a step allowing the disruption of the producer cell membrane to allow the liberation of the vector from producer cells. The disruption of the producer cell membrane can be induced by various techniques well known to those skilled in the art, including but not limited to: freeze/thaw, hypotonic lysis, sonication, microfluidization, or high-speed homogenization.

Viral vectors may then be further purified, using purification steps well known in the art. Various purification steps can be envisaged, including clarification, enzymatic treatment (e.g. endonuclease, protease, etc), ultracentrifugation (e.g. cesium chloride gradient), chromatography and/or filtration steps. Appropriate methods are described in the art (e.g. WO2007/147528; WO2008/138533, WO2009/100521, WO2010/130753, WO2013/022764, WO96/27677, WO98/00524, WO98/22588, WO98/26048, WO00/40702, EP1016711 and WO00/50573).

Immunotherapeutic Product Composition

In one embodiment, the immunotherapeutic product composition comprises a pharmaceutically acceptable vehicle. The term “pharmaceutically acceptable vehicle” is intended to include any and all carriers, solvents, diluents, excipients, adjuvants, dispersion media, coatings, antibacterial and antifungal agents, absorption agents and the like compatible for human use.

Various formulations can be envisaged in the context of the invention, either liquid or freeze-dried form to ensure stability under the conditions of manufacture and long-term storage (i.e. for at least 6 months) at freezing (e.g. −70° C., −20° C.), refrigerated (e.g. 4° C.) or ambient (e.g. 20-25° C.) temperature.

Liquid compositions generally include a liquid vehicle such as physiological saline solution, Ringer's solution, Hank's solution, saccharide solution (e.g. glucose, trehalose, saccharose, dextrose, etc) and other aqueous physiologically balanced salt solutions (see for example the most current edition of Remington: The Science and Practice of Pharmacy, A. Gennaro, Lippincott, Williams & Wilkins). Non-aqueous vehicles, such as fixed oils, sesame oil, ethyl oleate, or triglycerides may also be used. Other useful formulations include suspensions containing viscosity-enhancing agents, such as sodium carboxymethylcellulose, sorbitol, glycerol or dextran. Excipients can also contain minor amounts of additives, such as substances that enhance isotonicity and chemical stability (e.g. human serum albumin).

It might also be beneficial to also include a monovalent salt so as to ensure an appropriate osmotic pressure. Said monovalent salt may notably be selected from NaCl and KCl, preferably in a concentration of 10 to 500 mM.

Freeze dried (lyophilized) immunotherapeutic product composition may also include a cryoprotectant so as to protect the immunotherapeutic product at low storage temperature. Representative examples of cryoprotectants suitable for use in the context of the present invention are sucrose (or saccharose), trehalose, maltose, lactose, mannitol, sorbitol and glycerol, preferably in a concentration of 0.5 to 20% (weight in g/volume in L, referred to as w/v). For example, sucrose may be present in a concentration of 5 to 15% (w/v), with a specific preference for about 10%. The presence of high molecular weight polymers such as dextran or polyvinylpyrrolidone (PVP) is particularly suited to protect the biological product during the vacuum drying and freeze-drying steps (see e.g. WO03/053463; WO2006/0850082; WO2007/056847; WO2008/114021) and to assist in the formation of the cake during freeze-drying (see EP1418942 and WO2014/053571).

Whatever the formulation (liquid, frozen or lyophilized), the immunotherapeutic product composition is preferably buffered at physiological or slightly basic pH (e.g. from approximately pH 7 to approximately pH 9 with a specific preference for a pH comprised between 7 and 8 and more particularly close to 7.5). Suitable buffers include without limitation TRIS (tris(hydroxymethyl)methylamine), TRIS-HCl (tris(hydroxymethyl)methylamine-HCl), HEPES (4-2-hydroxyethyl-1-piperazineethanesulfonic acid), phosphate buffer (e.g. PBS), bicarbonate buffer (comprising a mixture of Na₂HPO₄ and KH₂PO₄ or a mixture of Na₂HPO₄ and NaH₂PO₄), ACES (N-(2-Acetamido)-aminoethanesulfonic acid), PIPES (Piperazine-N,N′-bis(2-ethanesulfonic acid)), MOPSO (3-(N-Morpholino)-2-hydroxypropanesulfonic acid), MOPS (3-(N-morpholino)propanesulfonic acid), TES (2-{[tris(hydroxymethyl)methyl]amino}ethanesulfonic acid), DIPSO (3-[bis(2-hydroxyethyl)amino]-2-hydroxypropane-1-sulfonic acid), MOBS (4-(N-morpholino)butanesulfonic acid), TAPSO (3-[N-Tris(hydroxymethyl)methylamino]-2-hydroxypropanesulfonic Acid), HEPPSO (4-(2-Hydroxyethyl)-piperazine-1-(2-hydroxy)-propanesulfonic acid), POPSO (2-hydroxy-3-[4-(2-hydroxy-3-sulfopropyl)piperazin-1-yl]propane-1-sulfonic acid), TEA (triethanolamine), EPPS (N-(2-Hydroxyethyl)-piperazine-N′-3-propanesulfonic acid), and TRICINE (N-[Tris(hydroxymethyl)-methyl]-glycine). TRIS-HCl, TRIS, Tricine, HEPES and phosphate buffer are preferred in the context of the invention. For illustrative purposes, a buffer concentration of 5 to 50 mM is appropriate.

The immunotherapeutic composition (especially liquid composition) may further comprise a pharmaceutically acceptable chelating agent, and in particular an agent chelating dications for improving stability, with a specific preference for ethylenediaminetetraacetic acid (EDTA). The pharmaceutically acceptable chelating agent is preferably present in a concentration of at least 50 μM (e.g. 50 to 1000 μM) with a specific preference for a concentration close to 150 μM.

Additional compounds may further be present to increase stability of the formulated immunotherapeutic product composition. Such additional compounds include, without limitation, C₂-C₃ alcohol (desirably in a concentration of 0.05 to 5% (volume/volume or v/v)), sodium glutamate (desirably in a concentration lower than 10 mM), non-ionic surfactant (Evans et al. 2004, J Pharm Sci. 93: 2458-75, Shi et al., 2005, J Pharm Sci. 94:1538-51, U.S. Pat. No. 7,456,009, US2007/0161085) such as Tween 80 (also known as polysorbate 80) at low concentration below 0.1%. Divalent salts such as MgCl₂ or CaCl₂ have been found to induce stabilization of various biological products in the liquid state (see Evans et al. 2004, J Pharm Sci. 93:2458-75 and U.S. Pat. No. 7,456,009). Amino acids, and in particular histidine, arginine or methionine, have been found to induce stabilization of various viruses in the liquid state (see Evans et al., 2004, J Pharm Sci. 93:2458-75, U.S. Pat. No. 7,456,009, US2007/0161085, U.S. Pat. No. 7,914,979, WO2014/029702 and WO2014/053571).

In a further embodiment, the immunotherapeutic product composition may be adjuvanted to further enhance immunity. Representative examples of suitable adjuvants include, without limitation, alum, mineral oil emulsion such as, Freunds complete and incomplete (IFA), lipopolysaccharides (Ribi et al., 1986, Immunology and Immunopharmacology of Bacterial Endotoxins, Plenum Publ. Corp., NY, p 407-419), saponins such as ISCOMATRIX, AbISCO, Q521 (Sumino et al., 1998, J. Virol. 72: 4931; WO98/56415), imidazo-quinoline compounds such as Imiquimod (Suader, 2000, J. Am Acad Dermatol. 43:S6), S-27609 (Smorlesi, 2005, Gene Ther. 12: 1324) and related compounds such as those described in WO2007/147529; cationic peptides such as IC-31 (Kritsch et al., 2005, J. Chromatogr Anal. Technol. Biomed. Life Sci. 822: 263-70), polysaccharides such as Adjuvax and squalenes and oil in water emulsions such as MF59, double-stranded RNA analogs such as poly(I:C), single-stranded oligodeoxynucleotides such as CpG, bacterial proteins such as flagellin, chitosan or derivates thereof, polyphosphazenes.

The formulation of the immunotherapeutic product composition can also be adapted to the mode of administration to ensure proper distribution and release in vivo. For example, gastro-resistant capsules and granules are particularly appropriate for oral administration, suppositories for rectal or vaginal administration, optionally in combination with absorption enhancers useful to increase the pore size of the mucosal membranes. Such absorption enhancers are typically substances having structural similarities to the phospholipid domains of the mucosal membranes (such as sodium deoxycholate, sodium glycocholate, dimethyl-beta-cyclodextrin, lauryl-1-lysophosphatidylcholine). Another and particularly appropriate example is a formulation adapted to the administration through microneedle means (e.g. transcutaneous or intradermal patches). Such a formulation may comprise resuspension of the immunotherapeutic product in endotoxin-free phosphate-buffered saline (PBS).

MDSC Modulator(s)

In a further aspect, the present invention provides one or more MDSC modulator(s) for use for treating a pathological condition (e.g. a proliferative disease or a chronic infectious disease) in a subject in need therefore in combination with at least a composition comprising an immunotherapeutic product as described herein.

As mentioned before, the one or more MDSC modulator(s) in use herein may independently act at any step of the MDSC's signaling pathway. Suitable MDSC modulators useful in effecting the methods of the present invention include, without limitation, small molecules (chemical or synthetic), proteins, peptides (e.g. soluble receptors), amino acids or derivates thereof, antibodies, nucleic acid molecules, etc. Particularly preferred in the context of the invention, are Vitamin A, D3 or E derivatives such as ATRA, chemotherapy drugs (e.g. gemcitabine, 5-fluorouracile, etc.) and inhibitors of phosphodiesteraseenzyme(s) (PDE).

Preferred MDSC modulators in the context of the present invention are capable of antagonizing at least partially (e.g. a minimum of 20% reduction) the activity of one or several subtypes of PDE and, in particular, of phosphodiesterase subtype 5 (PDE-5). Such PDE inhibitors may be non-selective (designated herein as “PDE inhibitor” for inhibiting various PDE subtypes) as well as selective towards PDE-5 (designated herein as “PDE5 inhibitor”. Representative examples of non-selective PDE inhibitors include, but are not limited to caffeine, 1,3-dimethyl xanthine (theophylline) and 3-isobutyl-1-methylxanthine (IBMX).

PDE5 Inhibitors and Sildenafil

Generally speaking, PDE5 is responsible for degradation of cyclic guanosine monophosphate (cGMP) and, thus, controls cGMP levels. The production of cGMP requires the presence of soluble guanylyl cyclase (sGC) bound to nitric oxide (NO) via a heme on its beta subunit. The catalytic activity of PDE5 is therefore dependent on levels of sCG and by extension NO (Corbin et al., 2000, Eur J Biochem 267(9): 2760-7).

The term “PDE-5 inhibitor” is used herein to include any compound that partially or fully blocks, inhibits, reduces, or neutralizes the activity of a PDE-5 enzyme (e.g. a human PDE-5), in particular its ability to hydrolyze cGMP to the inactive GMP.

In the context of the present invention, the PDE5 inhibitor can exert its antagonist effect through various pathways, e.g. by occupying the enzyme's active site, by competing or interacting with any of its ligand(s), etc., in particular with cGMP. The term “PDE 5 inhibitor” encompass the inhibitor per se, pharmaceutical acceptable salts thereof (e.g. citrate, mesylate, maleate, etc), enantiomer, racemic mixture thereof, solvate and composition thereof as well as analogs thereof. As used herein, the term “analog” refers to a chemical compound that is structurally similar to another compound but differs slightly in composition (as in the replacement of one atom by an atom of a different element or in the presence of a particular functional group, or the replacement of one functional group by another functional group). Thus, an analog is a compound that is similar or comparable in function and appearance, but has a different structure or origin with respect to the reference compound.

Techniques for preparing or separating racemic PDE 5 inhibitors are known (see, for example, Gao, et al, 2007, J. Chromatogr. Sci., 45:540-543). The suitability of any particular PDE5 inhibitor can be readily evaluated as described in the literature (Tinsley et al., 2010, Cancer Prevention Res 3(10): 1303-13), e.g., using IMAP fluorescence polarization PDE assay (Molecular Devices) or [3H]cGMP scintillation proximity enzyme assay kits (Amersham). Evaluation of its toxicity, absorption, metabolism, pharmacokinetics, etc., may be performed in accordance with standard pharmaceutical practice. Desirably, the PDE5 inhibitors in use in the present invention is a compound displaying desirable selectivity for PDE5 such as pyrazolopyrimidinones. Selectivity for PDE5 may be variable, e.g. with IC₅₀ ranging from 0.01 nM to 50 nM and preferably from 0.1 to 5 nM.

Non-limiting examples of PDE5 inhibitors include, but are not limited to, avanafil, lodenafil, mirodenafil, sildenafil, (or analogs thereof, for example, actetildenafil, hydroxyacetildenafil, dimethylsildenafil or thiomethisosildenafil), tadalafil, vardenafil, udenafil, zaprinast, icariin, sulfoaildenafil and benzamidenafil with a specific preference for sildenafil, vardenafil and tadalafil. The structures of these compounds are well known in the art and many PDE5 inhibitors are commercially available.

The term “sildenafil” as used herein includes the free base form of this compound (chemical name as 1-[[3-(4,7-dihydro-1-methyl-7-oxo-3-propyl-1H-pyrazolo[4,3-d] pyrimidin-5-yl)-4-ethoxyphenyl] sulfonyl]-4-methylpiperazine; having a molecular weight of 474.6) as well as pharmacologically acceptable acid addition salts thereof formed with organo-carboxylic acids, organo-sulphonic acids or inorganic acids. Therefore, reference to sildenafil also includes sildenafil salts such as sildenafil citrate (Molecular Weight: 666.7) and sildenafil mesylate. Sildenafil was originally disclosed in U.S. Pat. Nos. 5,250,534, 6,469,012 and EP 463 756. Methods for the preparation of sildenafil are disclosed in number of documents (see for example EP 812 845; U.S. Pat. No. 6,204,383; WO01/019827; WO2005/067936; WO2008/074512; Bioorg. Med. Chem. Lett. 2000, 10, 1983-1986). Sildenafil has originally been approved by regulatory authorities for the treatment of cardiovascular diseases, such as angina, hypertension, heart failure, atherosclerosis, etc. Later it was found that this compound is particularly effective in the treatment of male erectile dysfunction disease (WO94/28902). Sildenafil citrate is commonly marketed as VIAGRA® (for treatment of erectile dysfunction) and REVATIO® (for treatment of pulmonary hypertension), both manufactured by Pfizer Pharmaceuticals. Generic versions of sildenafil citrate are also available. VIAGRA is commonly supplied as 25, 50 or 100 mg tablets whereas REVATIO® is most often supplied as 20 mg tablets to be taken by oral route. REVATIO® is also available in injectable form containing 10 mg of sildenafil citrate per 12.5 ml of solution to be administered intravenously. But other sildenafil formulations are being currently developed and also suitable in the context of the invention, e.g. controlled released formulations for sublingual or buccal administrations (see for example WO00/54777); trans-mucosal formulations (see for example WO2000/075597), oral spray formulations (see for example EP 2575765), pulse-released formulations (see for example EP 2374460), nasal formulations (see for example WO99/66933) and transdermal formulations (see for example EP 2968130).

Tadalafil (chemical name: (6R-trans)-6-(1,3-benzodioxol-5-yl)-2,3,6,7,12,12a-hexahydro-2-methyl-pyrazino [1′, 2′:1,6] pyrido [3,4-b] indole-1,4-dione) is marketed by Lilly ICOS LLC (Indianapolis, Ind.) under the trade name Cialis® in 2.5 mg, 5 mg, 10 mg, and 20 mg pill form for treating erectile dysfunction in men and under the name Adcirca® (40-mg daily doses) for the treatment of pulmonary arterial hypertension. Tadalafil is also manufactured and sold under the name of Tadacip® by the Indian pharmaceutical company Cipla in doses of 10 mg and 20 mg.

Vardenafil (chemical name 4-[2-Ethoxy-5-(4-ethylpiperazin-1-yl)sulfonyl-phenyl]-9-methyl-7-propyl-3,5,6,8-tetrazabicyclo [4.3.0] nona-3,7,9-trien-2-one) is also used for treating erectile dysfunction and sold under the trade names Levitra® (Bayer Pharmaceuticals Corporation, GSK and Schering Plough), Staxyn® in India, and Vivanza® in Italy. Vardenafil's indications and contra-indications are the same as with other PDE5 inhibitors; it is closely related in function to sildenafil citrate (Viagra) and tadalafil (Cialis). The difference between the vardenafil molecule and sildenafil citrate is a nitrogen atom's position and the change of sildenafil's piperazine ring methyl group to an ethyl group.

The PDE-5 inhibitors in use in the invention may be administered either alone or in combination with one or more compounds acting on PDE5 such as Arginine (WO2012/019127) and/or nitric oxide (“NO”) donor drugs. For example, according to the manufacturer, in addition to the active ingredient, sildenafil citrate, each tablet contains the following inactive ingredients: microcrystalline cellulose, anhydrous dibasic calcium phosphate, croscarmellose sodium, magnesium stearate, hydroxypropyl methylcellulose, titanium dioxide, lactose, triacetin, and blue colouring agent.

Combination Therapy

“Combination therapy” and any variation such as “combined use” refers to the action of delivering to the same subject both an immunotherapeutic product composition and one or more MDSC modulator(s). Such a combination encompasses the cases where the individual entities are administered to the subject as a single composition (together in the same composition) or separately (i.e. dissociate arrangement), in which case the immunotherapeutic product composition and the MDSC modulator(s) may be administered concurrently, sequentially, in an interspersed manner or in any combination of these types of administration.

“Concurrently” means to administer each of the immunotherapeutic product composition and one or more MDSC modulator(s) essentially at the same time or over the same period of time (e.g., within one hour or less), although not necessarily in the same composition. “Sequentially” refers to “one after the other” meaning one entity being administered first followed by the administration of the second at a suitable period of time. In other words, The MDSC modulator(s) therapy can be conducted before initiating the immunotherapeutic product treatment or vice versa (the MDSC modulator is administered after the immunotherapeutic product composition). “Interspersed” means intermixed administrations of the immunotherapeutic product and MDSC modulator(s) at various time intervals.

In the context of the invention, the immunostimulatory combination of the present invention can be used for prophylaxis (e.g. to reduce the risk of having a given disease or pathological condition) and/or for therapy (e.g. in a subject diagnosed as having a given disease or pathological condition). When “prophylactic” use is concerned, the immunostimulatory combination is administered at a dose sufficient to prevent or to delay the onset and/or establishment and/or relapse of a pathologic condition, especially in a subject at risk. For “therapeutic” use, the immunostimulatory combination is administered at a dose sufficient to slow down, cure, improve or control the occurrence or the progression of the targeted disease or pathologic condition or alleviate one or more symptoms related to or associated with said disease or condition. Therapeutic use is preferred in the context of the present invention.

Doses

It is appreciated that optimal concentrations of each entity of the combination can be routinely determined by a practitioner in the light of the relevant circumstances (age, body weight, symptoms, clinical state, route of administration, duration of the treatment, etc.). Further refinement of the calculations can be necessary to adapt the appropriate dosage for a subject or a group of subjects.

As a general guidance, suitable individual dose for a virus-based immunotherapeutic product varies from approximately 10⁴ to approximately 10¹³ vp (viral particles), iu (infectious unit) or pfu (plaque-forming units) depending on the viral vector and quantitative technique used. More specifically, individual adenovirus doses from about 10⁵ to about 10¹³ vp are suitable, preferably from about 10⁶ vp to about 5×10¹² vp, more preferably from about 10⁷ vp to about 10¹² vp; doses of about 10⁸ vp to about 5×10¹¹ vp being particularly preferred, especially doses of about 10⁹ vp, about 10¹⁰ vp or about 10¹¹ vp. Individual doses which are suitable for vaccinia virus-based immunotherapeutic product comprise from about 10⁴ to about 10¹³ pfu. More specifically, suitable doses of replication-defective vaccinia-based composition such as MVA comprises from about 10⁴ to about 10¹² pfu, preferably from about 10⁵ pfu to about 10¹¹ pfu, more preferably from about 10⁶ pfu to about 10¹⁰ pfu; doses of about 10⁷ pfu to about 10⁹ pfu being particularly preferred especially for human use. Individual doses which are suitable for oncolytic Vaccinia-based immunotherapeutic product comprise from about 10⁵ to about 10¹³ pfu, preferably from about 10⁶ pfu to about 10¹¹ pfu, more preferably from about 10⁷ pfu to about 10¹⁰ pfu; doses of about 10⁸ pfu to about 5×10⁹ pfu being particularly preferred especially for human use. The quantity of virus present in a sample can be determined by routine titration techniques, e.g. by counting the number of plaques (pfu) following infection of permissive cells (e.g. 293 or PERC6 for Ad, BHK-21 or CEF for MVA, HeLa for VV), by measuring the A260 absorbance (vp titers), or still by quantitative immunofluorescence, e.g. using anti-virus antibodies (iu titers). Suitable dosage for a plasmid-based immunotherapeutic product varies from 10 μg to 50 mg, advantageously from 100 μg to 20 mg and preferably from 0.5 mg to 10 mg.

A suitable dose of MDSC modulator(s), including any of the PDE-5 inhibitors described herein or known in the art, will vary from modulator to modulator. In general, guidelines are provided by their manufacturers. Typically, the MDSC modulator dosage is an effective dose to decrease the MDSC's immunosuppressive activity in a subject, such that the activity of the immunotherapeutic product composition is improved as compared to in the absence of the MDSC modulator.

MDSC modulator(s) is typically administered at doses varying from about 0.1 mg to about 500 mg including any intermediate whole integer dosage in 5 mg increments (i.e., 0.5 mg; 1 mg, 1.5 mg, etc.). For illustrative purposes, suitable individual doses for PDE5 inhibitor(s) and particularly for sildenafil (including analogs thereof) vary from about from about 0.5 mg to about 250 mg, preferably from about 1 mg to about 200 mg, more preferably from about 1 mg to about 150 mg, and more specifically from about 1.5 mg to about 100 mg, taken in one or more doses of 2, 5, 10, 20, 25 mg, 50 mg or 100 mg. However, lower doses may be envisaged for local administration.

The MDSC modulator(s) (e.g. sildenafil) is preferably administered orally (as a tablet) at a periodocity of time which can be defined by a practicionner and the periodicity can vary over the course of treatment. For illustrative purposes, it can be every day, every 2 days, twice a week, weekly or bi-weekly. For example, a daily 60 mg dose may be taken at one time (e.g. 3 tablets of 20 mg) or in several subdoses (e.g. one 20 mg tablet subdose taken at each meal).

In a preferred embodiment, the immunotherapeutic product is an adenovirus and MDSC modulator is a PDE5 inhibitor and more particularly sildenafil.

A particularly preferred combination of the present invention comprises a) a composition comprising from about 10⁸ vp to about 5×10¹¹ vp of an adenovirus-based immunotherapeutic product, and b) from about 1 mg to about 150 mg of a PDE5 inhibitor. Even more preferred is an immunostimulatory combination comprising a) about 10⁹ vp (i.e. 8×10⁸ to 2×10⁹), about 10¹⁰ vp (i.e. 8×10⁹ to 2×10¹⁰) or about 10¹¹ vp (i.e. 8×10¹⁰ to 2×10¹¹) of an Ad vector encoding one or more HBV antigen(s), especially HBc, pol and HBs immunogenic domains (such as the fusion thereof represented by SEQ ID NO: 8) and b) daily or every 2 days dose from about 1.5 mg to about 100 mg of sildenafil, with a specific preference for daily doses of 2, 5, 10, 20, 25, 50 or 100 mg.

Administration

Administration of the immunotherapeutic product composition and the MDCS modulator(s) can be independently parenteral, mucosal and/or topical. Parenteral routes are intended for administration by injection or infusion and encompass systemic as well as local routes. Suitable routes of administration will be apparent to those of skill in the art, depending on the type of pathological condition to be prevented or treated and/or the combination itself, and/or the target tissue. Various acceptable routes of administration include, but are not limited to intravenous, intravascular, intra-arterial (into the corononary artery), intradermal, transcutaneous, subcutaneous, intramuscular, intraperitoneal, intraocular, intracranial, intraspinal, intraarticular, intranodal (e.g. into a lymph node), intrapleural and intratumoral (into a tumor or its close vicinity) routes as well as scarification. Mucosal administrations include without limitation, oral/alimentary, intranasal, intratracheal, nasopharyngeal, intrapulmonary, intravaginal, and intra-rectal routes. Intranasal delivery can include nose drops or intranasal injection, and intraocular delivery can include eye drops. Transcutaneous and intradermal delivery is also suitable in the context of the invention as well as inhalation (e.g., aerosol). Preferred routes of administration for the immunotherapeutic product composition include intradermal, transcutaneous, intravenous, intramuscular, subcutaneous and intratumoral whereas the one or more MDSC modulator(s) are preferably given by oral, sublingual or intravenous route. Subcutaneous administration of the immunotherapeutic product composition and oral administration of the MDSC modulator(s) are particularly preferred.

Administrations may use standard needles and syringes or any device available in the art capable of facilitating or improving delivery including for example catheters, electric syringe, Quadrafuse injection needles, needle-free injection devices (e.g. Biojector™ device), infusion pumps etc. Electroporation may also be implemented to facilitate intramuscular administration. Administration of the immunotherapeutic product can also be performed using transcutaneous or intradermal means (e.g. patch and the like). Systems are being developed using solid, hollow, coated or dissolvable microneedles (see e.g., Van der Maaden et al., 2012, J. Control release 161: 645-55) and preferred are silicon and sucrose microneedle patches (see, e.g., Carrey et al., 2014, Sci Rep 4: 6154 doi 10.1038; and Carrey et al., 2011, PLoS ONE, 6(7) e22442).

Time Course Administration

In accordance with the present invention, the immunotherapeutic product composition and the one or more MDSC modulator(s) may be administered one or several times, by the same or different routes, at the same or different sites, with the same or different dosages and the sequence of the multiple administrations and intervals in between may vary. The doses can vary for each administration within the range described above. Intervals between the various administrations (e.g. between the immunotherapeutic product administrations, between the MDSC modulator administrations and/or between the immunotherapeutic product and MDSC modulator administrations) can be regular or irregular. One may also proceed via sequential cycles of administrations that are repeated after a rest period.

In a preferred embodiment, a virus-based immunotherapeutic product composition is administered via 1 to 3 sequential cycles, each including 1 to 6 administrations, preferably with a time interval of 3 days to 2 weeks (preference for weekly intervals) between each administration within a cycle and a rest period of few weeks to few months between 2 cycles. For example, 1 to 6 weekly administrations of the immunotherapeutic product composition, optionally followed by one or more monthly administration(s) (e.g. 1, 2, 3, 4, 5, etc.) are appropriate.

Various protocols using the combination of the present invention are contemplated by the invention, and these examples should be considered to be non-limiting examples.

In one embodiment, the MDSC modulator therapy is administered more frequently than the immunotherapeutic product composition. For example, the immunotherapeutic product composition is administered over a period of 2 or 3 weeks (e.g. 3 weekly administrations) whereas MDSC modulator(s) therapy is preferably given for at least one-month period (e.g. daily or every 2 days).

In another embodiment, the immunotherapeutic product therapy and the MDSC modulator(s) therapy overlap at least partially. In one aspect of this embodiment, administrations of the immunotherapeutic product and MDSC modulator(s) start at approximately the same time period. In another and preferred aspect, the MDSC modulator(s) are given to the subject before initiating administration(s) of the immunotherapeutic product composition. More preferably, the MDSC modulator(s) therapy starts prior to immunotherapeutic product immunotherapy, with continuation of MDSC modulator therapy during immunotherapeutic product immunotherapy, and optionally, with continuation of MDSC modulator therapy for a period of time after immunotherapeutic product immunotherapy. For example, MDSC modulator(s) may be given to the subject at least one week (e.g. 1, 2, 3, 4 weeks or more) before initiating administrations of the immunotherapeutic product, so that MDSC modulator(s) could reduce immunosuppressive cell population and increasing immune T cells within inflammatory infiltrate before immunotherapeutic product could instigate a proper immune response. One exemplary regimen comprises oral administrations of the MDSC modulator(s) every day or every 2 days over a month period (J0 to J30) and 3 weekly subcutaneous administrations of the immunotherapeutic product initiated at least one week after the start of MDSC modulator(s) therapy (e.g. at J7, J14 and J21). In still another aspect, the administration of the immunotherapeutic product composition is initiated before starting the MDSC modulator therapy so that to boost the subject's immune response before reducing immunosuppressive cell population. A suitable regimen comprises 3 weekly administrations of the immunotherapeutic product composition (J0, J7 and J14) with the MDSC modulator therapy initiated at the very end of immunotherapy or very shortly after over a month period (e.g. from J12/J15 to J42/J45).

In a specific embodiment, the combination of the present invention is used for treating HBV infections, especially a chronic one, relying on administering (a) an immunotherapeutic product comprising an adenovirus encoding HBV antigen(s) and (b) one or more MDSC modulator(s) in an amount sufficient to treat or prevent in a subject in need thereof or alleviate one or more symptoms related to HBV-associated diseases and pathologic conditions, according to the modalities described herein. A preferred combination comprises 3 weekly intradermal, subcutaneous or intramuscular administrations of a composition comprising 10⁹, 10¹⁰ or 10¹¹ vp of an Ad encoding HBc, pol and HBsAg immunogenic domains (especially in the form of a fusion comprising the amino acid of SEQ ID NO: 8) and oral administrations of 1.5 to 100 mg of sildenafil (preferably given at individual doses of 2, 5, 10, 20, 25, 50 or 60 mg per day or every 2 days). More preferably, sildenafil therapy starts at least one week before initiating adenovirus therapy.

The infecting HBV can be from the same genotype, strain or isolate as any HBV from which originates the HBV antigens in use in the present invention (e.g. genotype D) or it can be from a different genotype (e.g. genotype B, C, A or E).

Methods of Treatment

In another aspect, the present invention relates to a composition comprising (a) an immunotherapeutic product composition as described herein for use in combination with (b) one or more MDSC modulator(s) as described herein in an amount sufficient to treat or prevent a disease or a pathologic condition in a subject in need thereof. The present invention also relates to a method of treatment comprising administering a) and b) in combination for treating or preventing a disease or a pathologic condition in a subject in need thereof.

Because of its ability to down regulate MDSC-mediated immunosuppressive signals, thus providing an enhancement of the subject's immune response, the immunostimulatory combination of the invention is/are particularly useful for treating or preventing diseases for which an effective immune system plays a crucial role for reversing the disease state, especially the ones characterized by MDSC-mediated immunosuppression. Therefore, the present invention also provides methods and use for treating a subject having a condition that would benefit from upregulation of an immune response, comprising administering to the subject (a) in combination with (b), such that the condition would benefit from upregulation provided by b) on the immune response elicited by a).

Targeted Diseases or Pathological Conditions

A “disease” (and any form of disease such as “disorder” or “pathological condition”) is typically characterized by identifiable symptoms. Exemplary diseases include, but are not limited to, proliferative diseases, infectious diseases and acute or chronic inflammatory diseases.

As used herein, the term “proliferative disease” encompasses any disease or condition resulting from uncontrolled cell growth and spread including cancers as well as diseases associated to an increased osteoclast activity (e.g. rheumatoid arthritis, osteoporosis, etc) and cardiovascular diseases (restenosis that results from the proliferation of the smooth muscle cells of the blood vessel wall, etc). The term “cancer” may be used interchangeably with any of the terms “tumor”, “malignancy”, “neoplasm”, etc. These terms are meant to include any type of tissue, organ or cell, any stage of malignancy (e.g. from a prelesion to stage IV) encompassing solid and blood borne tumors and primary and metastatic tumors whatever their nature and their degree of anaplasia. Representative examples of cancers that may be treated using the immunostimulatory combination and methods of the invention include, without limitation, carcinoma, lymphoma, blastoma, sarcoma, and leukemia and more particularly bone cancer, gastrointestinal cancer, liver cancer, pancreatic cancer, gastric cancer, colorectal cancer, esophageal cancer, oro-pharyngeal cancer, laryngeal cancer, salivary gland carcinoma, thyroid cancer, lung cancer, cancer of the head or neck, skin cancer, squamous cell cancer, melanoma, uterine cancer, cervical cancer, endometrial carcinoma, vulvar cancer, ovarian cancer, breast cancer, prostate cancer, cancer of the endocrine system, sarcoma of soft tissue, bladder cancer, renal cancer, kidney cancer and cancers of the central and peripheral nervous systems, including astrocytomas, glioblastomas, medulloblastomas and neuroblastomas. The present invention is particularly useful for the treatment of a cancer selected from the group consisting of renal cancer (e.g. clear cell carcinoma), bladder cancer, prostate cancer (e.g. hormone refractory prostate adenocarcinoma), breast cancer (e.g. metastatic breast cancer), colorectal cancer, lung cancer (e.g. non-small cell lung cancer), liver cancer (e.g. hepatocarcinoma), gastric cancer, pancreatic cancer, melanoma, ovarian cancer and glioblastoma, and especially metastatic ones. In certain embodiment, a combination comprising a MUC-1 encoding vector (e.g. TG4010) and a PDE5 inhibitor such as sildenafil is particularly appropriate for the treatment of cancers that overexpress MUC-1 (especially hypoglycosylated form thereof) such as renal, lung and breast cancers.

Treatment of inflammatory diseases such as Alzheimer, arthritis (e.g. rheumatoid arthritis), asthma, atherosclerosis, Crohn disease, irritable bowel syndrome, systemic lupus erythematous, nephritis, Parkinson disease and ulcerative colitis can also be envisaged in the context of the present invention.

Infectious diseases result from an infection with a pathogenic organism (e.g. bacteria, parasite, virus, fungus, etc.). Representative examples of infectious diseases that may be treated using the combination and methods of the invention include, without limitation, a) viral diseases such as those resulting from infection by an orthohepadnavirus (e.g. HBV), a papillomavirus (HPV), a poxvirus causing variola or chicken pox, an enterovirus, a retrovirus such as HIV causing AIDS, a flavivirus (e.g. causing Japanese encephalitis, hepatitis C, dengue and yellow fever), an orthomyxovirus (e.g. influenza virus), a paramyxovirus (e.g. parainfluenzavirus, mumps virus, measles virus and respiratory syncytial virus (RSV)), a coronavirus (e.g. SARS), rhabdovirus and rotavirus; b) diseases resulting from infection by bacteria, for example, Escherichia, Enterobacter, Salmonella, Staphylococcus, Shigella, Listeria, Aerobacter, Helicobacter, Klebsiella, Proteus, Pseudomonas, Streptococcus, Chlamydia, Mycoplasma, Pneumococcus, Neisseria, Clostridium, Bacillus, Corynebacterium, Mycobacterium, Campylobacter, Vibrio, Serratia, Providencia, Chromobacterium, Brucella, Yersinia, Haemophilus, or Bordetella; c) fungal diseases including but not limited to candidiasis, aspergillosis, histoplasmosis, cryptococcal meningitis; and d) parasitic diseases including but not limited to malaria, leishmaniasis, toxoplasmosis, and trypanosome infection. The present invention is particularly useful for treatment of viral infections associated with any of HPV, HCV or HBV virus and of bacterial infection associated with Mycobacterium, and especially Mycobacterium tuberculosis (Mtb)-associated latent or chronic tuberculosis.

In the context of the invention, the combination and methods of the invention provide a therapeutic benefit to the treated subject which can be evidenced by an observable improvement of the clinical status over the baseline status or over the expected status if not treated with the combination described herein. An improvement of the clinical status can be easily assessed by any relevant clinical measurement typically used by physicians or skilled healthcare staff. The appropriate measurements such as blood tests, analysis of biological fluids and biopsies as well as medical imaging techniques can be used to assess a clinical benefit. They can be performed before the administration (baseline) and at various time points during treatment and after cessation of the treatment. Such measurements are evaluated routinely in medical laboratories and hospitals and a large number of kits is available commercially (e.g. immunoassays, quantitative PCR assays).

In the context of the invention, the therapeutic benefit can be transient (for one or a couple of months after cessation of administration) or sustained (for several months or years). As the natural course of clinical status may vary considerably from a subject to another, it is not required that the therapeutic benefit be observed in each subject treated but in a significant number of subjects (e.g. statistically significant differences between two groups can be determined by any statistical test known in the art, such as a Tukey parametric test, the Kruskal-Wallis test the U test according to Mann and Whitney, the Student's t-test, the Wilcoxon test, etc).

For example, when the method aims at treating a cancer, a therapeutic benefit can be correlated with an increase of the survival rate, a reduction in the tumor number; a reduction of the tumor size, a reduction in the number or extent of metastases, an increase in the length of remission, a stabilization (i.e. not worsening) of the state of disease, a delay or slowing of disease progression or severity, a prolonged survival, a better response to the standard treatment, an improvement of quality of life, a reduced mortality, etc., in the group of patients treated with the combination of the present invention or according to the methods of the present invention with respect to those non treated or treated with only one entity of the combination.

When the method aims at treating an infectious disease, a therapeutic benefit can be evidenced by, for instance, a decrease of the amount of the infecting pathogenic organism quantified in blood, plasma, or sera of a treated subject, and/or a stabilized (not worsening) state of the infectious disease (e.g. stabilization of inflammatory status), and/or the reduction of the level of specific serum markers (e.g. decrease of alanine aminotransferase (ALT) and/or aspartate aminotransferase (AST) associated with liver poor condition usually observed in chronic hepatitis B or C), decrease in the level of any antigen associated with the occurrence of an infectious disease and/or the appearance or the modification of the level of antibodies to the pathogenic organism and/or the release of signals by immune cells (e.g. cytokines) and/or an improved response of the treated subject to conventional therapies (e.g. antibiotics, nucleoside analogs, etc.) and/or a survival extension as compared to expected survival if not receiving the combination treatment.

The present invention also relates to a method for decreasing the levels of HBsAg in the serum of a subject diagnosed as having an HBV infection using the combination of the present invention. The levels of HBV seromarker can be evaluated routinely in medical laboratories and hospitals and a large number of kits is available commercially (e.g. immunoassays developed by Abbott Laboratories, Organon Technika). In a specific embodiment, the method of the present invention permits to decrease the serum HBsAg level in a chronically infected patient by at least 0.5 log₁₀ and preferably by at least 0.7 log₁₀ (e.g. at least one log for at least 2 months) as compared to before treatment.

The present invention also relates to a method for decreasing HBV viral load in the serum of a subject diagnosed as having an HBV infection comprising administering the combination of the invention. For general guidance, the HBV viral load can be determined using a quantitative PCR assay or any other methodology accepted in the art (e.g. Roche Ampli Prep/Cobas taqman assay v2.0, Abbott real-time hepatitis B virus performance assay). In a specific embodiment, the method of the present invention permits to decrease the serum HBV DNA level in a chronically infected patient by at least 0.5 log₁₀ and preferably by at least 0.7 log₁₀ (e.g. at least one log for at least 2 months) as compared to before treatment. The method of the present invention may also interfere with the formation of covalently closed circular (ccc) DNA.

The present invention also relates to a method for increasing the levels anti-HBV antibodies (e.g. anti-HBc, anti-HBs antibodies) in the serum of a subject diagnosed as having an HBV infection using the combination of the present invention. The levels of anti-HBc or HBs antibodies can be evaluated routinely in medical laboratories and hospitals and a large number of kits is available commercially (e.g. immunoassays developed by BioMérieux, Abbott, Roche, BioRad, DiaSorin). The administration(s) of the combination of the invention desirably result(s) in at least a transient increase the level of serum antibodies (e.g. of at least 0.5 log₁₀) as compared to the antibody level measured at baseline. It may also provide an increase of the number of responders (subjects showing anti HBc or HBs antibodies) as compared to the control group.

The present invention also relates to a method for increasing multispecific T cell response (against one or more HBV antigens) in a subject diagnosed as having an HBV infection using the combination of the present invention. For general guidance, anti-HBV T cell response can be evaluated routinely, for example by IFN-g ELISpot assays using suitable HBV peptides or pool thereof. A large number of kits is available commercially (e.g. BD Biosciences, R&D Systems, Abcam). The administration(s) of the combination of the invention desirably result(s) in at least a transient increase of anti-HBV T cell response as compared to the response measured at baseline or an increase in the magnitude of the anti-HBV response (subjects showing response to more than one HBV antigen) and/or an increase of the number of responders (e.g. by a factor of at least 1.5) as compared to the control group or the group treated with the immunotherapeutic product alone.

In a further aspect, the present invention provides a method for treating an infectious disease comprising one or more administration of a PDE5 inhibitor in an amount sufficient to treat or prevent the infectious disease in a subject in need thereof. Therefore, the present invention also relates to a PDE-5 inhibitor for use for treating or preventing an infectious disease, especially a chronic infection disease such as a chronic hepatitis B, e.g., by inhibiting a MDSC-mediated immunosuppressive signal.

In one embodiment, such method or use according to the invention results in the down regulation of a MDSC-mediated immunosuppressive signal.

In a preferred embodiment, said PDE5 inhibitor is sildenafil (or analog thereof) as described herein.

In another embodiment, such method or use according to the invention may be performed in combination with an immunotherapeutic product composition such as one described herein.

Method for Inducing an Immune Response

In another aspect, the present invention encompasses a method of inducing or stimulating an immune response comprising administering to a subject a) a composition comprising an immunologically effective amount of an immunotherapeutic product as described herein and (b) one or more MDSC modulator(s) as described herein. Preferably, b) therapy is initiated prior to initiating a).

In one embodiment, the method of the present invention aims at enhancing cytotoxic T cell activity toward a diseased cell and/or down-regulating immunosuppressive activity mediated by MDSCs. Such a method is particularly appropriate for treating cancer or infectious diseases, and especially a chronic HBV infection.

In one embodiment, the induced or stimulated immune response can be specific (i.e. directed to specific antigens carried by the immunotherapeutic product) and/or non-specific (innate), humoral and/or cellular. In the context of the invention, the immune response is preferably a T cell response CD4+ or CD8+-mediated or both and/or a humoral response (production of antibodies).

The ability of the combination and methods described herein to induce or stimulate an immune response can be evaluated either in vitro (e.g. using biological samples collected from the subject) or in vivo using a variety of direct or indirect assays which are standard in the art. For a general description of techniques available to evaluate the onset and activation of an immune response, see for example Coligan et al. (1992 and 1994, Current Protocols in Immunology; ed J Wiley & Sons Inc, National Institute of Health or subsequent editions). Several assays can be used to detect immune responses including, e.g. ELISA (enzyme-linked immunosorbent assay), ELISpot (enzyme-linked immunospot) and ICS (intracellular cytokine staining), multiparameter flow or mass cytometry. The ability to stimulate a humoral response may be determined by antibody binding and/or competition in binding (see for example Harlow, 1989, Antibodies, Cold Spring Harbor Press). One may also evaluate the representability and/or the level of activation of different immune cell populations involved in immune response using various available antibodies against surface markers. Evaluation of cellular immunity can be performed for example by quantification of cytokine(s) produced by activated T cells including those derived from CD4+ and CD8+ T-cells. Cytokine profile analysis can also be performed, e.g. by multiplex technologies or ELISA; proliferative capacity of T cells can be determined by [³H] thymidine incorporation assay or CellTrace staining; cytotoxic capacity for antigen-specific T lymphocytes can be assayed in a sensitized subject or by immunization of appropriate animal models. RNA biomarker analysis can be performed by quantitative real-time or digital PCR.

In a particular embodiment, the combination and methods of the invention may be employed to improve the innate or specific immune response and/or to block or inhibit immunosuppressive environment. Said induction or enhancement of the immune response is preferably correlated with an increase of immune effector cells and/or a change in the cytokine environment.

The down-regulation of the immunosuppressive environment may be evaluated by a decrease of the number of immunosuppressive cells especially at or at close proximity of the injection site or in organs such as liver and spleen after administration of the combination of the invention.

In specific embodiments employing an immunotherapeutic product composition encoding cancer antigen(s) in combination with a PDE5 inhibitor such as sildenafil, the methods and use according to the invention may result in an inhibition of cancer cell growth, and/or a reduction of tumor volume and/or an enhancement of the subject's survival.

In specific embodiments employing an immunotherapeutic product composition encoding HBV antigens (e.g. HBc and pol antigens and HBs immunogenic domains such as the fusion comprising the amino acid shown in SEQ ID NO: 8) in combination with a PDE5 inhibitor such as sildenafil, the methods and use according to the invention may result in an increase in the number of intrahepatic HBV-specific T cells (CD8+ or CD4+ T cells), in particular functional T cells producing IFNγ (e.g. specific for HBV pol and/or core antigens). Preferably, the increase is of at least 1.2, 1.3, 1.4, 1.5, 1.6, 1.7, 1.8, 1.9, 2, 2.1, 2.2, 2.3, 2.4 or 2.5 fold in the subject treated with such a combination as compared to the administration of the immunotherapeutic product composition in the absence of sildenafil.

In any of the methods according to this aspect of the invention, the combination of the present invention can be administered in association with any conventional therapeutic modalities which are available for treating or preventing the targeted disease or pathological condition. Such conventional therapy may be administered to the subject concomitantly, prior to or subsequent to the combination or method according to the invention. Representative examples of conventional therapy include, without limitation, chemotherapy conventionally used for treating cancers, antibiotics, antimetabolites, antimitotics, antivirals, cytokines, chemokines, monoclonal antibodies, cytotoxic agents as well as siRNA and antisense polynucleotides).

According to an advantageous embodiment, especially when the immunotherapeutic product encodes HBV antigens, the combination or methods of the present invention may be used in association with a standard of care. Representative examples of such standard of care treatment include without limitation cytokines (e.g. IFNalpha, pegylated IFNa2a or 2b such as Pegasys (Roche), Pegintron (Schering Plough) or IntronA (Schering Plough)) and nucleos(t)ide analogs (NUCs) such as lamivudine, entecavir, telbivudine, adefovir, adefovir dipivoxil, tenofovir disoproxil fumarate, tenofovir disoproxil aspartate, tenofovir alafenamide fumarate, tenofovir exalidex, pradefovir, besifovir, clevudine or combinations of these, for example Truvada. The treatment with NUCs is only partially effective (infection resolution is observed in only 3-5% of subjects after 1 year of treatment) and needs long term therapy (may be life-long). It is expected that association with the combination of the invention brings an immune dimension that would permit to complement NUC's action on viral replication, thus resulting in an improvement of such treatment (e.g. by decreasing doses of NUCs or length of NUC treatment required to achieve a therapeutic benefit) or an increase of the percentage of infection resolution (e.g., greater than 5%).

The combination and methods of the invention can also be used in association with radiotherapy. Those skilled in the art can readily formulate appropriate radiation therapy protocols and parameters (see for example Perez and Brady, 1992, Principles and Practice of Radiation Oncology, 2nd Ed. JB Lippincott Co; using appropriate adaptations and modifications as will be readily apparent to those skilled in the field). The types of radiation that may be used in cancer treatment are well known in the art and include electron beams, high-energy photons from a linear accelerator or from radioactive sources such as cobalt or cesium, protons, and neutrons.

In another aspect, the present invention also provides a kit of parts comprising a) an immunotherapeutic product composition and b) one or more MDSC modulator(s) together with instructions for use. In one embodiment, a kit includes at least the immunotherapeutic product composition disclosed herein in a first container and the one or more MDSC modulator(s)) described herein in a second container. The first container is preferably sterile glass or plastic vial and the second tablets. A preferred kit comprises an Ad-based immunotherapeutic product (e.g. a Ad5 virus such as TG1050 expressing a fusion of HBc, pol and HBs immunogenic domains) and sildenafil. Optionally, the kit can include suitable devices for performing proper administration of the individual components and/or a package insert including information concerning these components and their dosage.

All of the above cited disclosures of patents, publications and database entries are specifically incorporated herein by reference in their entirety. Other features, objects, and advantages of the invention will be apparent from the description and drawings, and from the claims. Other features and advantages of the invention will become apparent from the following detailed description, taken in conjunction with the accompanying drawings, which illustrate, by way of example, various features of embodiments of the invention.

### EXAMPLES

### 1. Material and Methods of Examples 1 and 2

The constructions described below are carried out according to the general genetic engineered and molecular cloning techniques detailed in Maniatis et al. (1989, Laboratory Manual, Cold Spring Harbor Laboratory Press, Cold Spring Harbor N.Y. or subsequent editions) or according to the manufacturer's recommendations when a commercial kit is used. PCR amplification techniques are known to the person skilled in the art (see for example PCR protocols—A guide to methods and applications, 1990, published by Innis, Gelfand, Sninsky and White, Academic Press).

1.1. Vectors Constructions and Production

TG1050 (or AdTG18201 under its research name) illustrated hereinafter was engineered to express a fusion of a truncated Core polypeptide (aa 1-148) with a mutated polymerase polypeptide (designated Poll comprising two internal deletions (from positions 538 to 544 and from positions 710 to 742) and 4 amino acid substitutions (D689H, V769Y, T776Y and D777H respectively) and with two immunogenic Env domains (Env1 and Env2, respectively extending from amino acids 14 to 51 and from amino acids 165 to 194 of the HBs protein) inserted in place of the deleted pol regions as represented in SEQ ID NO: 8. All originate from HBV strain Y07587 which sequence is described in international databases (Genbank Y07587) and in different publications. It is a genotype D virus of serotype ayw.

More specifically, a synthetic gene encoding a Coret-Pol-Env1-Pol-Env2-Pol fusion protein was synthesized by GENEART (Regensburg, Germany). This fragment was inserted into the NheI and NotI restriction sites of an adenoviral shuttle plasmid (pTG13135) containing a CMV-driven expression cassette surrounded by adenoviral sequences (adenoviral nucleotides 1-454 and nucleotides 3513-5781 respectively) to allow further generation of the vector genome by homologous recombination (Chartier et al., 1996, J. Virol. 70:4805). The resulting plasmid was called pTG18188.

An adenoviral vector was then obtained by homologous recombination between pTG18188 digested by Bst1107I and Pad and pTG15378 (encoding the complete adenoviral genome) linearized by ClaI digestion. This final adenoviral vector is E3 (nucleotides 28593-30464) and E1 (nucleotides 455-3512) deleted, with the E1 region replaced by the expression cassette containing, from 5′ to 3′, the CMV immediate-early enhancer/promoter, a chimeric human β-globin/IgG intron (as found in pCI vector available in Promega), the synthetic gene sequence encoding the Coret-Pol-Env1-Pol-Env2-Pol and the SV40 late polyadenylation signal. The resulting adenoviral vector (AdTG18201) was generated by transfecting the Pad linearized viral genomes into an E1 complementation cell line. Virus propagation, purification and titration was made as described in Erbs et al. (2000, Cancer Res. 60: 3813). AdTG18201 is described in Martin et al. (Gut, 2015, 64(12): 1961-71) and in WO2013/007772).

1.2. Antiviral and Immunological Responses Evaluation in a Mouse Model

1.2.1 HBV-Persistent Mouse Model

The HBV persistent mice used in the study were described by Dion et al. (2013, J Virol, 87(10):5554-63). The model is based on the introduction in mice of an adeno-associated virus (AAV) encoding for a full length HBV genome (AAV2/8-HBV) and causing the production of infectious HBV particles in mouse livers. This allows the analysis of HBV-specific viral parameters (HBsAg, HBeAg, HBcAg and viremia) as well as immunological read-outs (ICS, ELISpot or humoral immune responses).

More specifically, C57BL/6J mice were infected with 5×10¹⁰ vg of AAV2/8-HBV in the retro-orbital venous sinus. Blood samples were taken before treatment (at days 14 and 28 after AAV2/8-HBV infection, sera were sampled to allocate mice per group based on their level of HBsAg at those times) and post treatment for about 1 or 2 months (at days 49, 63 and 76 or at days 50, 64, 78, 92 and 104).

1.2.2. Administration Protocols

1.2.2.1 AdTG18201 Vaccination Protocol

Mice were subcutaneously (sc) immunized with 2×10⁹ vp of AdTG18201 (3 weekly sc administration at days 36, 43 and 50).

1.2.2.2 Sildenafil Administration Protocol

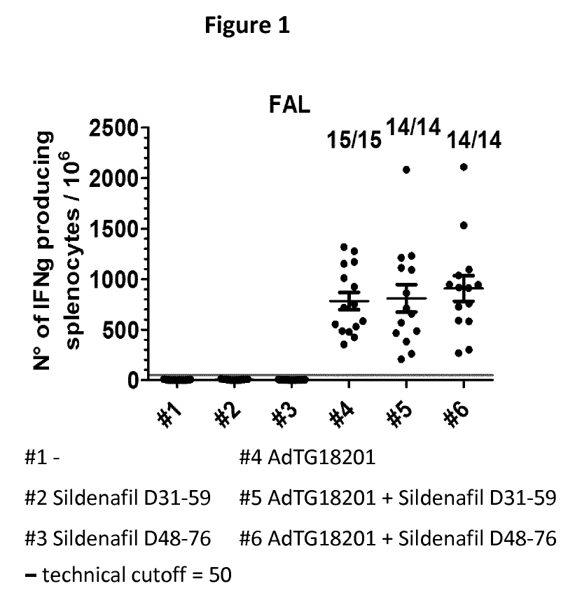

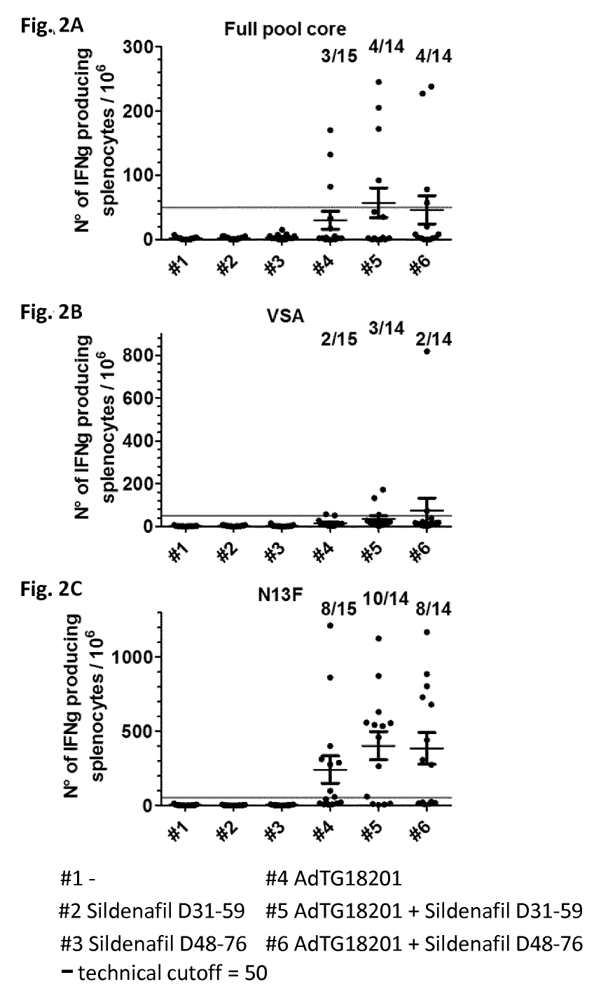

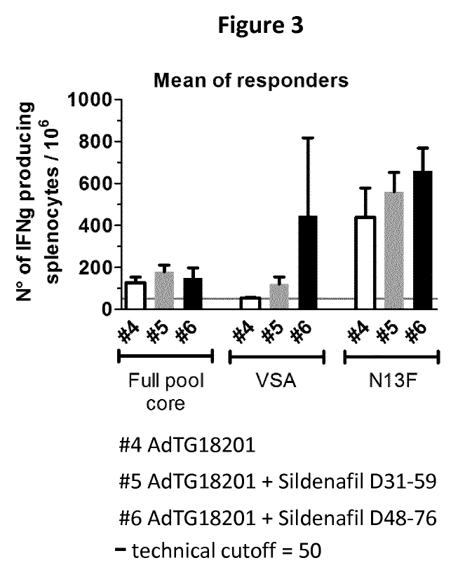

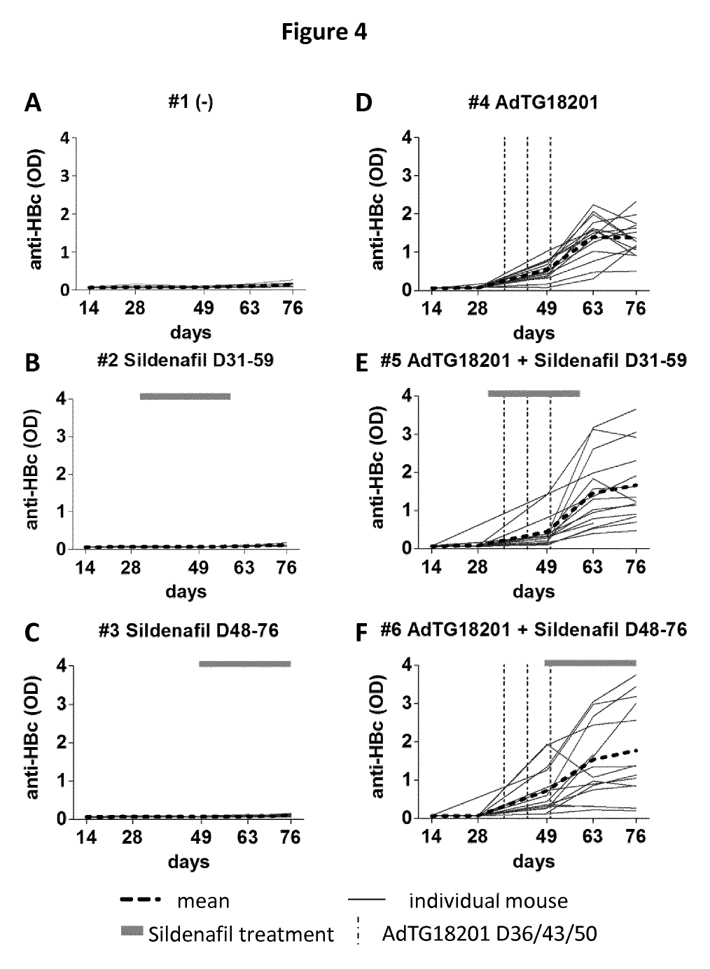

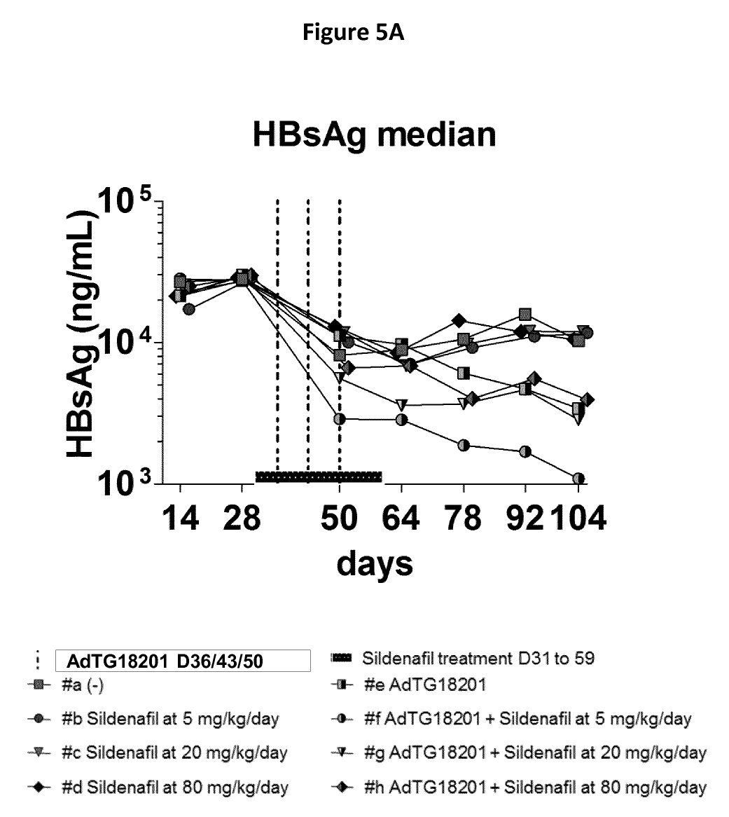

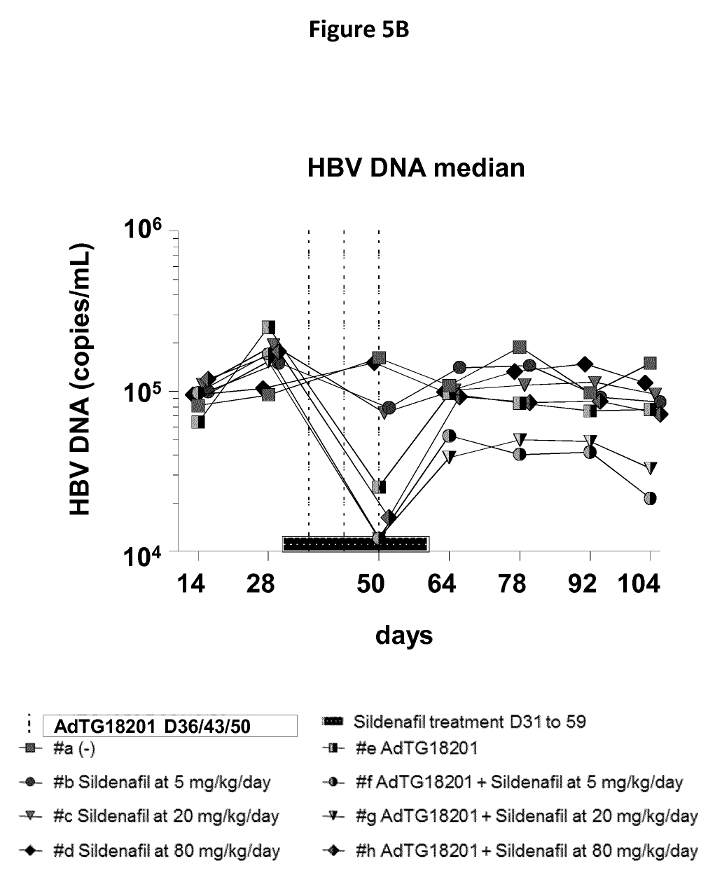

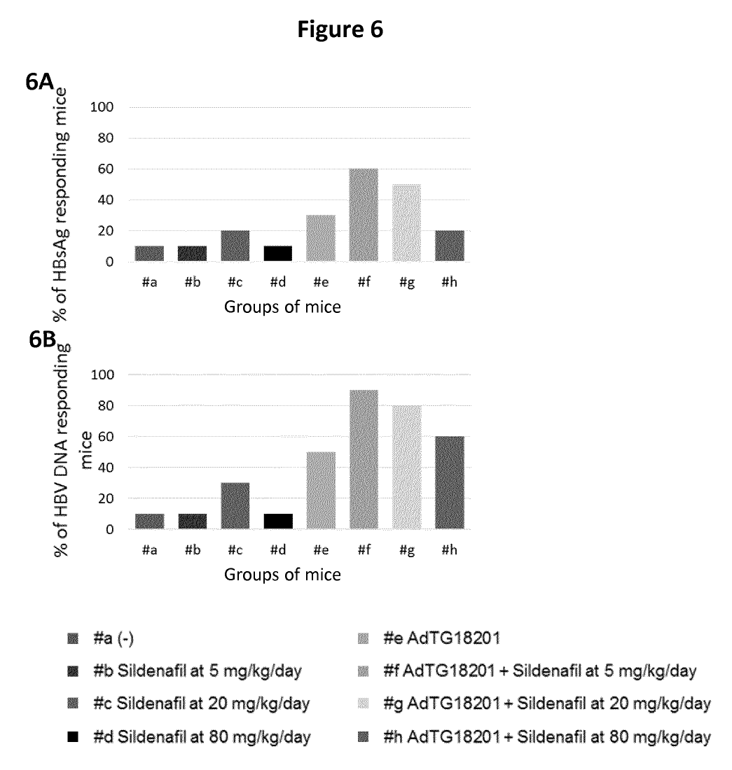

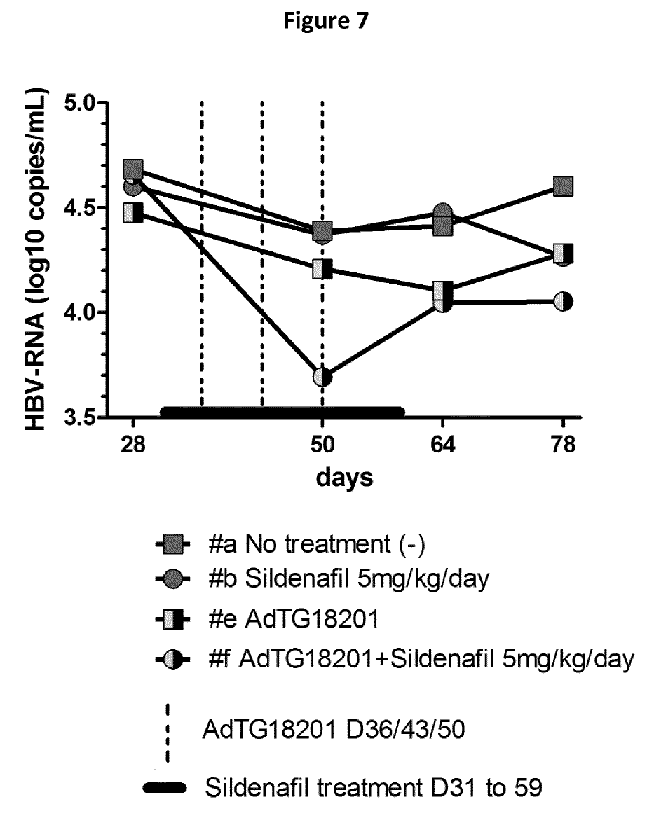

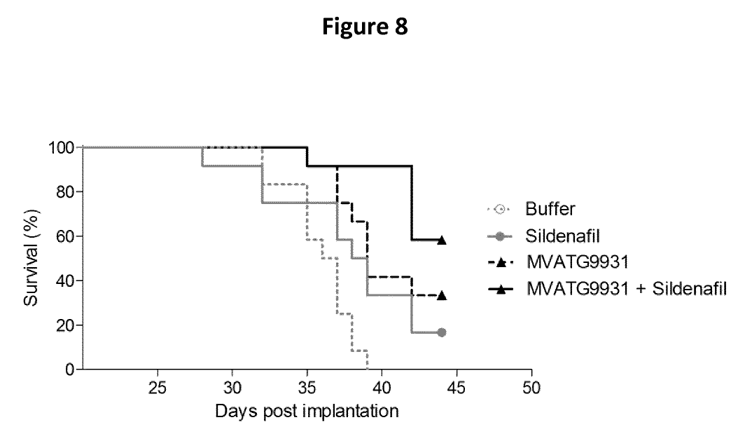

Sildenafil (Sildenafil citrate, Sigma Aldrich) was administered in drinking water from day 31 to 59 or from day 48 to 76 at a concentration of 0.13 mg/mL (corresponding to 20 mg/kg/day for a mouse of 20 g) during the 1^{st} experiment (FIGS. 1 to 4). For the 2^{nd} experiment (FIGS. 5 and 6), Sildenafil citrate, Euromedex) was administered in drinking water from day 31 to day 59 at a concentration of 0.035 mg/mL (corresponding to 5 mg/kg/day for a mouse of 21 g), or a concentration of 0.14 mg/mL (corresponding to 20 mg/kg/day for a mouse of 21 g) or a concentration of 0.56 mg/mL (corresponding to 80 mg/kg/day for a mouse of 21 g). The drinking bottle had been replaced with freshly prepared sildenafil solution twice a week.

1.2.3 Immunological Parameter Monitoring

1.2.3.1 Anti-HBc Antibody Determination

96-well Immulon 4HBX plates (Thermo Scientific) were coated overnight at +4° C. with 100 μL of recombinant HBc (Prospec) diluted to 1 μg/mL in BupH Carbonate-Bicarbonate buffer (Thermo Scientific Cat#28382). Next day, plates were saturated with 200 μL/well of SuperBlock blocking buffer (Thermo Scientific)+0.05% Tween 20 for 2 h at room temperature (RT). Plates were emptied and filled with 80 μL of SuperBlock+0.05% Tween 20. Twenty microliters of tested sera dilution (1/400) in PBS-0.05% Tween were transferred to triplicate wells giving final dilution of 1/2000. Plates were incubated at RT for 2 h and washed 3 times with 200 μL/well of washing buffer (1×TBS diluted from 20× solution (Thermo Scientific)+0.05% Tween 20). One hundred microliters per well of HRP conjugated goat-anti-mouse-IgG (Jackson Immuno Research Cat#115-035-003) diluted (1/10000) in SuperBlock plus 0.05% Tween 20 were added and plates were incubated 1 h at RT. After 6 washings with washing buffer, 100 μL per well of TMB Substrate Solution (Calbiochem) were added and reaction was developed at RT. Ten minutes later, reaction was stopped by adding of 100 μL per well of 1N HCl and optic density was measured at 450 nm with Tecan Infinity 200 reader. Data were presented as the mean of triplicate wells measurements without background subtraction. Wells with saturated signal were assigned OD=3.0 for mean calculation and presentation needs.

Diluted normal mouse serum was used as negative control. Most of plates contained also triplicate wells of “no serum” (PBS) control. Diluted normal mouse serum spiked with 10 μg/mL of anti-HBc monoclonal antibodies (Santa-Cruz clone CL-5) was used as positive control.

1.2.3.2 Peptides Used for ELISpot Assay

Peptides used for cell stimulation ex vivo are either short peptides of 8 to 10 amino acids or long peptides of 15 amino acids included in peptide libraries covering HBV antigens of interest.

Peptides corresponding to described H-2^{b}-restricted epitopes of Pol protein VSA (position 419 to 428, VSAAFYHLPL; SEQ ID NO: 10) and N13F (position 44 to 58, NLNVSIPWTHKVGNF; SEQ ID NO: 11) were synthesized by Eurogentec (Belgium) and were dissolved in 100% DMSO (Sigma) at a concentration of 10 mM.

Peptide library covering the whole Core (from residues 1 to 183) was synthesized by ProImmune (Oxford, United Kingdom). The Core library was composed of 15 mer peptides overlapping by 11 amino acids. Each crude peptide was dissolved in 100% DMSO (Sigma) at a concentration varying from 17 to 48 mg/mL according to the peptide. Altogether, HBc protein was covered by 43 overlapping peptides.

1.2.3.3 IFNg ELISpot Assay

Splenocytes from immunized mice were collected at day 76 following AAV-HBV injection (corresponding to 26 days post AdTG18201 injections) and red blood cells were lysed (Sigma). 2×10⁵ cells per well were cultured in triplicate for 40 h in Multiscreen plates (Millipore, MSHA) coated with an anti-mouse IFNγ monoclonal antibody (BD Biosciences; 10 μg/mL) in MEM culture medium (Gibco) supplemented with 10% FCS (JRH, 12003-100M), 80 U/mL penicillin/80 μg/mL streptomycin (PAN), 2 mM L-glutamine (Gibco), lx non-essential amino acids (Gibco), 10 mM Hepes (Gibco), 1 mM sodium pyruvate (Gibco) and 50 μM β-mercaptoethanol (Gibco) and in presence of 10 units/mL of recombinant murine IL2 (Peprotech), alone as negative control, or with:
10 μM of a selected H-2^{b} restricted peptide present in HBV antigens encoded by plasmids (VSA and N13F for Pol) or an adenovirus specific peptide (FAL) or an irrelevant one;
a pool of peptides (full pool Core) at a final concentration of 3.9 μg/mL per peptide
5 μg/mL of Concanavalin A (Sigma) for positive control.

IFNg-producing T cells were quantified by cytokine-specific ELISpot (enzyme linked immunospot) assay as previously described (Himoudi et al., 2002, J. Virol. 76: 12735). The number of spots (corresponding to the IFNg-producing T cells) in negative control wells were subtracted from the number of spots detected in experimental wells containing HBV peptides. Results are shown as the mean value obtained for triplicate wells. An experimental threshold of positivity for observed responses (or experimental cutoff) was determined by calculating a threshold value which corresponds to the mean value of spots observed with medium alone+2 standard deviations, reported to 10⁶ cells. A technical cutoff linked to the CTL ELISpot reader was also defined as being 50 spots/10⁶ cells (which is the value above which the CV (coefficient of variation) of the reader was systematically less than 20%). The highest value between the technical cutoff and the experimental threshold calculated for each experiment was taken into account to define the cutoff value of each experiment. Statistical analyses of ELISpot responses were conducted by using a Mann-Whitney test. P values equal or inferior to 0.05 were considered as significant.

1.2.4 Viral Parameter Monitoring

1.2.4.1 HBsAg Determination Protocol

HBsAg levels in mouse serum were assessed using a commercial ELISA kit (Monolisa HBsAg Ultra, Bio-Rad, France) according to the manufacturer's protocol, except that a standard curve was established, to render the test quantitative. Each serum was diluted 1/400, 1/2000, 1/10000 and 1/50000 in PBS 1× 0.05% Tween 20 and the HBsAg concentration was calculated in ng/mL by referrence to the standard curve established with 8 known concentrations of recombinant HBsAg (Hytest, subtype adr) giving a range of HBsAg concentrations between 0.2195 ng/mL and 3.75 ng/mL.

1.2.4.2 HBV DNA Determination Protocol

1.2.4.2.1 HBV DNA+RNA Extraction Protocol

HBV DNA/RNA extraction was performed using the MagMax-96 viral RNA isolation kit from Ambion according to manufacturer's protocol. Briefly, 50 μL of serum was added to 20 μL of reconstituted RNA/DNA binding beads. Plate was shaken for 1 minute on a thermomixer (Eppendorf Thermomixer compact) at 600-700 rpm. Then, 130 μL of lysis/binding solution, spiked with qPCR kit internal extraction control DNA, were added before additional shaking for 10 minutes. Beads were captured on a magnetic stand for 3 minutes and after 4 washes the supernatant was discarded. Residual washing solution was removed by drying the beads (shaking of the plate for 3 minutes at 900 rpm). Then, 50 μL of elution buffer was added (shaking of the plate for 3 minutes at 900 rpm). Viral DNA/RNA containing supernatant was removed from beads (captured twice using the magnetic stand), transferred into a 96-well qPCR-plate and stored at −20° C. before analysis.

1.2.4.2.2 HBV DNA QPCR Protocol

HBV DNA copy number was determined in samples using the PrimerDesign™ Genesig Advanced Kit for Hepatitis B (ref. Path-HBV, PrimerDesign) and the 7500 real time system from Applied Biosystems. Data were analyzed using the 7500 System SDS software v2.0.6.

The internal extraction control DNA was spiked into the lysis buffer from the DNA extraction kit (paragraph 1.2.4.2.1) and co-purified with the DNA sample from mice sera and was used as a positive control for the extraction process. Furthermore, this indicated that PCR inhibitors were not present at high concentration.

The Quantitect Multiplex PCR kit (ref. 204545, Qiagen) was used to carry out the qPCR reactions. The reaction volume was 20 μL.

| Component | Volume | |
|---|---|---|
| 2X PCR master mix from Quantitect Multiplex PCR kit | 10 | μL |
| HBV specific primer/probe mix | 1 | μL |
| Internal extraction control primer/probe mix | 1 | μL |
| H₂O | 3 | μL |
| Sample | 5 | μL |

Standard curve dilution series with HBV positive control and reaction mixes were realized according to supplier's procedure resulting in a dynamic range from 10² to 10⁶ copies.

The amplification protocol was:

| | Step | Time | Temperature |
|---|---|---|---|
| | Enzyme | 15 min | 95° C. |
| | activation | | |
| 50 cycles | Denaturation | 10 sec | 95° C. |
| | Data collection | 60 sec | 60° C. |

The limit of quantification (LOQ) was 100 DNA copies per reaction and the limit of detection (LOD) was 10 copies per reaction.

For quantification analysis, Cq threshold for HBV specific primer/probe (FAM) was set at log ΔRn 0.007236 and for internal extraction control primer/probe (VIC) at log ΔRn 0.0026, where Rn is the fluorescence of sample divided by the fluorescence of the reference and ΔRn is Rn minus the baseline. Results are expressed in number of copies of HBV-DNA per mL serum.

1.2.4.2.3 HBV RNA Determination Protocol

Thirty-two μL of DNA/RNA extraction (1.2.4.2.1) was treated with 2 units of Turbo DNAse (Ambion) for 20 min at 37° C. in a heating block. RNA was purified using the Nucleospin RNA clean-up XS kit (Machery-Nagel) according to the specifications of the manufacturer with a final elution step of 10 μL in RNAse free water. Four and a half μL of the elution were added to 4 μl of Superscript IV VILO master mix (Invitrogen) for the reverse transcription reaction and completed up to 20 μL with water. The reaction was carried out in a thermocycler following the suppliers protocol (10 min at 25° C., 10 min at 50° C. and 5 min at 85° C.). Five μL of the reverse transcription solution was used for qPCR as described in 1.2.4.2.2.

### 2. Results of Examples 1 and 2

### Example 1: Immune Responses Provided by the Combination Treatment of Immunotherapy and MDSC Modulator

In the first experiment, HBV carrier mice (having received one injection of AAV2/8-HBV) were divided in 6 groups of 14 or 15 animals which were treated differently. Groups 4, 5 and 6 were immunized with 3 weekly subcutaneous injections of AdTG18201 (at D36, 43 and 50 post AAV-HBV injection). Mice of groups 2, 3, 5 and 6 received Sildenafil in water from D31 to D59 (groups 2 and 5) or from D48 to D76 (groups 3 and 6). The “no treatment” group 1 was injected with AAV2/8-HBV but was not immunized nor received a sildenafil treatment. To summarize, groups are the followings: Control group 1 did not receive any treatment. Group 2 and 3 received sildenafil from D31 to D59 or from D48 to D76, respectively. Mice from group 4 received AdTG18201 only (3× weekly sc injections) whereas groups 5 and 6 received AdTG18201 and sildenafil from D31 to D59 (concomitantly with AdTG18201 treatment) or from D48 to D76 (at the end of AdTG18201 treatment), respectively.

The ability of AdTG18201 to induce adenovirus (Ad)-specific and HBV-multispecific T cells in animals was assessed by IFNg-ELISpot assay at day 76 following AAV2/8-HBV injection (corresponding to about 1-month post AdTG18201 injections).

FIG. 1 illustrates the IFNg-response to the Ad-specific peptide FAL (SEQ ID NO: 12; FALSNAEDL). As expected, mice not injected with AdTG18201 (groups 1-3) were negative (no response above the cutoff) whereas all AdTG18201-immunized mice (groups 4-6) showed an IFNg-response to the Ad-specific peptide FAL with similar mean values when AdTG18201 was administered alone or in combination with sildenafil.

HBV-multispecific IFNg-responses were monitored by IFNg-ELISpot assay at day 76 post AAV2/8-HBV injection following stimulation with a pool of core peptides (full pool core), the CD8 polymerase-specific VSA peptide (SEQ ID NO: 10) and the CD4 polymerase-specific N13F-peptide (SEQ ID NO: 11). As shown in FIGS. 2A, 2B and 2C, groups 1 to 3 did not show any IFNg producing T responses to any HBV-specific stimulation (Full pool core, polymerase-specific peptides VSA or N13F) while most animals of groups 4-6 treated with AdTG18201 (+/−sildenafil) showed an HBV-specific response above the cut off (represented by the horizontal gray line) whatever the time lines of sildenafil treatment with respect to AdTG18201 injections. Altogether, the combination treatment (AdTG18201+sildenafil) induced anti-core and anti-polymerase responses in a higher percentage of mice compared to AdTG18201 treatment alone. More specifically, 1 or 2 more responder animals were obtained when sildenafil is provided together with the 3 injections of AdTG18201. Especially 10 animals showed an anti-pol IFNg response (N13F) when sildenafil is provided concomitantly with AdTG18201 treatment (group 5) compared to 8 responders in AdTG18201-treated animals (group 4). Not only the incidence but also the strength of the response is increased by combination treatment. The mean values of group 5 and 6 are higher than the mean value of group 4 for all 3 HBV-specific stimulations with an average of 2.0 or 2.7 times stronger response against core and pol peptides in the combination treatment groups 5 or 6 than in group 4, respectively.

FIG. 3 illustrates the mean values of only IFNγ-responder mice defined by >50 detected spots per 10⁶ splenocytes. Particularly, for the 2 polymerase stimulations (VSA, N13F), responder mice displayed higher frequencies of IFNγ-producing specific cells in the combination treatment groups 5 and 6, compared to AdTG18201-treated mice in group 4.

The frequency (%) of mice producing IFNg in response to one, two or three stimulations was calculated in animal groups 4-6. It is noticeable that 14.3% of mice in both combination groups 5 and 6 were able to react to all 3 peptide stimulations (Full pool core, polymerase-specific peptides VSA and N13F) versus 6.7% in group 4 (treated with AdTG18201 only). In other words, the percentage of mice responding to three peptide stimulations (multi anti HBV responses) is augmented by a factor of 2.1 in the combi group with respect to AdTG18201-treated group. In addition, the percentage of IFNγ-non-responders is decreased in the combination groups 5 and 6 (respectively 28.6 or 42.9% of mice did not respond to any stimulation), compared to 46.7% in the AdTG18201-treated group 4.

Humoral responses were also monitored by ELISA following AdTG18201 and sildenafil treatments and anti-HBc antibodies were determined in individual mice of the study groups 1-6, 76 days post AAV2/8-HBV injection using anti-core monoclonal antibodies. As shown in FIG. 4, no anti-HBc antibodies were induced in groups 1-3 which is expected since they did not receive the HBV-expressing adenovirus. In AdTG18201-treated group (group 4), anti-HBc antibodies were detected from day 49 (corresponding to the third injection of AdTG18201) with a maximum peak at day 63. The combination treatment (in groups 5 and 6) induced anti-HBc antibodies with similar kinetics as in group 4 but higher levels were detected in a number of mice. More specifically, 3 (21%) and 5 mice (36%) out of 14 showed very high levels (exceeding the optical density of 2.5) in groups 5 and 6, respectively, which was not observed in AdTG18201-treated group 4. Most of mice in group 5 (86%) and 6 (82%) tend to have increasing levels of anti-HBc antibodies at the end of the experiment (D63 to D76), whereas approximately half of those of group 4 (AdTG18201, 47%) display a decreasing trend.

### Example 2: Antiviral Responses Provided by the Combination Treatment of Immunotherapy and MDSC Modulator

A second set of experiments was carried out with varying doses of sildenafil. More specifically, HBV carrier mice (injected once with AAV2/8-HBV) were divided in 8 groups of 10 animals which were treated differently. Groups e, f, g and h were immunized with 3 weekly subcutaneous injections of AdTG18201 (at D36, 43 and 50 post AAV-HBV injection). Mice of groups b, c, d, f, g and h received Sildenafil in water from D31 to D59 (at 5 mg/kg/day in groups b and f; at 20 mg/kg/day in groups c and g; at 80 mg/kg/day in groups d and h). Group a was injected with AAV2/8-HBV but was not Ad immunized nor received sildenafil (“no treatment” group). To summarize, groups are the followings: Group a did not receive any treatment. Group b, c and d received sildenafil from D31 to D59 at 5, 20 or 80 mg/kg/day, respectively. Mice from group e received AdTG18201 only (3× weekly sc injections) whereas groups f, g and h received AdTG18201 and sildenafil from D31 to D59 (concomitantly with AdTG18201 treatment) at 5, 20 or 80 mg/kg/day, respectively.

Antiviral responses were followed up to D104 post AAV-HBV injection by evaluating the levels of HBsAg and viral load in the sera obtained from these animals. HBsAg was assessed by ELISA and the amount of HBV DNA by qPCR.

FIG. 5A shows HBsAg median values of all groups (a to h) included in the experiment. As expected, no significant HBsAg decrease was observed in control group a (untreated) nor in groups b to d treated with sildenafil only (5, 20 or 80 mg/kg/day). Compared to the control group a, AdTG18201 treatment (group e) provided a regular decrease of the HBsAg level starting after the end of treatment at day 78 to the end of the experimentation (day 104). HBsAg level observed in mice treated with AdTG18201 and 80 mg/kg/day of sildenafil parallels approximately that measured in AdTG18201-immunized animals. The combination treatment of AdTG18201 with 20 mg/kg/day sildenafil (group g) induced a reduction of the HBsAg level which started earlier than with AdTG18201 alone (group e) and stabilized at approximately the same level as the one observed for AdTG18201 alone (group e). The combination treatment in group f (AdTG18201 with the sildenafil dose of 5 mg/kg/day) induced a strong decrease of the HBsAg level, HBsAg median value reaching 1 μg/mL at the end of the experiment (corresponding to more than 1 log decrease between day 28 and day 104).

FIG. 5B shows HBV DNA median values of all groups (a to h) included in the experiment. The same tendency as for HBsAg was observed with HBV DNA levels. More specifically, the median level of viral DNA stabilized around 10⁵ copies/mL serum over the experiment time period in control group a (untreated) and in groups b to d treated with sildenafil only (5, 20 or 80 mg/kg/day). In groups e and h (respectively treated by AdTG18201 alone or associated with 80 mg/kg/day of sildenafil), a strong and transitory decrease of the HBV DNA level was observed at day 50, compared to the control group a (untreated) and sildenafil-treated groups. But the level rose again and rapidly to the same level as before treatment. In contrast, animals of groups f and g treated with AdTG18201 and the 2 lowest tested sildenafil doses (5 and 20 mg/kg/day, respectively) displayed a stronger decrease of viral DNA at day 50 (over 1 log decrease at day 50 as compared to day 28) and this decrease persisted up to 45 days after treatment. Thus, contrary to sildenafil-treated groups b, c and d and AdTG18201-treated group e, the combi treatment provided a significant and sustained reduction of serum viral load (approximately 1 log decrease for group f and between 0.5 and 1 log decrease for group g between day 28 and day 104) especially at doses of sildenafil of 5-20 mg/kg/day.

FIG. 6 illustrates frequencies of HBsAg (FIG. 6A) and HBV DNA (FIG. 6B) responding mice in the different groups of animals, “responding” meaning displaying at least a 0.5 log decrease in HBV DNA or at least a 1 log decrease in HBsAg levels, for, at least, two time points. Concerning HBsAg responses (FIG. 6 A), 6 out of 10 mice (60%) in the combination treatment group f (AdTG18201+5 mg/kg/day Sildenafil) and 5 out of 10 mice (50%) in the combination treatment group g (AdTG18201+20 mg/kg/day Sildenafil) were identified as responding mice whereas only whereas 20% of mice treated by AdTG18201 and sildenafil at 80 mg/kg/day (group h) showed noticeable antiviral responses. By comparison, AdTG18201-treated group (group e) displayed 30% of responding mice.

Concerning HBV DNA responses (FIG. 6 B), 90%, 80% and 60% of mice in the combi groups f-h (treated with AdTG18201 and Sildenafil at doses of 5, 20 and 80 mg/kg/day, respectively) were considered as responding. The percentage of responding mice decreased to 50% in group e treated by AdTG18201 alone.

HBV RNA was measured in six representative mice in selected groups (a, b, e and f) at selected time points. No HBV RNA levels of other mice, other time points or other groups were measured. FIG. 7 shows HBV RNA median values with the same tendency as for HBsAg and HBV DNA (FIG. 5). More specifically, the median level of viral RNA stabilized around 4.5 log 10 copies/mL serum over the experiment in control group a (untreated) and in group b treated with sildenafil only (5 mg/kg/day). In group e (treated with AdTG18201 alone), a decrease of the HBV RNA level was observed at day 64 (4.1 log 10 copies/mL serum), compared to the control group a (untreated) and sildenafil-treated group b. Animals of group f treated with AdTG18201 and the lowest tested sildenafil dose (5 mg/kg/day) induced a stronger decrease of viral RNA at day 50 (3.7 log 10 copies/mL serum, representing a 1-log decrease at day 50 as compared to day 28). Median HBV RNA levels in this group rose moderately at day 64, but stayed below all other groups at day 64 and day 78. Thus, contrary to the sildenafil-treated group b and the AdTG18201-treated group e, the combination treatment provided a sustained reduction of serum viral load.

### 3. Conclusions of Examples 1 and 2

AdTG18201 induced HBV-multispecific IFNγ-producing T cells in an HBV-persistent mouse model. The combination treatment (AdTG18201+sildenafil) induced stronger IFNγ ELISpot responses (2-2.7 times) and led to more IFNγ-responder mice than in the AdTG18201 treatment group. Furthermore, the combination treatment led to higher maximum levels of AdTG18201-induced anti-HBc antibodies (>OD of 2.5 in 21-36% versus 0% in the AdTG18201 treatment group). It should be noted that combi treatment of sildenafil and AdTG18201 did not provide any improvement of the IFNg response against the adenovirus vector with respect to separate treatment (sildenafil or AdTG18201). Moreover, combi treatment of AdTG18201 immunotherapy with 5-20 mg/kg/day of sildenafil provided effective antiviral responses as evidenced by a strong and sustained serum HBsAg and HBV DNA and RNA decrease compared to individual treatment with AdTG18201 or sildenafil and a higher frequency (about 2 fold) of responding mice in the combi groups f and g.

### Example 3: Anticancer Response Provided by the Combination Treatment of Immunotherapy and MDSC Modulator

The therapeutic effect provided by the combination of an MVA immunotherapeutic candidate (MVATG9931) with sildenafil was also tested in a CT26 colon cancer animal model.

MVATG9931 was described in Claudepierre et al. (2014, J. Virol. 88(10): 5242-55). Animals were used between 6 and 10 weeks age. BALB/c mice were IV injected (caudal vein) with 2×10⁵ CT26-MUC1 cells. On day 2 and 9 after tumor challenge, mice were treated with 5×10⁷ pfu of MVATG9931 or Buffer. Sildenafil (Sildenafil citrate, Euromedex) was administered in drinking water from day 0 to 28 at a concentration of 0.52 mg/mL (corresponding to 80 mg/kg/day for a mouse of 20 g). The drinking bottle had been replaced with freshly prepared sildenafil solution twice a week. Animal survival was monitored for more than 45 days. For this purpose, mice were weighed twice per week and sacrificed when reaching 10% weight loss. The experiment was carried out with 12 mice per group. Overall survival rates represented as Kaplan-Meier curves. Animal experiments were conducted in compliance with EU directive 2010/63/EU.

As illustrated in FIG. 8, the survival of mice included in the combo treatment group was increased as compared to the monotherapy groups and the negative control group. More specifically, none of the mice treated with the buffer survive (100% were died before 40 days post tumor implantation). An increase in survival was observed in animals treated with either sildenafil or MVATG9931 with 20% of mice treated with sildenafil and 38% of mice treated with MVATG9931 still alive 44 days post tumor implantation. In contrast, survival of mice treated with both MVATG9931 and sildenafil reached 60% at the same period of time (44 days post tumor implantation).

### BIBLIOGRAPHIC REFERENCES

Adra et al., 1987, Gene 60: 65-74;
Annels et al., 2014, Cancer Immunol Immunother 63(2): 175-83;
Anthony et al., 2011, Vaccine 29: 3558-63;
Antoine et al., 1998, Virol. 244: 365-96;
Bartenschlager et al., 1990, J. Virol. 64: 5324-32;
Bioorg. Med. Chem. Lett. 2000, 10, 1983-1986;
Boukhebza et al., 2012, Vaccines & Immunotherapeutics 8(12): 1746-57;
Brandler and Tangy, 2008, CIMID 31: 271;
Brough et al., 1997, J. Virol., 71: 9206-13;
Bukreyev and Collins, 2008, Curr Opin Mol Ther 10: 46-55;
Carrey et al., 2011, PLoS ONE, 6(7) e22442;
Carrey et al., 2014, Sci Rep 4: 6154 doi 10.1038;
Chakrabarti et al. (1997, Biotechniques 23: 1094-7;
Chartier et al., 1996, J. Virol. 70: 4805-10;
Chen et al., 1997, Proc. Natl. Acad. Sci. USA 94: 1914-8
Chen et al., 2000, Cancer Res. 60: 1035;
Chen et al., 2011, Clin Exp Immunol 166: 134-142;
Cheng et al., 2001, J. Clin. Invest. 108: 669;
Chroboczek et al. (1992, Virol. 186: 280-5;
Claudepierre et al. (2014, J. Virol. 88(10): 5242-55;
Coligan et al. (1992 and 1994, Current Protocols in Immunology; ed J Wiley & Sons Inc, National Institute of Health or subsequent editions);
Corbin et al., 2000, Eur J Biochem 267(9): 2760-7;
Csatary et al., 1999, Anti Cancer Res 19: 635-8;
Damuzzo et al., 2015, Cytometry Part B (Clinical Cytometry) 88B:77-91;
Demotz et al., 1993, Eur. J. Immunol. 23: 425;
Depla et al., 2008, J. Virol. 82: 435;
Desombere et al., 2000, Clin. Exp. Immunol 122: 390;
Dion et al. (2013, J Virol, 87(10):5554-63;
Dreno et al., 2014, PLoS One 9(2): e83670;
Drummer et al., 2008, Mol Ther 16(5): 985-94;
Dudareva et al, 2009, Vaccine 27: 3501-4;
EP 2374460;
EP 2575765;
EP 463 756
EP 812 845;
EP1016711;
EP1418942;
EP2968130;
Erbs et al. 2000, Cancer Res. 60: 3813;
Erbs et al., 2008, Cancer Gene Ther. 15(1): 18-28;
Evans et al., 2004, J Pharm Sci. 93:2458-75,
Facciabene et al., 2012, Cancer Res; 72(9): 2162-71;
Fallaux et al., 1998, Human Gene Ther. 9: 1909-1917;
Fang et al., 2015, J Immunol 195: 4873-83;
Fournillier et al., 2007, Vaccine 25(42): 7339-53;
Gabrilovich and Nagarej, 2009, Nat Rev Immunol 9(3): 162-74;
Ganem and Schneider in Hepadnaviridae (2001) “The viruses and their replication”, pp 2923-2969,
Gao, et al, 2007, J. Chromatogr. Sci., 45:540-543;
Goebel et al., 1990, Virol. 179: 247;
Graham et al., 1997, J. Gen. Virol. 36: 59-72;
Griffin et al., 2001, Field's in Virology, 1401-1441;
Hammond et al, 1997, J. Virol Methods 66: 135-8;
Harlow, 1989, Antibodies, Cold Spring Harbor Press;
Harrop and Carroll, 2006, Front Biosci., 11, 804-817;
Hemminki et al., 2015, Oncotarget 6(6): 4467-81;
Hollstein et al., 1994, Nucleic Acids Res. 22: 3551-5
Hung et al., 2001 Cancer Res. 61: 3698;
Inchauspé et al., 2009, Int Rev Immunol 28(1): 7-19;
Israeli et al., 1993, Cancer Res. 53: 227-30
Jerome et al., 1993, J. Immunol. 151: 1654-62;
Johnson et al., 1993, Virol. 196: 381;
Katoh and Watanabe, 2015, Mediators of Inflammation Article ID 159269;
Katoh et al., 2013, Cancer Cell 24(5): 631-44;
Kaufman et al., 1987, EMBO J. 6: 187-95;
Kawakami et al., 1992, Proc. Natl. Acad. Sci. USA 91: 6458-62
Knipe D M et al, eds. Fields Virology, 4th ed. Philadelphia, Lippincott Williams & Wilkins or subsequent edition;
Kondo and Shimosegawa. 2015, Int J Mol Sci 16: 3307-22;
Kritsch et al., 2005, J. Chromatogr Anal. Technol. Biomed. Life Sci. 822: 263-70;
Kumar and Boyle, 1990, Virology 179: 151-8;
Kwon et al., 1987, Proc. Natl. Acad. Sci. USA 84: 7473-7
Lamb et al., 1982, Nature 300: 66;
Lathers et al., 2004, Cancer Immunol Immunother 53(5): 422-30;
Lee et al., 1997, Biochem. Bioph. Res. Commun. 233(2):401;
Limacher and Quoix (2012, Oncolmmunology 1(5): 791-2);
Lindau et al., 2013, Immunol. 138(2): 105-15;
Loirat et al., 2000, J. Immunol. 165: 4748;
Lusky et al, 1998, J. Virol 72: 2022;
Mamum-Al Mahtab et al., 2008, Hepatobiliary Pancrease Dis Int 5: 457-64;
Maniatis et al. 1989, Laboratory Manual, Cold Spring Harbor Laboratory Press, Cold Spring Harbor N.Y. or subsequent editions;
Martin et al. Gut, 2015, 64(12): 1961-71;
Mayr et al., 1975, Infection 3: 6-14;
Meyer et al., 1991, J. Gen. Virol. 72: 1031-8;
Meyer et al., 2011, Proc Natl Acad Sci 108(41): 17111-6;
Mirza et al., 2006, Cancer Res 66(18): 9299-9307;
Needleman et Wunsch. J. Mol. Biol. 48, 443-453, 1970;
Nefedova et al., 2007 Cancer Res 67(22): 11021-8;
Pallett et al., 2015, Nature Medicine 21(6): 591-600;
Pan et al., 2008, Blood 111(1): 219-28;
PCR protocols—A guide to methods and applications, 1990, published by Innis, Gelfand,
Sninsky and White, Academic Press
Perez and Brady, 1992, Principles and Practice of Radiation Oncology, 2nd Ed. JB Lippincott Co;
Peruzzi et al., 2009, Vaccine 27: 1293-300;
Quoix et al., 2011, The Lancet Oncology 12(12): 1125-33;
Radziwill et al., 1990, J. Virol. 64: 613-20;
Ribi et al., 1986, Immunology and Immunopharmacology of Bacterial Endotoxins, Plenum Publ. Corp., NY, p 407-419;
Rodriguez et al., 1997, J. Virol. 71: 8497;
Rothbard et al., 1989, Int. Immunol. 1: 479;
Schaeffer, 2007, World Gastroenterol. 7: 14;
Schirmbeck et al., 2002, J. Immunol 168: 6253;
Schwarz A, 1962, Am J Dis Child, 103: 216;
Seed, 1987, Nature 329: 840;
Serafini et al., 2006, J Exp med 203(12): 2691-702;
Shi et al., 2005, J Pharm Sci. 94:1538-51,
Sidney et al., 1994, Immunity 1: 751;
Smorlesi, 2005, Gene Ther. 12: 1324;
Stoll-Becker et al, 1997, J. Virol. 71: 5399;
Suader, 2000, J. Am Acad Dermatol. 43:S6;
Sumino et al., 1998, J. Virol. 72: 4931; WO98/56415;
Sutter and Moss, 1992, Proc. Natl. Acad. Sci. USA 89: 10847-51;
Tinsley et al., 2010, Cancer Prevention Res 3(10): 1303-13;
Torresi et al., 2011, J. Hepatol. 54(6): 1273-85;
Ugel et al., 2009, Curr Opin Pharmacol 9(4): 470-81;
US2007/0161085;
U.S. Pat. No. 5,168,062;
U.S. Pat. No. 5,250,534,
U.S. Pat. No. 5,494,807;
U.S. Pat. No. 5,648,226
U.S. Pat. No. 5,747,282
U.S. Pat. No. 5,750,395
U.S. Pat. No. 5,861,381;
U.S. Pat. No. 5,972,597;
U.S. Pat. No. 6,054,438;
U.S. Pat. No. 6,204,383;
U.S. Pat. No. 6,440,422;
U.S. Pat. No. 6,469,012;
U.S. Pat. No. 6,998,252;
U.S. Pat. No. 7,456,009;
U.S. Pat. No. 7,465,454;
U.S. Pat. No. 7,563,447;
U.S. Pat. No. 7,914,979,
Van der Maaden et al., 2012, J. Control release 161: 645-55;
Vordermeier et al., 1992, Eur. J. Immunol. 22: 2631;
WO92/07000;
WO93/03764;
WO94/19011;
WO94/28152;
WO94/28902;
WO95/34671;
WO96/17070;
WO96/27677,
WO97/00326;
WO97/02355;
WO98/00524,
WO98/04727;
WO98/10088;
WO98/22588,
WO98/26048,
WO98/37095;
WO99/03885;
WO99/54481;
WO99/66933;
WO00/40702,
WO00/50573;
WO00/54777;
WO01/019827;
WO03/008533;
WO03/046124;
WO03/053463;
WO2000/075597;
WO2004/111082;
WO2005/001103;
WO2005/007840;
WO2005/042728;
WO2005/056051;
WO2005/067936;
WO2005/071093;
WO2006/0850082;
WO2007/056847;
WO2007/077256,
WO2007/147528;
WO2007/147529;
WO2008/020656;
WO2008/074512;
WO2008/114021;
WO2008/129058;
WO2008/138533,
WO2009/004016,
WO2009/065546;
WO2009/065547;
WO2009/073103;
WO2009/073104;
WO2009/100521,
WO2009/105084;
WO2010/086189;
WO2010/130753;
WO2010/130756;
WO2011/015656;
WO2012/001075;
WO2012/019127;
WO2013/007772;
WO2013/022764,
WO2013/052799;
WO2013/052811;
WO2013/052832;
WO2014/009438;
WO2014/029702;
WO2014/053571;
WO2015/104380;
Xue et al., 1997, The Prostate 30: 73-8
Zhang et al., 2009, J Med Virol. 81 (8): 1477;

1.-25. (canceled)

26. A combination comprising at least (a) a composition comprising a therapeutically effective amount of an immunotherapeutic product and at least (b) one or more MDSC modulator(s).

27. The combination of claim 26, wherein said immunotherapeutic product comprises a plasmid or a viral vector.

28. The combination of claim 26, wherein said viral vector is obtained from a poxvirus or an adenovirus.

29. The combination of claim 28, wherein said immunotherapeutic product comprises a replication-defective adenovirus obtained from a human adenovirus of serotype 5 (Ad5) which is defective for E1 and/or E3 function(s).

30. The combination of claim 26, wherein the immunotherapeutic product includes or encodes one or more antigen(s) selected from the group consisting of cancer antigen(s) and antigen(s) originating from an infectious organism or associated with a disease or condition caused by an infectious organism.

31. The combination of claim 30, wherein said one or more antigens are selected from the group consisting of mucin antigens, human papillomavirus (HPV) antigens, hepatitis C virus (HCV) antigens, hepatitis B virus (HBV) antigens, Mycobacterium tuberculosis (Mtb) antigens; and any combination thereof.

32. The combination of claim 31, wherein said one or more antigens are HBV antigens selected from the group consisting of HBV polymerase, HBc, and HBs antigens.

33. The combination of claim 32, wherein:
a) said HBc antigen comprises an amino acid sequence that is at least 80% identical to SEQ ID NO:1 or SEQ ID NO:2;
b) said polymerase antigen is defective for the polymerase enzymatic activity and/or for the RNaseH activity exhibited by the native counterpart and comprises an amino acid sequence that is at least 80% identical to SEQ ID NO:3, SEQ ID NO:4 or SEQ ID NO:5; and/or
c) said HBsAg antigen consists of one or more HBs immunogenic domain(s) comprising an amino acid sequence that is at least 80% identical to SEQ ID NO:6 or SEQ ID NO:7.

34. The combination of claim 32, wherein said immunotherapeutic product encodes a fusion protein of HBc, pol, and HbsAg.

35. The combination of claim 34, wherein said fusion protein of HBc, pol, and HbsAg comprises an amino acid sequence that is at least 80% identical to SEQ ID NO:8.

36. The combination of claim 35, wherein said immunotherapeutic product is a replication-defective adenovirus comprising inserted in place of the E1 region a nucleic acid molecule placed under the control of a promoter, and encoding a fusion protein comprising an amino acid sequence as shown in SEQ ID NO:8.

37. The combination of claim 26, wherein said one or more MDSC modulator(s) comprises a PDE-5 inhibitor capable of antagonizing the activity of a phosphodiesterase subtype 5 (PDE-5).

38. The combination of claim 37, wherein said PDE-5 inhibitor is selected from the group consisting of avanafil, lodenafil, mirodenafil, sildenafil, actetildenafil, hydroxyacetildenafil, dimethylsildenafil, thiomethisosildenafil, tadalafil, vardenafil, udenafil, zaprinast, icariin, sulfoaildenafil, and benzamidenafil.

39. The combination of claim 26, comprising:
a) a composition comprising from about 10⁷ vp to about 10¹² vp of a virus-based immunotherapeutic product, and
b) from about 1 mg to about 200 mg of a PDE5 inhibitor.

40. The combination of claim 39, comprising:
a) about 10⁹ vp, about 10¹⁰ vp or about 10¹¹ vp of an adenoviral vector encoding one or more HBV antigen(s), and
b) daily or every 2 days doses from about 1.5 mg to about 100 mg of sildenafil, taken in one or more doses of 2 mg, 5 mg, 10 mg, 20 mg, 25 mg, 50 mg, or 100 mg.

41. A method for treating or preventing a disease or a pathologic condition in a subject in need thereof or for treating a subject having a condition that would benefit from upregulation of the immune response, comprising administering to said subject a therapeutically effective amount of a combination comprising at least (a) a composition comprising a therapeutically effective amount of an immunotherapeutic product and at least (b) one or more MDSC modulator(s).

42. The method of claim 41, wherein said immunotherapeutic product is formulated for intradermal, transcutaneous, intramuscular, subcutaneous, or intratumoral administration and wherein said one or more MDSC modulator(s) is formulated for oral, sublingual, or intravenous administration.

43. The method of claim 41, wherein said disease or pathologic condition is selected from the group consisting of proliferative diseases, infectious diseases, and acute or chronic inflammatory diseases.

44. The method of claim 43, wherein:
a) said proliferative disease is a cancer selected from the group consisting of renal cancer, bladder cancer, prostate cancer, breast cancer, colorectal cancer, lung cancer, liver cancer, gastric cancer, pancreatic cancer, melanoma, ovarian cancer, and glioblastoma; or
b) said infectious disease is selected from a viral infection with HPV, HCV, or HBV virus or a bacterial infection with Mycobacterium.

45. The method of claim 41, wherein the MDSC modulator(s) is/are given to the subject before initiating administrations of the immunotherapeutic product.

46. The method of claim 45, wherein the MDSC modulator(s) therapy starts prior to immunotherapeutic product therapy, with continuation of MDSC modulator therapy during immunotherapeutic product therapy.

47. The method of claim 45, wherein the one or more MDSC modulator(s) is given to the subject at least one week before initiating administration(s) of the immunotherapeutic product.

48. The method of claim 41, wherein the administration(s) of the immunotherapeutic product composition is initiated before starting the MDSC modulator(s) therapy.

49. The method of claim 48, wherein the administrations of the one or more MDSC modulator(s) are initiated at the very end of the immunotherapeutic product administration(s) or very shortly after.

50. The method of claim 41, wherein said method comprises a) 3 weekly intradermal, subcutaneous or intramuscular administrations of about 10⁹ vp, about 10¹⁰ vp or about 10¹¹ vp of an adenovirus-based immunotherapeutic product and b) oral administrations of 1.5 to 100 mg of said MDSC modulator(s); wherein said MDSC modulator(s) is sildenafil.

51. The method of claim 41, wherein said combination is administered in association with a conventional therapeutic modality available for treating or preventing the targeted disease or pathological condition.

52. The method of claim 51, wherein the immunotherapeutic product encodes HBV antigens and the combination is administered in association with nucleos(t)ide analogs (NUCs).

53. A method for decreasing the levels of HBsAg and/or HBV viral load in the serum of a subject diagnosed as having an HBV infection, comprising administering to said subject a therapeutically effective amount of a combination comprising at least (a) a composition comprising a therapeutically effective amount of an immunotherapeutic product and at least (b) one or more MDSC modulator(s).

54. The method of claim 53, wherein the immunotherapeutic product is a replication-defective adenovirus comprising inserted in place of the E1 region a nucleic acid molecule placed under the control of a promoter, and encoding a fusion protein of HBc, pol, and HbsAg, and the MDSC modulator(s) comprise(s) a PDE-5 inhibitor.

55. A method for treating or preventing a chronic infectious disease in a subject in need thereof or for reversing immunosuppression in a subject having a chronic infectious disease, comprising administering to said subject a PDE-5 inhibitor.

56. The method of claim 55, wherein said infectious disease is chronic hepatitis B.

57. The method of claim 55, wherein the PDE-5 inhibitor is administered in association with a conventional therapeutic modality available for treating or preventing said chronic infectious disease.
